(19) United States
(12) Patent Application Publication
(10) Pub. No.: US 2010/0169025 A1
(43) Pub. Date: Jul. 1, 2010

(54) METHODS AND GENE EXPRESSION SIGNATURE FOR WNT/B-CATENIN SIGNALING PATHWAY

(76) Inventors: William T. Arthur, Perkasie, PA (US); Brian Roberts, Brookline, MA (US); Rachel Needham, Seattle, WA (US); Hongyue Dai, Chestnut Hill, MA (US)

Correspondence Address: MERCK, P O BOX 2000, RAHWAY, NJ, 07065-0907, US

(21) Appl. No.: 12/641,486

(22) Filed: Dec. 18, 2009

Related U.S. Application Data
(63) Continuation-in-part of application No. 12/586,208, filed on Sep. 17, 2009.
(60) Provisional application No. 61/195,811, filed on Oct. 10, 2008.

(51) Int. Cl. G06F 19/00; C12Q 1/68; G06N 5/02

(52) U.S. Cl. 702/19; 435/6; 706/54

(57) ABSTRACT

Methods, biomarkers, and expression signatures are disclosed for assessing the regulation status of Wnt/β-catenin signaling pathway in a cell sample or subject. More specifically, several aspects of the invention provide a set of genes which can be used as biomarkers and gene signatures for evaluating Wnt/β-catenin pathway deregulation status in a sample; classifying a cell sample as having a deregulated or regulated Wnt/β-catenin signaling pathway; determining whether an agent modulates the Wnt/β-catenin signaling pathway in sample; predicting response of a subject to an agent that modulates the Wnt/β-catenin signaling pathway; assigning treatment to a subject; and evaluating the pharmacodynamic effects of therapies designed to regulate Wnt/β-catenin pathway signaling.

This is a continuation-in-part of U.S. patent application Ser. No. 12/586,208, filed on Sep. 17, 2009, which in turn claims benefit of U.S. Provisional Patent Application Ser. No. 61/195,811 filed on Oct. 10, 2008, each of which is incorporated by reference herein in its entirety.

The sequence listing of the present application is submitted electronically via EFS-Web as an ASCII formatted sequence listing with a file name “ROSONC00002USCIP.TXT”, creation date of Dec. 15, 2009, and a size of 1,909 KB. This sequence listing submitted via EFS-Web is part of the specification and is herein incorporated by reference in its entirety.

### BACKGROUND OF THE INVENTION

Upon Wnt receptor activation, three different signaling cascades are activated (Huelsken and Birchmeier, 2001, Curr. Opin. Genet. Dev. 11:547-553): 1) the Wnt/Ca²⁺ pathway, which leads to activation of the protein kinase C and the Ca²⁺ calmodulin dependent protein kinase II; 2) the cytoskeleton pathway, which regulates organization and formation of the cytoskeleton and planar cell polarity; and 3) the canonical Wnt pathway, which controls the intracellular level of the proto-oncoprotein β-catenin. The canonical pathway, also known as the “Wnt/β-catenin” pathway or “β-catenin” pathway is the most studied and the best understood among the three Wnt pathways (Clevers, 2006, Cell 127:469-480).

The key protein of the Wnt/β-catenin pathway is the proto-oncoprotein β-catenin, which can switch between two different intracellular pools. In the absence of the Wnt signal, β-catenin is bound to the cytoplasmic domain of the membrane anchored E-cadherin where it forms together with α-cadherin a connecting bridge to the cytoskeletal protein actin. The β-catenin level in the cytosol is kept low by the so-called destruction complex, which is formed by the active serine-threonin kinase glycogen synthase kinase-3β (GSK3β) and several other cytosolic proteins including the tumor suppressor proteins APC (Adenomatous Polyposis coli) and Axin/Conductin. Phosphorylation of β-catenin by GSK3β leads to its ubiquitinylation via β-TrCP (β-Transducin repeat Containing Protein) and to its degradation by the proteasomal degradation machinery. The activation of the Wnt/β-catenin pathway begins with the hetero-dimerization of the Wnt receptor Frizzled (Fz) with its co-receptor LRP5/6 (low-density lipoprotein receptor related protein). The subsequent hyperphosphorylation of Dishevelled (Dsh) by the activated casein kinase 2 (CK2) leads to the inhibition of GSK3β (Willert et al., 1997, EMBO J. 16:3089-3096). As a consequence, the destruction complex disassembles, β-catenin is not phosphorylated any more, and the level of cytosolic and nuclear β-catenin increases. Nuclear β-catenin interacts with T-cell factor/Lymphoid enhancer factor (TCF/Lef) and displaces co-repressors (Staal et al., 2002, EMBO Rep. 3:63-68). The β-catenin/TCF complex activates transcription of many different target genes.

Products of Wnt target genes unfold a large variety of biochemical functions including cell cycle kinase regulation, cell adhesion, hormone signaling, and transcription regulation. The plurality and diversity of the biochemical functions reflect the variety of different biological effects of the Wnt/β-catenin pathway, including activation of cell-cycle progression and proliferation, inhibition of apoptosis, regulation of embryonic development, cell differentiation, cell growth, and cell migration (reviewed in Vlad et al., 2008, Cellular Signaling 20:795-802). Numerous target genes of the β-catenin/TCF complex have been identified and may be found on the Wnt homepage (http://www.stanford.edu/˜musse/wntwindow.html).

Wnt/β-catenin signaling is involved in adult tissue self-renewal. The Wnt/β-catenin cascade may be required for establishment of the progenitor compartment in the intestinal epithelium (Korinek et al., 1998, Nat. Genet. 19:1-5). Wnt proteins also promote the terminal differentiation of Paneth cells at the base of the intestinal crypts (Van Es et al., 2005, Nature 435:959-963). Wnt/β-catenin signaling is required for the establishment of the hair follicle (van Genderen et al., 1994, Genes Dev. 8:2691-2703) Wnt/β-catenin signals in hair follicles activates bulge stem cells, promotes entry into the hair lineage, and recruits the cells to the transit-amplifying matrix compartment (Lowry et al., 2005, 19:1596-1611; Huelsken et al., 2001, 105:533-545). The Wnt/β-catenin pathway is also an important regulator of hematopoietic stem and progenitor cells and bone homeostasis (reviewed in Clevers, 2006, Cell 469-480).

Wnt/β-catenin signaling is also implicated in cancer. Germline APC mutation is the genetic cause of Familiar Adenomatous Polyposis (FAP) (Kinzler et al., 1991; Nishisho et al., 1991). Loss of both APC alleles occurs in a large majority of sporadic colorectal cancers. β-catenin is inappropriately stabilized as a consequence of the loss of APC (Rubinfeld et al., 1996). These mutations activate β-catenin signaling, inhibit cellular differentiation, increase cellular proliferation, and ultimately result in the formation of precancerous intestinal polyps (Gregorieff and Clevers, 2005, Genes Dev. 19:877-890; Logan and Nusse, 2004, Annu. Rev. Cell Dev. Biol. 20:781-810). In rare cases of colorectal cancer where APC is not mutated, Axin 2 is mutant (Liu et al., 2000), or β-catenin has an activating point mutation that removes its N-terminal Ser/Thr destruction motif (Morin et al., 1997). Activating Wnt/β-catenin pathway mutations are not limited to intestinal cancer. Loss-of-function Axin mutations have also been found in hepatocellular carcinomas, and oncogenic β-catenin mutations occur in a wide variety of solid tumors (reviewed in Reya and Clevers, 2005). Mutational activation of the Wnt/β-catenin cascade may also be involved in hair follicle tumors (reviewed in Clevers, 2006, Cell 469-480). Inactivating mutations in the Wnt/β-catenin signaling pathway have also been identified in human sebaceous tumors, which carried LEF1 mutations (Takeda et al, 2006). Wnt/β-catenin signaling is also implicated in cancer stem cell regulation (Malanchi et al., 2008, 452:650-653; reviewed by Fodde and Brabletz, 2007, Curr. Opin. Cell Biol. 19:150-158).

The identification of patient subpopulations most likely to respond to therapy is a central goal of modern molecular medicine. This notion is particularly important for cancer due to the large number of approved and experimental therapies (Rothenberg et al., 2003, Nat. Rev. Cancer 3:303-309), low response rates to many current treatments, and clinical importance of using the optimal therapy in the first treatment cycle (Dracopoli, 2005, Curr. Mol. Med. 5:103-110). In addition, the narrow therapeutic index and severe toxicity profiles associated with currently marketed cytotoxics results in a pressing need for accurate response prediction. Although recent studies have identified gene expression signatures associated with response to cytotoxic chemotherapies (Folgueria et al., 2005, Clin. Cancer Res. 11:7434-7443; Ayers et al., 2004, 22:2284-2293; Chang et al., 2003, Lancet 362:362-369; Rouzier et al., 2005, Proc. Natl. Acad. Sci. USA 102: 8315-8320), these examples (and others from the literature) remain unvalidated and have not yet had a major effect on clinical practice. In addition to technical issues, such as lack of a standard technology platform and difficulties surrounding the collection of clinical samples, the myriad of cellular processes affected by cytotoxic chemotherapies may hinder the identification of practical and robust gene expression predictors of response to these agents. One exception may be the recent finding by microarray that low mRNA expression of the microtubule-associate protein Tau is predictive of improved response to paclitaxel (Rouzier et al., supra).

To improve on the limitations of cytotoxic chemotherapies, current approaches to drug design in oncology are aimed at modulating specific cell signaling pathways important for tumor growth and survival (Hahn and Weinberg, 2002, Nat. Rev. Cancer 2:331-341; Hanahan and Weinberg, 2000, Cell 100:57-70; Trosko et al., 2004, Ann. N.Y. Acad. Sci. 1028:192-201). In cancer cells, these pathways become deregulated resulting in aberrant signaling, inhibition of apoptosis, increased metastasis, and increased cell proliferation (reviewed in Adjei and Hildalgo, 2005, J. Clin. Oncol. 23:5386-5403). Although normal cells integrate multiple signaling pathways for controlled growth and proliferation, tumors seem to be heavily reliant on activation of one or two pathways (“oncogene activation”). Aberrant Wnt/β-catenin pathway signaling can cause cancer and a number of genetic defects in this pathway may contribute to tumor promotion and progression (reviewed in Polakis, 2000, Genes Dev. 14:1837-1851). Hyperactivation of the Wnt/β-catenin pathway is one of the most frequent signaling abnormalities in several human cancers, including colorectal carcinomas (Morin et al., 1997, Science 275:1787-1790), melanomas (Rubinfeld et al., 1997, Science 275:1790-1792), hepatoblastomas (Koch et al., 1999, Cancer Res. 59:269-273), medulloblastomas (Zurawel et al, 1998, Cancer Res. 58:896-899), prostatic carcinomas (Voeller et al, 1998, Cancer Res. 58:2520-2523), and uterine and ovarian endometrioid adenocarcinomas (Schlosshauer et al., 2000, Mod. Pathol. 13:1066-1071; Mirabelli-Primdahl et al, 1999, Cancer Res. 59:3346-3351; Saegusa and Okayasu, 2001, J. Pathol. 194:59-67; Wu et al., 2001, Cancer Res. 61:8247-8255). Wnt/β-catenin pathway activation is also common in metaplastic carcinomas of the breast (Hayes et al., 2008, Clin. Cancer Res. 14:4038-4044). The components of these aberrant signaling pathways represent attractive selective targets for new anticancer therapies. In addition, responder identification for target therapies may be more achievable than for cytotoxics, as it seems logical that patients with tumors that are “driven” by a particular pathway will respond to therapeutics targeting components of that pathway. Therefore, it is crucial that we develop methods to identify which pathways are active in which tumors and use this information to guide therapeutic decisions. One way to enable this is to identify gene expression profiles that are indicative of pathway activation status.

A multitude of pathway components may activate, modify, or inhibit Wnt/β-catenin signaling at multiple points or may be involved in crosstalk to other pathways. Measuring pathway activity by testing only a few well-characterized pathway components may miss other important pathway mediators. Given its involvement in numerous biological functions and diseases, a gene expression signature-based readout of pathway activation may be more appropriate than relying on a single indicator of pathway activity, as the same signature of gene expression may be elicited by activation of multiple components of the pathway. In addition, by integrating expression data from multiple genes, a quantitative assessment of pathway activity may be possible. In addition to using gene expression signatures for classification cell samples, including but not limited to tumors, by assessing pathway activation status, gene expression signatures for pathway activation may also be used as pharmacodynamic biomarkers, i.e. monitoring pathway inhibition in patient tumors or peripheral tissues post-treatment; as response prediction biomarkers, i.e. prospectively identifying patients harboring tumors that have high levels of a particular pathway activity before treating the patients with inhibitors targeting the pathway; and as early efficacy biomarkers, i.e. an early readout of efficacy. A gene expression signature for pathway activity may also be used to screen for agents that modulate pathway signaling.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. An extensively validated mammalian genome-wide siRNA screen on the Wnt/β-catenin signal transduction pathway. (A) Schematic of the primary, secondary and tertiary phases of the siRNA screen and integration of the data into a global protein-interaction network. (B) Scatter plot of secondary screen data showing normalized luciferase values in DLD1 and SW480 colorectal cancer cell lines. The data represents pooled siRNAs. Colored circles depict siRNA pools that confirmed when deconvoluted to multiple individual siRNAs. (C) Heat map representation of cDNA microarray data for the Wnt/β-catenin gene signature following time-course analysis of siRNA mediated silencing of β-catenin in DLD1 cells. (D) Correct assignment of normal and tumor colon tissue by unsupervised hierarchical clustering using the Wnt/β-catenin gene signature. To show polarity, the expression profile of DLD1 cells transfected with β-catenin siRNAs is shown below the panel.

FIG. 2. The tertiary validation screen employs endogenous β-catenin target genes as an indicator of pathway activation. Heat map representation of gene expression quantification following siRNA transfection in DLD1 cells is shown. For the majority of siRNAs, quantitative PCR was employed to determine the expression of the β-catenin regulated genes following siRNA transfection of DLD1 cells. Additionally, several of the siRNAs were screened by genome-wide microarray analysis of transfected DLD1 cells. Data are log(2) transformed and the average of three independent experiments. The p-value of a siRNA's signature overlap with the WNT/β-catenin signature was calculated using the hypergeometric distribution and Bonferroni corrected.

FIG. 3. Volcano plot of DLD1 genome-scale siRNA primary screen. siRNA pools were colored red if they met both quantitative and statistical thresholds.

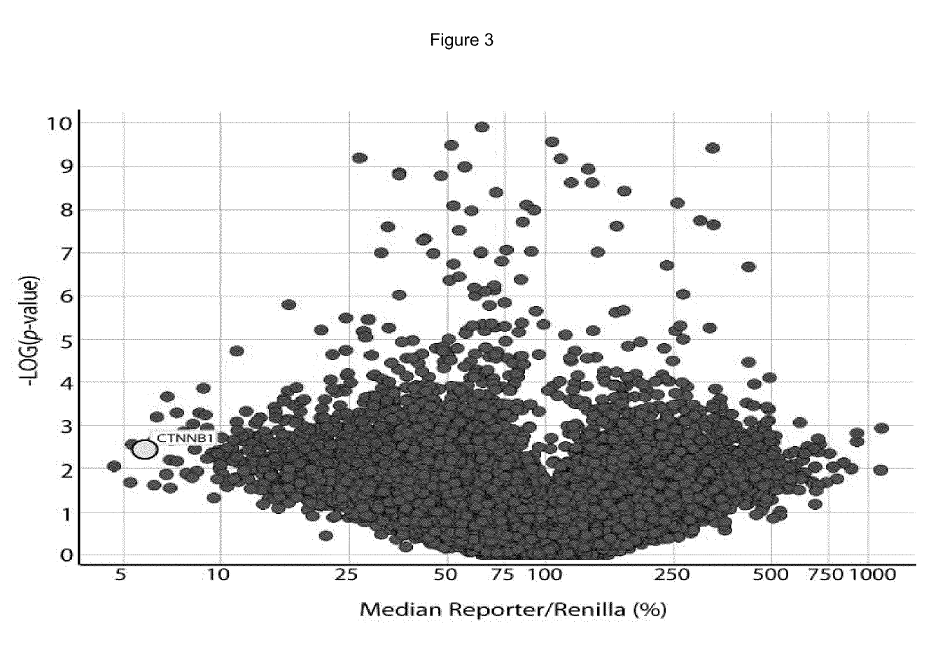

FIG. 4. Determination of the Wnt/β-catenin gene signature. (A) Cartoon schematic of cDNA microarray data reduction. (B) Heat map representation of microarray expression profiles for DLD1 cells transfected with 5 different siRNAs targeting β-catenin. (C) Heat map representation of genes regulated by β-catenin in DLD1 cells and SW480 cells.

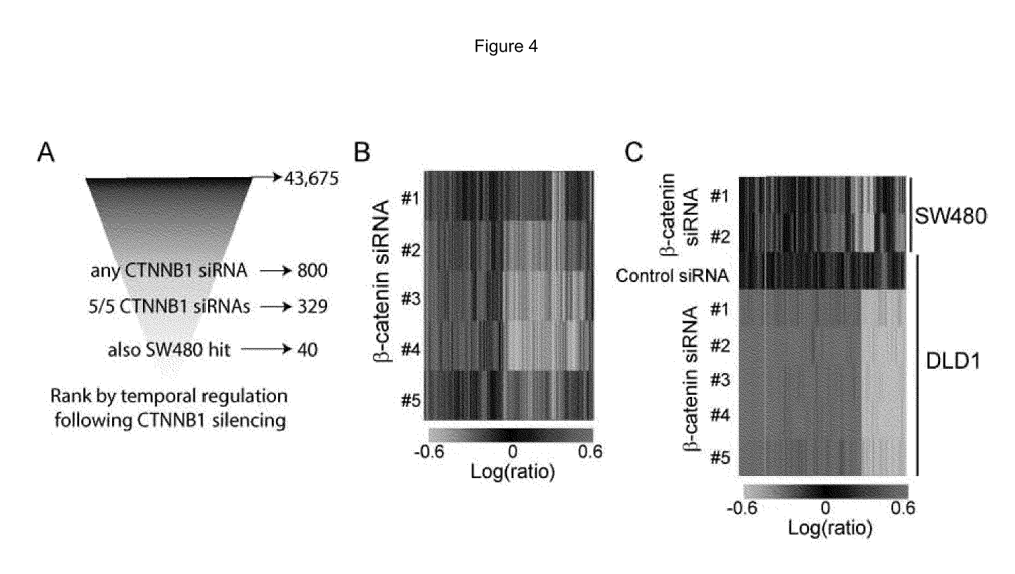

FIG. 5. Heat map of Wnt/β-catenin signaling pathway signature in HCC tumors. Approximately 30% of the patients (boxed) have activated pathway as measured by this set of biomarkers. On the right panel: “0” stands for no-recurrence, and “1” for recurrence.

### DETAILED DESCRIPTION OF THE INVENTION

This section presents a detailed description of the many different aspects and embodiments that are representative of the inventions disclosed herein. This description is by way of several exemplary illustrations, of varying detail and specificity. Other features and advantages of these embodiments are apparent from the additional descriptions provided herein, including the different examples. The provided examples illustrate different components and methodology useful in practicing various embodiments of the invention. The examples are not intended to limit the claimed invention. Based on the present disclosure the ordinary skilled artisan can identify and employ other components and methodology useful for practicing the present invention.

### 3.1 INTRODUCTION

Various embodiments of the invention relate to sets of genetic biomarkers whose expression patterns correlate with an important characteristic of cancer cells, i.e., deregulation of the Wnt/β-catenin signaling pathway. In some embodiments, these sets of biomarkers may be split into two opposing “arms”—the “up” arm (Table 4a), which are the genes that are upregulated, and the “down” arm (Table 4b), which are the genes that are downregulated, as signaling through the Wnt/β-catenin signaling pathway increases. More specifically, some aspects of the invention provide for sets of genetic biomarkers whose expression correlates with the regulation status of the Wnt/β-catenin signaling pathway of a cell sample of a subject, and which can be used to classify samples with deregulated Wnt/β-catenin signaling pathway from samples with regulated Wnt/β-catenin signaling pathway. In a specific embodiment, the cell sample is from a tumor. Wnt/β-catenin signaling pathway regulation status is a useful indicator of the likelihood that a subject will respond to certain therapies, such as inhibitors of the Wnt/β-catenin signaling pathway. Such therapies include, but are not limited to: thiazolidinediones (Wang et al., 2008, J. Surg. Res. Jun. 27, 2008 e-publication ahead of print); PKF115-584 (Doghman et al., 2008, J. Clin. Endocrinol. Metab. E-publication ahead of print, doi: 10.1210/jc.2008-0247); bis[2-(acylamino)phenyl]disulfide (Yamakawa et al., 2008, Biol Pharm. Bull. 31:916-920); FH535 (Handeli and Simon, 2008, Mol. Cancer Ther. 7:521-529); suldinac (Han et al., 2008, Eur. J. Pharmacol. 583:26-31); cyclooxygenase-2 inhibitor celecoxib (Tuynman et al., 2008, Cancer Res. 68:1213-1220); reverse-turn mimetic compounds (U.S. Pat. No. 7,232,822); β-catenin inhibitor compound 1 (WO2005021025); fusicoccin analog (WO2007062243); and FZD10 modulators (WO2008061020). In one aspect of the invention, methods are provided for use of these biomarkers to distinguish between patient groups that will likely respond to inhibitors of the Wnt/β-catenin signaling pathway (predicted responders) and patient groups that will not likely respond to inhibitors of the Wnt/β-catenin signaling pathway and to determine general courses of treatment (predicted non-responders). In another aspect of the invention, methods are provided for use of these biomarkers to classify a cell sample from a subject as having regulated or deregulated Wnt/β-catenin signaling pathway. Another aspect of the invention relates to biomarkers whose expression correlates with a pharmacodynamic effect of a therapeutic agent on the Wnt/β-catenin signaling pathway in subject with cancer. In yet other aspects of the invention, methods are provided for use of these biomarkers to measure the pharmacodynamic effect of a therapeutic agent on the Wnt/β-catenin signaling pathway in a subject with cancer and the use of these biomarkers to rank the efficacy of therapeutic agents to modulate the Wnt/β-catenin signaling pathway. Microarrays comprising these biomarkers are also provided, as well as methods of constructing such microarrays. Each of the biomarkers correspond to a gene in the human genome, i.e., such biomarker is identifiable as all or a portion of a gene. Finally, because each of the above biomarkers correlate with cancer-related conditions, the biomarkers, or the proteins they encode, are likely to be targets for drugs against cancer.

### 3.2 DEFINITIONS

Unless defined otherwise, all technical and scientific terms used herein have the same meaning as commonly understood to one of ordinary skill in the art to which this invention belongs.

As used herein, oligonucleotide sequences that are complementary to one or more of the genes described herein, refers to oligonucleotides that are capable of hybridizing under stringent conditions to at least part of the nucleotide sequence of said genes. Such hybridizable oligonucleotides will typically exhibit at least about 75% sequence identity at the nucleotide level to said genes, preferably about 80% or 85% sequence identity or more preferably about 90% or 95% or more sequence identity to said genes.

“Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence.

The phrase “hybridizing specifically to” refers to the binding, duplexing or hybridizing of a molecule substantially to or only to a particular nucleotide sequence or sequences under stringent conditions when that sequence is present in a complex mixture (e.g., total cellular) DNA or RNA.

“Biomarker” means any gene, protein, or an EST derived from that gene, the expression or level of which changes between certain conditions. Where the expression of the gene correlates with a certain condition, the gene is a biomarker for that condition.

“Biomarker-derived polynucleotides” means the RNA transcribed from a biomarker gene, any cDNA or cRNA produced therefrom, and any nucleic acid derived therefrom, such as synthetic nucleic acid having a sequence derived from the gene corresponding to the biomarker gene.

A gene marker is “informative” for a condition, phenotype, genotype or clinical characteristic if the expression of the gene marker is correlated or anti-correlated with the condition, phenotype, genotype or clinical characteristic to a greater degree than would be expected by chance.

As used herein, the term “gene” has its meaning as understood in the art. However, it will be appreciated by those of ordinary skill in the art that the term “gene” may include gene regulatory sequences (e.g., promoters, enhancers, etc.) and/or intron sequences. It will further be appreciated that definitions of gene include references to nucleic acids that do not encode proteins but rather encode functional RNA molecules such as tRNAs. For clarity, the term gene generally refers to a portion of a nucleic acid that encodes a protein; the term may optionally encompass regulatory sequences. This definition is not intended to exclude application of the term “gene” to non-protein coding expression units but rather to clarify that, in most cases, the term as used in this document refers to a protein coding nucleic acid. In some cases, the gene includes regulatory sequences involved in transcription, or message production or composition. In other embodiments, the gene comprises transcribed sequences that encode for a protein, polypeptide or peptide. In keeping with the terminology described herein, an “isolated gene” may comprise transcribed nucleic acid(s), regulatory sequences, coding sequences, or the like, isolated substantially away from other such sequences, such as other naturally occurring genes, regulatory sequences, polypeptide or peptide encoding sequences, etc. In this respect, the term “gene” is used for simplicity to refer to a nucleic acid comprising a nucleotide sequence that is transcribed, and the complement thereof. In particular embodiments, the transcribed nucleotide sequence comprises at least one functional protein, polypeptide and/or peptide encoding unit. As will be understood by those in the art, this functional term “gene” includes both genomic sequences, RNA or cDNA sequences, or smaller engineered nucleic acid segments, including nucleic acid segments of a non-transcribed part of a gene, including but not limited to the non-transcribed promoter or enhancer regions of a gene. Smaller engineered gene nucleic acid segments may express, or may be adapted to express using nucleic acid manipulation technology, proteins, polypeptides, domains, peptides, fusion proteins, mutants and/or such like. The sequences which are located 5′ of the coding region and which are present on the mRNA are referred to as 5′ untranslated sequences (“5′UTR”). The sequences which are located 3′ or downstream of the coding region and which are present on the mRNA are referred to as 3′ untranslated sequences, or (“3′UTR”).

“Signature” refers to the differential expression pattern. It could be expressed as the number of individual unique probes whose expression is detected when a cRNA product is used in microarray analysis. A signature may be exemplified by a particular set of biomarkers.

A “similarity value” is a number that represents the degree of similarity between two things being compared. For example, a similarity value may be a number that indicates the overall similarity between a cell sample expression profile using specific phenotype-related biomarkers and a control specific to that template (for instance, the similarity to a “deregulated Wnt/β-catenin signaling pathway” template, where the phenotype is deregulated Wnt/β-catenin signaling pathway status). The similarity value may be expressed as a similarity metric, such as a correlation coefficient, or may simply be expressed as the expression level difference, or the aggregate of the expression level differences, between a cell sample expression profile and a baseline template.

As used herein, the terms “measuring expression levels,” “obtaining expression level,” and “detecting an expression level” and the like, includes methods that quantify a gene expression level of, for example, a transcript of a gene, or a protein encoded by a gene, as well as methods that determine whether a gene of interest is expressed at all. Thus, an assay which provides a “yes” or “no” result without necessarily providing quantification, of an amount of expression is an assay that “measures expression” as that term is used herein. Alternatively, a measured or obtained expression level may be expressed as any quantitative value, for example, a fold-change in expression, up or down, relative to a control gene or relative to the same gene in another sample, or a log ratio of expression, or any visual representation thereof, such as, for example, a “heatmap” where a color intensity is representative of the amount of gene expression detected. The genes identified as being differentially expressed in tumor cells having Wnt/β-catenin signaling pathway deregulation may be used in a variety of nucleic acid or protein detection assays to detect or quantify the expression level of a gene or multiple genes in a given sample. Exemplary methods for detecting the level of expression of a gene include, but are not limited to, Northern blotting, dot or slot blots, reporter gene matrix (see for example, U.S. Pat. No. 5,569,588) nuclease protection, RT-PCR, microarray profiling, differential display, 2D gel electrophoresis, SELDI-TOF, ICAT, enzyme assay, antibody assay, and the like.

A “patient” can mean either a human or non-human animal, preferably a mammal.

As used herein, “subject”, as refers to an organism or to a cell sample, tissue sample or organ sample derived therefrom, including, for example, cultured cell lines, biopsy, blood sample, or fluid sample containing a cell. In many instances, the subject or sample derived therefrom, comprises a plurality of cell types. In one embodiment, the sample includes, for example, a mixture of tumor and normal cells. In one embodiment, the sample comprises at least 10%, 15%, 20%, et seq., 90%, or 95% tumor cells. The organism may be an animal, including but not limited to, an animal, such as a cow, a pig, a mouse, a rat, a chicken, a cat, a dog, etc., and is usually a mammal, such as a human.

As used herein, the term “pathway” is intended to mean a set of system components involved in two or more sequential molecular interactions that result in the production of a product or activity. A pathway can produce a variety of products or activities that can include, for example, intermolecular interactions, changes in expression of a nucleic acid or polypeptide, the formation or dissociation of a complex between two or more molecules, accumulation or destruction of a metabolic product, activation or deactivation of an enzyme or binding activity. Thus, the term “pathway” includes a variety of pathway types, such as, for example, a biochemical pathway, a gene expression pathway, and a regulatory pathway. Similarly, a pathway can include a combination of these exemplary pathway types.

“Wnt signaling pathway,” also known as the “Wnt/β-catenin signaling pathway” or “β-catenin signaling pathway” refers to one of the intracellular signaling pathways activated upon Wnt receptor activation, the canonical Wnt/β-catenin signaling pathway, which controls the intracellular level of the proto-oncoproteinβ-catenin. On activation of the Wnt signaling pathway by binding with the Wnt ligand (including, but not limited to, Wnt1, Wnt2, Wnt2B/13, Wnt3, Wnt3A, Wnt4, Wnt5A, Wnt5B, Wnt6, Wnt7A, Wnt8A, Wnt8B, Wnt9A, Wnt9B, Wnt10A, Wnt10B, Wnt11, and Wnt16), the Wnt receptor, Frizzled, hetero-dimerizes with its co-receptor LRP5/6 (low-density lipoprotein receptor related protein). Subsequently, activated Casein kinase 2 hyperphosphorylates Dishevelled, leading to the inhibition of GSK3β, a component of the destruction complex (including APC and Axin/Conductin) which regulates β-catenin levels in the cytosol. Phosphorylation of fβ-catenin by GSK3β leads to its ubiquitinylation via β-TrCP and to its degradation by the proteasomal degradation machinery. As a result of GSK3β inhibition, the destruction complex dissembles, β-catenin is no longer phosphorylated, and the level of cytosolic and nuclear β-catenin increases. Nuclear β-catenin interacts with T-cell factor/Lymphoid enhancer factor (TCF/Lef) and displaces co-repressors. The β-catenin/TCF complex activates transcription of many different target genes. (See also Clevers, 2006, Cell 127:469-480 for a review of the Wnt/β-catenin signaling cascade). The Wnt/β-catenin signaling pathway includes, but is not limited to, the genes, and proteins encoded thereby, listed in Table 1.

TABLE 1
Representative Wnt/β-catenin signaling pathway genes

| Symbol | NCBI Reference Transcript | SEQ ID NO: |
|---|---|---|
| ACTA1 | NM_001100 | SEQ ID NO: 1 |
| ACTA2 | NM_001613 | SEQ ID NO: 2 |
| ACTB | NM_001101 | SEQ ID NO: 3 |
| ACTC1 | NM_005159 | SEQ ID NO: 4 |
| ACTG1 | NM_001614 | SEQ ID NO: 5 |
| ACTG2 | NM_001615 | SEQ ID NO: 6 |
| ACTN1 | NM_001102 | SEQ ID NO: 7 |
| ACTR2 | NM_001005386 | SEQ ID NO: 8 |
| ACTR3 | NM_005721 | SEQ ID NO: 9 |
| ACTR3B | NM_020445 | SEQ ID NO: 10 |
| ACVR1 | NM_001105 | SEQ ID NO: 11 |
| ACVR1B | NM_004302 | SEQ ID NO: 12 |
| ACVR1C | NM_145259 | SEQ ID NO: 13 |
| ACVR2A | NM_001616 | SEQ ID NO: 14 |
| ACVR2B | NM_001106 | SEQ ID NO: 15 |
| AES | NM_198969 | SEQ ID NO: 16 |
| AKT1 | NM_001014432 | SEQ ID NO: 17 |
| AKT2 | NM_001626 | SEQ ID NO: 18 |
| AKT3 | NM_181690 | SEQ ID NO: 19 |
| APC | NM_000038 | SEQ ID NO: 20 |
| APC2 | NM_005883 | SEQ ID NO: 21 |
| ARPC1A | NM_006409 | SEQ ID NO: 22 |
| ARPC1B | NM_005720 | SEQ ID NO: 23 |
| ARPC2 | NM_152862 | SEQ ID NO: 24 |
| ARPC3 | NM_005719 | SEQ ID NO: 25 |
| ARPC4 | NM_005718 | SEQ ID NO: 26 |
| ARPC5 | NM_005717 | SEQ ID NO: 27 |
| AXIN1 | NM_003502 | SEQ ID NO: 28 |
| AXIN2 | NM_004655 | SEQ ID NO: 29 |
| B2M | NM_004048 | SEQ ID NO: 30 |
| BCAR1 | NM_014567 | SEQ ID NO: 31 |
| BCL9 | NM_004326 | SEQ ID NO: 32 |
| BIRC4 | NM_001167 | SEQ ID NO: 33 |
| BIRC5 | NM_001168 | SEQ ID NO: 34 |
| BMP4 | NM_130850 | SEQ ID NO: 35 |
| BTRC | NM_003939 | SEQ ID NO: 36 |
| CACYBP | NM_001007214 | SEQ ID NO: 37 |
| CAMK2A | NM_015981 | SEQ ID NO: 38 |
| CAMK2B | NM_172081 | SEQ ID NO: 39 |
| CAMK2D | NM_172128 | SEQ ID NO: 40 |
| CAMK2G | NM_172170 | SEQ ID NO: 41 |
| CASP9 | NM_032996 | SEQ ID NO: 42 |
| CAV1 | NM_001753 | SEQ ID NO: 43 |
| CBY1 | NM_001002880 | SEQ ID NO: 44 |
| CCND1 | NM_053056 | SEQ ID NO: 45 |
| CCND2 | NM_001759 | SEQ ID NO: 46 |
| CCND3 | NM_001760 | SEQ ID NO: 47 |
| CD44 | NM_001001389 | SEQ ID NO: 48 |
| CDC42 | NM_001791 | SEQ ID NO: 49 |
| CDH1 | NM_004360 | SEQ ID NO: 50 |
| CDH2 | NM_001792 | SEQ ID NO: 51 |
| CDH3 | NM_001793 | SEQ ID NO: 52 |
| CDH5 | NM_001795 | SEQ ID NO: 53 |
| CDKN1A | NM_078467 | SEQ ID NO: 54 |
| CDKN2A | NM_058195 | SEQ ID NO: 55 |
| CDKN2B | NM_004936 | SEQ ID NO: 56 |
| CER1 | NM_005454 | SEQ ID NO: 57 |
| CFL1 | NM_005507 | SEQ ID NO: 58 |
| CFL2 | NM_138638 | SEQ ID NO: 59 |
| CHD8 | NM_020920 | SEQ ID NO: 60 |
| CHUK | NM_001278 | SEQ ID NO: 61 |
| CLDN1 | NM_021101 | SEQ ID NO: 62 |
| COL4A1 | NM_001845 | SEQ ID NO: 63 |
| COL4A2 | NM_001846 | SEQ ID NO: 64 |
| COL4A3 | NM_031362 | SEQ ID NO: 65 |
| COL4A4 | NM_000092 | SEQ ID NO: 66 |
| COL4A5 | NM_033380 | SEQ ID NO: 67 |
| COL4A6 | NM_001847 | SEQ ID NO: 68 |
| CREBBP | NM_001079846 | SEQ ID NO: 69 |
| CRK | NM_005206 | SEQ ID NO: 70 |
| CSNK1A1 | NM_001025105 | SEQ ID NO: 71 |
| CSNK1A1L | NM_145203 | SEQ ID NO: 72 |
| CSNK1D | NM_001893 | SEQ ID NO: 73 |
| CSNK1E | NM_001894 | SEQ ID NO: 74 |
| CSNK1G1 | NM_022048 | SEQ ID NO: 75 |
| CSNK1G2 | NM_001319 | SEQ ID NO: 76 |
| CSNK1G3 | NM_001031812 | SEQ ID NO: 77 |
| CSNK2A1 | NM_177559 | SEQ ID NO: 78 |
| CSNK2A2 | NM_001896 | SEQ ID NO: 79 |
| CSNK2B | NM_001320 | SEQ ID NO: 80 |
| CTBP1 | NM_001328 | SEQ ID NO: 81 |
| CTBP2 | NM_001083914 | SEQ ID NO: 82 |
| CTNNB1 | NM_001098209 | SEQ ID NO: 83 |
| CTNNBIP1 | NM_020248 | SEQ ID NO: 84 |
| CUL1 | NM_003592 | SEQ ID NO: 85 |
| CXXC4 | NM_025212 | SEQ ID NO: 86 |
| DAAM1 | NM_014992 | SEQ ID NO: 87 |
| DAAM2 | NM_015345 | SEQ ID NO: 88 |
| DAB2 | NM_001343 | SEQ ID NO: 89 |
| DIXDC1 | NM_001037954 | SEQ ID NO: 90 |
| DKK1 | NM_012242 | SEQ ID NO: 91 |
| DKK2 | NM_014421 | SEQ ID NO: 92 |
| DKK3 | NM_013253 | SEQ ID NO: 93 |
| DKK4 | NM_014420 | SEQ ID NO: 94 |
| DKKL1 | NM_014419 | SEQ ID NO: 95 |
| DKKL2 | NM_005524.2 | SEQ ID NO: 96 |
| DOCK1 | NM_001380 | SEQ ID NO: 97 |
| DSTN | NM_001011546 | SEQ ID NO: 98 |
| DVL1 | NM_182779 | SEQ ID NO: 99 |
| DVL1L1 | Uncharacterized | SEQ ID NO: 100 |
| DVL2 | NM_004422 | SEQ ID NO: 101 |
| DVL3 | NM_004423 | SEQ ID NO: 102 |
| EIF4E | NM_001968 | SEQ ID NO: 103 |
| EIF4EBP1 | NM_004095 | SEQ ID NO: 104 |
| ENC1 | NM_003633 | SEQ ID NO: 105 |
| EP300 | NM_001429 | SEQ ID NO: 106 |
| FBXW11 | NM_012300 | SEQ ID NO: 107 |
| FBXW2 | NM_012164 | SEQ ID NO: 108 |
| FBXW4 | NM_022039 | SEQ ID NO: 109 |
| FGF4 | NM_002007 | SEQ ID NO: 110 |
| FN1 | NM_212478 | SEQ ID NO: 111 |
| FOSL1 | NM_005438 | SEQ ID NO: 112 |
| FOXN1 | NM_003593 | SEQ ID NO: 113 |
| FOXO3 | NM_201559 | SEQ ID NO: 114 |
| FRAP1 | NM_004958 | SEQ ID NO: 115 |
| FRAT1 | NM_005479 | SEQ ID NO: 116 |
| FRAT2 | NM_012083 | SEQ ID NO: 117 |
| FRZB | NM_001463 | SEQ ID NO: 118 |
| FSHB | NM_001018080 | SEQ ID NO: 119 |
| FZD1 | NM_003505 | SEQ ID NO: 120 |
| FZD10 | NM_007197 | SEQ ID NO: 121 |
| FZD2 | NM_001466 | SEQ ID NO: 122 |
| FZD3 | NM_017412 | SEQ ID NO: 123 |
| FZD4 | NM_012193 | SEQ ID NO: 124 |
| FZD5 | NM_003468 | SEQ ID NO: 125 |
| FZD6 | NM_003506 | SEQ ID NO: 126 |
| FZD7 | NM_003507 | SEQ ID NO: 127 |
| FZD8 | NM_031866 | SEQ ID NO: 128 |
| FZD9 | NM_003508 | SEQ ID NO: 129 |
| GAPDH | NM_002046 | SEQ ID NO: 130 |
| GAST | NM_000805 | SEQ ID NO: 131 |
| GJA1 | NM_000165 | SEQ ID NO: 132 |
| GNAO1 | NM_020988 | SEQ ID NO: 133 |
| GNAQ | NM_002072 | SEQ ID NO: 134 |
| GRB2 | NM_002086 | SEQ ID NO: 135 |
| GSK3A | NM_019884 | SEQ ID NO: 136 |
| GSK3B | NM_002093 | SEQ ID NO: 137 |
| HDAC1 | NM_004964 | SEQ ID NO: 138 |
| HNF1A | NM_000545 | SEQ ID NO: 139 |
| HRAS | NM_005343 | SEQ ID NO: 140 |
| HSP90AB1 | NM_007355 | SEQ ID NO: 141 |
| IKBKB | NM_001556 | SEQ ID NO: 142 |
| ILK | NM_001014794 | SEQ ID NO: 143 |
| ITGAD | NM_005353 | SEQ ID NO: 144 |
| ITGAL | NM_002209 | SEQ ID NO: 145 |
| ITGAM | NM_000632 | SEQ ID NO: 146 |
| ITGAW | Uncharacterized | |
| ITGAX | NM_000887 | SEQ ID NO: 147 |
| ITGB2 | NM_000211 | SEQ ID NO: 148 |
| JUN | NM_002228 | SEQ ID NO: 149 |
| KDR | NM_002253 | SEQ ID NO: 150 |
| KREMEN1 | NM_001039571 | SEQ ID NO: 151 |
| KREMEN2 | NM_172229 | SEQ ID NO: 152 |
| LAMA1 | NM_005559 | SEQ ID NO: 153 |
| LAMB1 | NM_002291 | SEQ ID NO: 154 |
| LAMC1 | NM_002293 | SEQ ID NO: 155 |
| LEF1 | NM_016269 | SEQ ID NO: 156 |
| LIMK2 | NM_005569 | SEQ ID NO: 157 |
| LRP1 | NM_002332 | SEQ ID NO: 158 |
| LRP5 | NM_002335 | SEQ ID NO: 159 |
| LRP6 | NM_002336 | SEQ ID NO: 160 |
| MAP2K1 | NM_002755 | SEQ ID NO: 161 |
| MAP2K2 | NM_030662 | SEQ ID NO: 162 |
| MAP2K3 | NM_145109 | SEQ ID NO: 163 |
| MAP3K11 | NM_002419 | SEQ ID NO: 164 |
| MAP3K7 | NM_003188 | SEQ ID NO: 165 |
| MAP3K7IP1 | NM_153497 | SEQ ID NO: 166 |
| MAP4K1 | NM_007181 | SEQ ID NO: 167 |
| MAPK1 | NM_002745 | SEQ ID NO: 168 |
| MAPK10 | NM_002753 | SEQ ID NO: 169 |
| MAPK11 | NM_002751 | SEQ ID NO: 170 |
| MAPK12 | NM_002969 | SEQ ID NO: 171 |
| MAPK13 | NM_002754 | SEQ ID NO: 172 |
| MAPK14 | NM_001315 | SEQ ID NO: 173 |
| MAPK3 | NM_001040056 | SEQ ID NO: 174 |
| MAPK8 | NM_002750 | SEQ ID NO: 175 |
| MAPK9 | NM_139068 | SEQ ID NO: 176 |
| MARK2 | NM_001039468 | SEQ ID NO: 177 |
| MARK4 | NM_031417 | SEQ ID NO: 178 |
| MDM2 | NM_002392 | SEQ ID NO: 179 |
| MESDC2 | NM_015154 | SEQ ID NO: 180 |
| MKNK1 | NM_003684 | SEQ ID NO: 181 |
| MMP13 | NM_002427 | SEQ ID NO: 182 |
| MMP26 | NM_021801 | SEQ ID NO: 183 |
| MMP7 | NM_002423 | SEQ ID NO: 184 |
| MRCL3 | NM_006471 | SEQ ID NO: 185 |
| MYC | NM_002467 | SEQ ID NO: 186 |
| MYL1 | NM_079420 | SEQ ID NO: 187 |
| MYL2 | NM_000432 | SEQ ID NO: 188 |
| MYL3 | NM_000258 | SEQ ID NO: 189 |
| MYL4 | NM_001002841 | SEQ ID NO: 190 |
| MYL5 | NM_002477 | SEQ ID NO: 191 |
| MYL6 | NM_021019 | SEQ ID NO: 192 |
| MYL6B | NM_002475 | SEQ ID NO: 193 |
| MYL7 | NM_021223 | SEQ ID NO: 194 |
| MYL9 | NM_006097 | SEQ ID NO: 195 |
| MYLK | NM_053026 | SEQ ID NO: 196 |
| MYLK2 | NM_033118 | SEQ ID NO: 197 |
| MYLK3 | NM_182493 | SEQ ID NO: 198 |
| NCL | NM_005381 | SEQ ID NO: 199 |
| NFAT5 | NM_138714 | SEQ ID NO: 200 |
| NFATC1 | NM_172388 | SEQ ID NO: 201 |
| NFATC2 | NM_173091 | SEQ ID NO: 202 |
| NFATC3 | NM_173165 | SEQ ID NO: 203 |
| NFATC4 | NM_004554 | SEQ ID NO: 204 |
| NKD1 | NM_033119 | SEQ ID NO: 205 |
| NKD2 | NM_033120 | SEQ ID NO: 206 |
| NLK | NM_016231 | SEQ ID NO: 207 |
| NRCAM | NM_001037132 | SEQ ID NO: 208 |
| PAK1 | NM_002576 | SEQ ID NO: 209 |
| PIK3CA | NM_006218 | SEQ ID NO: 210 |
| PIK3CB | NM_006219 | SEQ ID NO: 211 |
| PIK3CD | NM_005026 | SEQ ID NO: 212 |
| PIK3R1 | NM_181523 | SEQ ID NO: 213 |
| PIK3R2 | NM_005027 | SEQ ID NO: 214 |
| PIK3R3 | NM_003629 | SEQ ID NO: 215 |
| PIN1 | NM_006221 | SEQ ID NO: 216 |
| PITX2 | NM_153426 | SEQ ID NO: 217 |
| PLAT | NM_000930 | SEQ ID NO: 218 |
| PLAU | NM_002658 | SEQ ID NO: 219 |
| PLAUR | NM_001005376 | SEQ ID NO: 220 |
| PLCB1 | NM_182734 | SEQ ID NO: 221 |
| PLCB2 | NM_004573 | SEQ ID NO: 222 |
| PLCB3 | NM_000932 | SEQ ID NO: 223 |
| PLCB4 | NM_182797 | SEQ ID NO: 224 |
| PLG | NM_000301 | SEQ ID NO: 225 |
| PORCN | NM_203473 | SEQ ID NO: 226 |
| PPARD | NM_006238 | SEQ ID NO: 227 |
| PPM1A | NM_177952 | SEQ ID NO: 228 |
| PPM1J | NM_005167 | SEQ ID NO: 229 |
| PPM1L | NM_139245 | SEQ ID NO: 230 |
| PPP1CB | NM_206876 | SEQ ID NO: 231 |
| PPP1R12A | NM_002480 | SEQ ID NO: 232 |
| PPP2CA | NM_002715 | SEQ ID NO: 233 |
| PPP2CB | NM_001009552 | SEQ ID NO: 234 |
| PPP2R1A | NM_014225 | SEQ ID NO: 235 |
| PPP2R1B | NM_181699 | SEQ ID NO: 236 |
| PPP2R2A | NM_002717 | SEQ ID NO: 237 |
| PPP2R2B | NW_181678 | SEQ ID NO: 238 |
| PPP2R2C | NM_020416 | SEQ ID NO: 239 |
| PPP2R3A | NM_002718 | SEQ ID NO: 240 |
| PPP2R3B | NM_013239 | SEQ ID NO: 241 |
| PPP2R4 | NM_021131 | SEQ ID NO: 242 |
| PPP2R5A | NM_006243 | SEQ ID NO: 243 |
| PPP2R5B | NM_006244 | SEQ ID NO: 244 |
| PPP2R5C | NM_002719 | SEQ ID NO: 245 |
| PPP2R5D | NM_006245 | SEQ ID NO: 246 |
| PPP2R5E | NM_006246 | SEQ ID NO: 247 |
| PPP3CA | NM_000944 | SEQ ID NO: 248 |
| PPP3CB | NM_021132 | SEQ ID NO: 249 |
| PPP3CC | NM_005605 | SEQ ID NO: 250 |
| PPP3R1 | NM_000945 | SEQ ID NO: 251 |
| PPP3R2 | NM_147180 | SEQ ID NO: 252 |
| PRICKLE1 | NM_153026 | SEQ ID NO: 253 |
| PRICKLE2 | NM_198859 | SEQ ID NO: 254 |
| PRKACA | NM_002730 | SEQ ID NO: 255 |
| PRKACB | NM_002731 | SEQ ID NO: 256 |
| PRKACG | NM_002732 | SEQ ID NO: 257 |
| PRKCA | NM_002737 | SEQ ID NO: 258 |
| PRKCB1 | NM_212535 | SEQ ID NO: 259 |
| PRKCG | NM_002739 | SEQ ID NO: 260 |
| PRKX | NM_005044 | SEQ ID NO: 261 |
| PRKY | NM_002760 | SEQ ID NO: 262 |
| PSEN1 | NM_000021 | SEQ ID NO: 263 |
| PTK2 | NM_153831 | SEQ ID NO: 264 |
| PXN | NM_002859 | SEQ ID NO: 265 |
| PYGO1 | NM_015617 | SEQ ID NO: 266 |
| PYGO2 | NM_138300 | SEQ ID NO: 267 |
| RAC1 | NM_006908 | SEQ ID NO: 268 |
| RAC2 | NM_002872 | SEQ ID NO: 269 |
| RAC3 | NM_005052 | SEQ ID NO: 270 |
| RAF1 | NM_002880 | SEQ ID NO: 271 |
| RARA | NM_000964 | SEQ ID NO: 272 |
| RARB | NM_000965 | SEQ ID NO: 273 |
| RARG | NM_000966 | SEQ ID NO: 274 |
| RBX1 | NM_014248 | SEQ ID NO: 275 |
| REST | MM_005612 | SEQ ID NO: 276 |
| RHEB | NM_005614 | SEQ ID NO: 277 |
| RHOA | NM_001664 | SEQ ID NO: 278 |
| RHOU | NM_021205 | SEQ ID NO: 279 |
| ROCK1 | NM_005406 | SEQ ID NO: 280 |
| ROCK2 | NM_004850 | SEQ ID NO: 281 |
| RPS27A | NM_002954 | SEQ ID NO: 282 |
| RPS6KA5 | NM_004755 | SEQ ID NO: 283 |
| RUVBL1 | NM_003707 | SEQ ID NO: 284 |
| RUVBL2 | NM_006666 | SEQ ID NO: 285 |
| SENP2 | NM_021627 | SEQ ID NO: 286 |
| SERPINE1 | NM_000602 | SEQ ID NO: 287 |
| SERPING1 | NM_000062 | SEQ ID NO: 288 |
| SFRP1 | NM_003012 | SEQ ID NO: 289 |
| SFRP2 | NM_003013 | SEQ ID NO: 290 |
| SFRP4 | NM_003014 | SEQ ID NO: 291 |
| SFRP5 | NM_003015 | SEQ ID NO: 292 |
| SHC1 | NM_003029 | SEQ ID NO: 293 |
| SIAH1 | NM_003031 | SEQ ID NO: 294 |
| SKP1A | NM_003197 | SEQ ID NO: 295 |
| SLC9A3R1 | NM_004252 | SEQ ID NO: 296 |
| SMAD2 | NM_001003652 | SEQ ID NO: 297 |
| SMAD3 | NM_005902 | SEQ ID NO: 298 |
| SMAD4 | NM_005359 | SEQ ID NO: 299 |
| SMARCA4 | NM_003072 | SEQ ID NO: 300 |
| SMO | NM_005631 | SEQ ID NO: 301 |
| SOS1 | NM_005633 | SEQ ID NO: 302 |
| SOS2 | NM_006939 | SEQ ID NO: 303 |
| SOX1 | NM_005986 | SEQ ID NO: 304 |
| SOX10 | NM_006941 | SEQ ID NO: 305 |
| SOX11 | NM_003108 | SEQ ID NO: 306 |
| SOX12 | NM_006943 | SEQ ID NO: 307 |
| SOX13 | NM_005686 | SEQ ID NO: 308 |
| SOX14 | NM_004189 | SEQ ID NO: 309 |
| SOX15 | NM_006942 | SEQ ID NO: 310 |
| SOX17 | NM_022454 | SEQ ID NO: 311 |
| SOX18 | NM_018419 | SEQ ID NO: 312 |
| SOX2 | NM_003106 | SEQ ID NO: 313 |
| SOX3 | NM_005634 | SEQ ID NO: 314 |
| SOX4 | NM_003107 | SEQ ID NO: 315 |
| SOX5 | NM_152989 | SEQ ID NO: 316 |
| SOX6 | NM_033326 | SEQ ID NO: 317 |
| SOX7 | NM_031439 | SEQ ID NO: 318 |
| SOX8 | NM_014587 | SEQ ID NO: 319 |
| SOX9 | NM_000346 | SEQ ID NO: 320 |
| SP1 | NM_138473 | SEQ ID NO: 321 |
| SRC | NM_005417 | SEQ ID NO: 322 |
| T | NM_003181 | SEQ ID NO: 323 |
| TBL1X | NM_005647 | SEQ ID NO: 324 |
| TBL1XR1 | NM_024665 | SEQ ID NO: 325 |
| TBL1Y | NM_134258 | SEQ ID NO: 326 |
| TCF3 | NM_003200 | SEQ ID NO: 327 |
| TCF4 | NM_001083962 | SEQ ID NO: 328 |
| TCF7 | NM_003202 | SEQ ID NO: 329 |
| TCF7L1 | NM_031283 | SEQ ID NO: 330 |
| TCF7L2 | NM_030756 | SEQ ID NO: 331 |
| TGFB1 | NM_000660 | SEQ ID NO: 332 |
| TGFB2 | NM_003238 | SEQ ID NO: 333 |
| TGFB3 | NM_003239 | SEQ ID NO: 334 |
| TGFBR1 | NM_004612 | SEQ ID NO: 335 |
| TGFBR2 | NM_003242 | SEQ ID NO: 336 |
| TLE1 | NM_005077 | SEQ ID NO: 337 |
| TLE2 | NM_003260 | SEQ ID NO: 338 |
| TLE3 | NM_005078 | SEQ ID NO: 339 |
| TLE4 | NM_007005 | SEQ ID NO: 340 |
| TLN1 | NM_006289 | SEQ ID NO: 341 |
| TLN2 | NM_015059 | SEQ ID NO: 342 |
| TP53 | NM_000546 | SEQ ID NO: 343 |
| TSC2 | NM_001077183 | SEQ ID NO: 344 |
| UBB | NM_018955 | SEQ ID NO: 345 |
| UBC | NM_021009 | SEQ ID NO: 346 |
| UBD | NM_006398 | SEQ ID NO: 347 |
| VANGL1 | NM_138959 | SEQ ID NO: 348 |
| VANGL2 | NM_020335 | SEQ ID NO: 349 |
| VAV1 | NM_005428 | SEQ ID NO: 350 |
| VCL | NM_003373 | SEQ ID NO: 351 |
| VEGFA | NM_001025368 | SEQ ID NO: 352 |
| VIM | NM_003380 | SEQ ID NO: 353 |
| VTN | NM_000638 | SEQ ID NO: 354 |
| WASL | NM_003941 | SEQ ID NO: 355 |
| WIF1 | NM_007191 | SEQ ID NO: 356 |
| WISP1 | NM_003882 | SEQ ID NO: 357 |
| WISP2 | NM_003881 | SEQ ID NO: 358 |
| WNT1 | NM_005430 | SEQ ID NO: 359 |
| WNT10A | NM_025216 | SEQ ID NO: 360 |
| WNT10B | NM_003394 | SEQ ID NO: 361 |
| WNT11 | NM_004626 | SEQ ID NO: 362 |
| WNT16 | NM_016087 | SEQ ID NO: 363 |
| WNT2 | NM_003391 | SEQ ID NO: 364 |
| WNT2B | NM_004185 | SEQ ID NO: 365 |
| WNT3 | NM_030753 | SEQ ID NO: 366 |
| WNT3A | NM_033131 | SEQ ID NO: 367 |
| WNT4 | NM_030761 | SEQ ID NO: 368 |
| WNT5A | NM_003392 | SEQ ID NO: 369 |
| WNT5B | NM_030775 | SEQ ID NO: 370 |
| WNT6 | NM_006522 | SEQ ID NO: 371 |
| WNT7A | NM_004625 | SEQ ID NO: 372 |
| WNT7B | NM_058238 | SEQ ID NO: 373 |
| WNT8A | NM_058244 | SEQ ID NO: 374 |
| WNT8B | NM_003393 | SEQ ID NO: 375 |
| WNT9A | NM_003395 | SEQ ID NO: 376 |
| WNT9B | NM_003396 | SEQ ID NO: 377 |
| ZFYVE9 | NM_007324 | SEQ ID NO: 378 |

“Wnt/β-catenin agent,” “Wnt agent,” or “β-catenin agent” refers to a drug or agent modulates the canonical Wnt/β-catenin signaling pathway. A Wnt/β-catenin pathway inhibitor inhibits the canonical Wnt/β-catenin pathway signaling. Molecular targets of such agents may include β-catenin, TCF4, APC, axin, GSK3β, and any of the genes listed in Table 1. Such agents are known in the art and include, but are not limited to: thiazolidinediones (Wang et al., 2008, J. Surg. Res. Jun. 27, 2008 e-publication ahead of print); PKF115-584 (Doghman et al., 2008, J. Clin. Endocrinol. Metab. E-publication ahead of print, doi: 10.1210/jc.2008-0247); bis[2-(acylamino)phenyl]disulfide (Yamakawa et al., 2008, Biol Pharm. Bull. 31:916-920); FH535 (Handeli and Simon, 2008, Mol. Cancer Ther. 7:521-529); suldinac (Han et al., 2008, Eur. J. Pharmacol. 583:26-31); cyclooxygenase-2 inhibitor celecoxib (Tuynman et al., 2008, Cancer Res. 68:1213-1220); reverse-turn mimetic compounds (U.S. Pat. No. 7,232,822); β-catenin inhibitor compound 1 (WO2005021025); fusicoccin analog (WO2007062243); and FZD10 modulators (WO2008061020). The siRNA agents against target genes listed in the Examples that passed the tertiary validation screen are also exemplary Wnt/β-catenin pathway agents (see also Table 5).

The term “deregulated Wnt/β-catenin pathway” is used herein to mean that the Wnt/β-catenin signaling pathway is either hyperactivated or hypoactivated. A Wnt/β-catenin signaling pathway is hyperactivated in a sample (for example, a tumor sample) if it has at least 10%, 20%, 30%, 40%, 50%, 75%, 100%, 200%, 500%, 1000% greater activity/signaling than the Wnt/β-catenin signaling pathway in a normal (regulated) sample. A Wnt/β-catenin signaling pathway is hypoactivated if it has at least 10%, 20%, 30%, 40%, 50%, 75%, 100% less activity/signaling in a sample (for example, a tumor sample) than the Wnt/β-catenin signaling pathway in a normal (regulated) sample. The normal sample with the regulated Wnt/β-catenin signaling pathway may be from adjacent normal tissue, may be other tumor samples which do not have deregulated Wnt/β-catenin signaling, or may be a pool of samples. Alternatively, comparison of samples' Wnt/β-catenin signaling pathway status may be done with identical samples which have been treated with a drug or agent vs. vehicle. The change in activation status may be due to a mutation of one or more genes in the Wnt/β-catenin signaling pathway (such as point mutations, deletion, or amplification), changes in transcriptional regulation (such as methylation, phosphorylation, or acetylation changes), or changes in protein regulation (such as translation or post-translational control mechanisms).

The term “oncogenic pathway” is used herein to mean a pathway that when hyperactivated or hypoactivated contributes to cancer initiation or progression. In one embodiment, an oncogenic pathway is one that contains an oncogene or a tumor suppressor gene.

The term “treating” in its various grammatical forms in relation to the present invention refers to preventing (i.e. chemoprevention), curing, reversing, attenuating, alleviating, minimizing, suppressing, or halting the deleterious effects of a disease state, disease progression, disease causative agent (e.g. bacteria or viruses), or other abnormal condition. For example, treatment may involve alleviating a symptom (i.e., not necessarily all the symptoms) of a disease of attenuating the progression of a disease.

“Treatment of cancer,” as used herein, refers to partially or totally inhibiting, delaying, or preventing the progression of cancer including cancer metastasis; inhibiting, delaying, or preventing the recurrence of cancer including cancer metastasis; or preventing the onset or development of cancer (chemoprevention) in a mammal, for example, a human. In addition, the methods of the present invention may be practiced for the treatment of human patients with cancer. However, it is also likely that the methods would also be effective in the treatment of cancer in other mammals.

As used herein, the term “therapeutically effective amount” is intended to qualify the amount of the treatment in a therapeutic regiment necessary to treat cancer. This includes combination therapy involving the use of multiple therapeutic agents, such as a combined amount of a first and second treatment where the combined amount will achieve the desired biological response. The desired biological response is partial or total inhibition, delay, or prevention of the progression of cancer including cancer metastasis; inhibition, delay, or prevention of the recurrence of cancer including cancer metastasis; or the prevention of the onset of development of cancer (chemoprevention) in a mammal, for example, a human.

“Displaying or outputting a classification result, prediction result, or efficacy result” means that the results of a gene expression based sample classification or prediction are communicated to a user using any medium, such as for example, orally, writing, visual display, etc., computer readable medium or computer system. It will be clear to one skilled in the art that outputting the result is not limited to outputting to a user or a linked external component(s), such as a computer system or computer memory, but may alternatively or additionally be outputting to internal components, such as any computer readable medium. Computer readable media may include, but are not limited to hard drives, floppy disks, CD-ROMs, DVDs, DATs. Computer readable media does not include carrier waves or other wave forms for data transmission. It will be clear to one skilled in the art that the various sample classification methods disclosed and claimed herein, can, but need not be, computer-implemented, and that, for example, the displaying or outputting step can be done by, for example, by communicating to a person orally or in writing (e.g., in handwriting).

### 3.3 BIOMARKERS USEFUL IN CLASSIFYING SUBJECTS AND PREDICTING RESPONSE TO THERAPEUTIC AGENTS

### 3.3.1 Biomarker Sets

One aspect of the invention provides a set of 38 biomarkers whose expression is correlated with Wnt/β-catenin signaling pathway deregulation by clustering analysis. These biomarkers identified as useful for classifying subjects according to regulation status of the Wnt/β-catenin signaling pathway, predicting response of a subject to a compound that modulates the Wnt/β-catenin signaling pathway, measuring pharmacodynamic effect on the Wnt/β-catenin signaling pathway of a therapeutic agent, or measuring are listed as SEQ ID NOs: 388-425 (see also Table 3). Another aspect of the invention provides a method of using these biomarkers to distinguish tumor types in diagnosis or to predict response to therapeutic agents. Yet other aspects of the invention provide methods of using these biomarkers as pharmacodynamic biomarkers, i.e. monitoring pathway inhibition in patient tumors or peripheral tissues post-treatment; as response prediction biomarkers, i.e. prospectively identifying patients harboring tumors that have high levels of a particular pathway activity before treating the patients with inhibitors targeting the pathway; and as early efficacy biomarkers, i.e. an early readout of efficacy. In one embodiment of the invention, the 38 biomarker set may be split into two opposing “arms”—the “up” arm (see Table 4a), which are the genes that are upregulated, and the “down” arm (see Table 4b), which are the genes that are downregulated, as signaling through the Wnt/β-catenin pathway increases.

In one embodiment, the invention provides a set of 38 biomarkers that can classify subjects by Wnt/β-catenin signaling pathway regulation status, i.e. distinguish between subjects having regulated and deregulated Wnt/β-catenin signaling pathways. These biomarkers are listed in Table 3. The invention also provides subsets of at least 5, 10, 15, 20, 25, 30, 35 biomarkers, drawn from the set of 38, that can distinguish between subjects having deregulated and regulated Wnt/β-catenin signaling pathways. Alternatively, a subset of at least 3, 5, 10, or 15 biomarkers, drawn from the “up” arm (see Table 4a) and a subset of at least 3, 5, 10, or 15 biomarkers from the “down” arm (see Table 4b) that can distinguish between subjects having deregulated and regulated Wnt/β-catenin signaling pathways. The invention also provides a method of using the above biomarkers to distinguish between subjects having deregulated or regulated Wnt/β-catenin signaling pathway.

In another embodiment, the invention provides a set of 38 biomarkers that can be used to predict response of a subject to a Wnt/β-catenin signaling pathway agent. In a more specific embodiment, the invention provides a subset of at least 5, 10, 15, 20, 25, 30, or 35 biomarkers, drawn from the set of 38, that can be used to predict the response of a subject to an agent that modulates the Wnt/β-catenin signaling pathway. In another embodiment, the invention provides a set of 38 biomarkers that can be used to select a Wnt/β-catenin pathway agent for treatment of a subject with cancer. In a more specific embodiment, the invention provides a subset of at least 5, 10, 15, 20, 25, 30, or 35 biomarkers, drawn from the set of 38 that can be used to select a Wnt/β-catenin pathway agent for treatment of a subject with cancer. Alternatively, a subset of at least 3, 5, 10, or 15 biomarkers, drawn from the “up” arm (see Table 4a) and a subset of at least 3, 5, 10, or 15 biomarkers from the “down” arm (see Table 4b) can be used to predict response of a subject to a Wnt/β-catenin signaling pathway agent or to select a Wnt/β-catenin signaling pathway agent for treatment of a subject with cancer.

In another embodiment, the invention provides a set of 38 genetic biomarkers that can be used to determine whether an agent has a pharmacodynamic effect on the Wnt/β-catenin signaling pathway in a subject. The biomarkers provided may be used to monitor inhibition of the Wnt/β-catenin signaling pathway at various time points following treatment of a subject with said agent. In a more specific embodiment, the invention provides a subset of at least 5, 10, 15, 20, 25, 30, or 35 biomarkers, drawn from the set of 38 that can be used to monitor pharmacodynamic activity of an agent on the Wnt/β-catenin signaling pathway. Alternatively, a subset of at least 3, 5, 10, or 15 biomarkers, drawn from the “up” arm (see Table 4a) and a subset of at least 3, 5, 10, or 15 biomarkers from the “down” arm (see Table 4b) can be used to determine whether an agent has a pharmacodynamic effect on the Wnt/β-catenin signaling pathway or monitor pharmacodynamic activity of an agent on the Wnt/β-catenin signaling pathway.

Any of the sets of biomarkers provided above may be used alone specifically or in combination with biomarkers outside the set. For example, biomarkers that distinguish Wnt/β-catenin pathway regulation status may be used in combination with biomarkers that distinguish growth factor signaling pathway regulation status (see provisional applications by James Watters et al., filed on Mar. 22, 2008, application No. 61/070,368; filed on May 16, 2008, application No. 61/128,001; filed on Jun. 20, 2008, application No. 61/132,649) or p53 functional status (see provisional application, “Gene Expression Signature for Assessing p53 Pathway Functional Status,” by Andrey Loboda et al., filed on Mar. 22, 2008, application No. 61/070,259). Any of the biomarker sets provided above may also be used in combination with other biomarkers for cancer, or for any other clinical or physiological condition.

### 3.3.2 Identification of the Biomarkers

The present invention provides sets of biomarkers for the identification of conditions or indications associated with deregulated Wnt/β-catenin signaling pathway. Generally, the biomarker sets were identified by determining which of ˜44,000 human biomarkers had expression patterns that correlated with the conditions or indications.

In one embodiment, the method for identifying biomarker sets is as follows. After extraction and labeling of target polynucleotides, the expression of all biomarkers (genes) in a sample X is compared to the expression of all biomarkers in a standard or control. In one embodiment, the standard or control comprises target polynucleotides derived from a sample from a normal individual (i.e. an individual not having Wnt/β-catenin pathway deregulation). Alternatively, the standard or control comprises polynucleotides derived from normal tissue adjacent to a tumor or from tumors not having Wnt/β-catenin pathway deregulation. In a preferred embodiment, the standard or control is a pool of target polynucleotide molecules. The pool may be derived from collected samples from a number of normal individuals. In another embodiment, the pool comprises samples taken from a number of individuals with tumors not having Wnt/β-catenin pathway deregulation. In another preferred embodiment, the pool comprises an artificially-generated population of nucleic acids designed to approximate the level of nucleic acid derived from each biomarker found in a pool of biomarker-derived nucleic acids derived from tumor samples. In yet another embodiment, the pool is derived from normal or cancer lines or cell line samples.

The comparison may be accomplished by any means known in the art. For example, expression levels of various biomarkers may be assessed by separation of target polynucleotide molecules (e.g. RNA or cDNA) derived from the biomarkers in agarose or polyacrylamide gels, followed by hybridization with biomarker-specific oligonucleotide probes. Alternatively, the comparison may be accomplished by the labeling of target polynucleotide molecules followed by separation on a sequencing gel. Polynucleotide samples are placed on the gel such that patient and control or standard polynucleotides are in adjacent lanes. Comparison of expression levels is accomplished visually or by means of densitometer. In a preferred embodiment, the expression of all biomarkers is assessed simultaneously by hybridization to a microarray. In each approach, biomarkers meeting certain criteria are identified as associated with tumors having Wnt/β-catenin signaling pathway deregulation.

A biomarker is selected based upon significant difference of expression in a sample as compared to a standard or control condition. Selection may be made based upon either significant up- or down regulation of the biomarker in the patient sample. Selection may also be made by calculation of the statistical significance (i.e., the p-value) of the correlation between the expression of the biomarker and the condition or indication. Preferably, both selection criteria are used. Thus, in one embodiment of the invention, biomarkers associated with deregulation Wnt/β-catenin signaling pathway in a tumor are selected where the biomarkers show both more than two-fold change (increase or decrease) in expression as compared to a standard, and the p-value for the correlation between the existence of Wnt/β-catenin pathway deregulation and the change in biomarker expression is no more than 0.01 (i.e., is statistically significant).

Expression profiles comprising a plurality of different genes in a plurality of N cancer tumor samples can be used to identify markers that correlate with, and therefore are useful for discriminating different clinical categories. In a specific embodiment, a correlation coefficient ρ between a vector \vec{c} representing clinical categories or clinical parameters, e.g., a regulated or deregulated Wnt/β-catenin signaling pathway, in the N tumor samples and a vector \vec{r} representing the measured expression levels of a gene in the N tumor samples is used as a measure of the correlation between the expression level of the gene and Wnt/β-catenin signaling pathway status. The expression levels can be a measured abundance level of a transcript of the gene, or any transformation of the measured abundance, e.g., a logarithmic or a log ratio. Specifically, the correlation coefficient may be calculated as:

ρ=(\vec{c}·\vec{r})/(∥\vec{c}∥·∥\vec{r}∥) (1)

Biomarkers for which the coefficient of correlation exceeds a cutoff are identified as Wnt/β-catenin pathway signaling status-informative biomarkers specific for a particular clinical category, e.g., deregulated Wnt/β-catenin pathway signaling status, within a given patient subset. Such a cutoff or threshold may correspond to a certain significance of the set of obtained discriminating genes. The threshold may also be selected based on the number of samples used. For example, a threshold can be calculated as 3×1/\sqrt{n−3}, where 1/\sqrt{n−3} is the distribution width and the number of samples. In a specific embodiment, markers are chosen if the correlation coefficient is greater than about 0.3 or less than about −0.3.

Next, the significance of the set of biomarker genes can be evaluated. The significance may be calculated by any appropriate statistical method. In a specific example, a Monte-Carlo technique is used to randomize the association between the expression profiles of the plurality of patients and the clinical categories to generate a set of randomized data. The same biomarker selection procedure as used to select the biomarker set is applied to the randomized data to obtain a control biomarker set. A plurality of such runs can be performed to generate a probability distribution of the number of genes in control biomarker sets. In a preferred embodiment, 10,000 such runs are performed. From the probability distribution, the probability of finding a biomarker set consisting of a given number of biomarkers when no correlation between the expression levels and phenotype is expected (i.e., based randomized data) can be determined. The significance of the biomarker set obtained from the real data can be evaluated based on the number of biomarkers in the biomarker set by comparing to the probability of obtaining a control biomarker set consisting of the same number of biomarkers using the randomized data. In one embodiment, if the probability of obtaining a control biomarker set consisting of the same number of biomarkers using the randomized data is below a given probability threshold, the biomarker set is said to be significant.

Once a biomarker set is identified, the biomarkers may be rank-ordered in order of correlation or significance of discrimination. One means of rank ordering is by the amplitude of correlation between the change in gene expression of the biomarker and the specific condition being discriminated. Another, preferred, means is to use a statistical metric. In a specific embodiment, the metric is a t-test-like statistic:

$$t = \frac{( 〈 {x}_{1} 〉 - 〈 {x}_{2} 〉 )}{\sqrt{[ {σ}_{1}^{2} ( {n}_{1} - 1 ) + {σ}_{2}^{2} ( {n}_{2} - 1 ) ] / ( {n}_{1} + {n}_{2} - 1 ) / ( 1 / {n}_{1} + 1 / {n}_{2} )}} \qquad ( 2 )$$

In this equation, [symbol] x₁ [symbol] is the error-weighted average of the log ratio of transcript expression measurements within a first clinical group (e.g., deregulated Wnt/β-catenin pathway signaling), [symbol] x₂ [symbol] is the error-weighted average of log ratio within a second, related clinical group (e.g., regulated Wnt/β-catenin pathway signaling), σ₁ is the variance of the log ratio within the first clinical group (e.g., deregulated Wnt/β-catenin pathway signaling), n₁ is the number of samples for which valid measurements of log ratios are available, σ₂ is the variance of log ratio within the second clinical group (e.g., regulated Wnt/β-catenin pathway signaling), and n₂ is the number of samples for which valid measurements of log ratios are available. The t-value represents the variance-compensated difference between two means. The rank-ordered biomarker set may be used to optimize the number of biomarkers in the set used for discrimination.

A set of genes for Wnt/β-catenin pathway signaling status can also be identified using an iterative approach. This is accomplished generally in a “leave one out” method as follows. In a first run, a subset, for example five, of the biomarkers from the top of the ranked list is used to generate a template, where out of N samples, N−1 are used to generate the template, and the status of the remaining sample is predicted. This process is repeated for every sample until every one of the N samples is predicted once. In a second run, one or more additional biomarkers, for example five additional biomarkers, are added, so that a template is now generated from 10 biomarkers, and the outcome of the remaining sample is predicted. This process is repeated until the entire set of biomarkers is used to generate the template. For each of the runs, type 1 error (false negative) and type 2 errors (false positive) are counted. The set of top-ranked biomarkers that corresponds to lowest type 1 error rate, or type 2 error rate, or preferably the total of type 1 and type 2 error rate is selected.

For Wnt/β-catenin pathway signaling status biomarkers, validation of the marker set may be accomplished by an additional statistic, a survival model. This statistic generates the probability of tumor distant metastases as a function of time since initial diagnosis. A number of models may be used, including Weibull, normal, log-normal, log logistic, log-exponential, or log-Rayleigh (Chapter 12 “Life Testing”, S-PLUS 2000 GUIDE TO STATISTICS, Vol. 2, p. 368 (2000)). For the “normal” model, the probability of distant metastases P at time t is calculated as

P=α×exp(−t²/τ²) (3)

where α is fixed and equal to 1, τ and is a parameter to be fitted and measures the “expected lifetime”.

It is preferable that the above biomarker identification process be iterated one or more times by excluding one or more samples from the biomarker selection or ranking (i.e., from the calculation of correlation). Those samples being excluded are the ones that can not be predicted correctly from the previous iteration. Preferably, those samples excluded from biomarker selection in this iteration process are included in the classifier performance evaluation, to avoid overstating the performance.

Once a set of genes for Wnt/β-catenin pathway signaling status has been identified, the biomarkers may be split into two opposing “arms” —the “up” arm (see Table 4a), which are the genes that are upregulated, and the “down” arm (see Table 4b), which are the genes that are downregulated, as signaling through the Wnt/β-catenin pathway increases.

It will be apparent to those skilled in the art that the above methods, in particular the statistical methods, described above, are not limited to the identification of biomarkers associated with Wnt/β-catenin signaling pathway regulation status, but may be used to identify set of biomarker genes associated with any phenotype. The phenotype can be the presence or absence of a disease such as cancer, or the presence or absence of any identifying clinical condition associated with that cancer. In the disease context, the phenotype may be prognosis such as survival time, probability of distant metastases of disease condition, or likelihood of a particular response to a therapeutic or prophylactic regimen. The phenotype need not be cancer, or a disease; the phenotype may be a nominal characteristic associated with a healthy individual.

### 3.3.3 Sample Collection

In the present invention, target polynucleotide molecules are typically extracted from a sample taken from an individual afflicted with cancer or tumor cell lines, and corresponding normal/control tissues or cell lines, respectively. Samples may also be taken from primary cell lines or ex vivo cultures of cells taken from an animal or patient. The sample may be collected in any clinically acceptable manner, but must be collected such that biomarker-derived polynucleotides (i.e., RNA) are preserved. mRNA or nucleic acids derived therefrom (i.e., cDNA or amplified DNA) are preferably labeled distinguishably from standard or control polynucleotide molecules, and both are simultaneously or independently hybridized to a microarray comprising some or all of the biomarkers or biomarker sets or subsets described above. Alternatively, mRNA or nucleic acids derived therefrom may be labeled with the same label as the standard or control polynucleotide molecules, wherein the intensity of hybridization of each at a particular probe is compared. A sample may comprise any clinically relevant tissue sample, such as a tumor biopsy, fine needle aspirate, or hair follicle, or a sample of bodily fluid, such as blood, plasma, serum, lymph, ascitic fluid, cystic fluid, urine. The sample may be taken from a human, or, in a veterinary context, from non-human animals such as ruminants, horses, swine or sheep, or from domestic companion animals such as felines and canines. Additionally, the samples may be from frozen or archived formalin-fixed, paraffin-embedded (FFPE) tissue samples.

Methods for preparing total and poly(A)+ RNA are well known and are described generally in Sambrook et al., MOLECULAR CLONING—A LABORATORY MANUAL (2ND ED.), Vols. 1-3, Cold Spring Harbor Laboratory, Cold Spring Harbor, N.Y. (1989)) and Ausubel et al., CURRENT PROTOCOLS IN MOLECULAR BIOLOGY, vol. 2, Current Protocols Publishing, New York (1994)).

RNA may be isolated from eukaryotic cells by procedures that involve lysis of the cells and denaturation of the proteins contained therein. Cells of interest include wild-type cells (i.e., non-cancerous), drug-exposed wild-type cells, tumor- or tumor-derived cells, modified cells, normal or tumor cell line cells, and drug-exposed modified cells.

Additional steps may be employed to remove DNA. Cell lysis may be accomplished with a nonionic detergent, followed by microcentrifugation to remove the nuclei and hence the bulk of the cellular DNA. In one embodiment, RNA is extracted from cells of the various types of interest using guanidinium thiocyanate lysis followed by CsCl centrifugation to separate the RNA from DNA (Chirgwin et al., Biochemistry 18:5294-5299 (1979)). Poly(A)+ RNA is selected by selection with oligo-dT cellulose (see Sambrook et al, MOLECULAR CLONING—A LABORATORY MANUAL (2ND ED.), Vols. 1-3, Cold Spring Harbor Laboratory, Cold Spring Harbor, N.Y. (1989). Alternatively, separation of RNA from DNA can be accomplished by organic extraction, for example, with hot phenol or phenol/chloroform/isoamyl alcohol.

If desired, RNase inhibitors may be added to the lysis buffer. Likewise, for certain cell types, it may be desirable to add a protein denaturation/digestion step to the protocol.

For many applications, it is desirable to preferentially enrich mRNA with respect to other cellular RNAs, such as transfer RNA (tRNA) and ribosomal RNA (rRNA). Most mRNAs contain a poly(A) tail at their 3′ end. This allows them to be enriched by affinity chromatography, for example, using oligo(dT) or poly(U) coupled to a solid support, such as cellulose or Sephadex® (see Ausubel et al., CURRENT PROTOCOLS IN MOLECULAR BIOLOGY, vol. 2, Current Protocols Publishing, New York (1994). Once bound, poly(A)+ mRNA is eluted from the affinity column using 2 mM EDTA/0.1% SDS.

The sample of RNA can comprise a plurality of different mRNA molecules, each different mRNA molecule having a different nucleotide sequence. In a specific embodiment, the mRNA molecules in the RNA sample comprise at least 100 different nucleotide sequences. More preferably, the mRNA molecules of the RNA sample comprise mRNA molecules corresponding to each of the biomarker genes. In another specific embodiment, the RNA sample is a mammalian RNA sample.

In a specific embodiment, total RNA or mRNA from cells is used in the methods of the invention. The source of the RNA can be cells of a plant or animal, human, mammal, primate, non-human animal, dog, cat, mouse, rat, bird, yeast, eukaryote, prokaryote, etc. In specific embodiments, the method of the invention is used with a sample containing total mRNA or total RNA from 1×10⁶ cells or less. In another embodiment, proteins can be isolated from the foregoing sources, by methods known in the art, for use in expression analysis at the protein level.

Probes to the homologs of the biomarker sequences disclosed herein can be employed preferably wherein non-human nucleic acid is being assayed.

### 3.4 METHODS OF USING WNT/β-CATENIN PATHWAY DEREGULATION BIOMARKER SETS

### 3.4.1 Diagnostic/Sample Classification Methods

The invention provides for methods of using the biomarker sets to analyze a sample from an individual or subject so as to determine or classify the subject's sample at a molecular level, whether a sample has a deregulated or regulated Wnt/β-catenin pathway. The sample may or may not be derived from a tumor. The individual need not actually be afflicted with cancer. Essentially, the expression of specific biomarker genes in the individual, or a sample taken therefrom, is compared to a standard or control. For example, assume two cancer-related conditions, X and Y. One can compare the level of expression of Wnt/β-catenin pathway biomarkers for condition X in an individual to the level of the biomarker-derived polynucleotides in a control, wherein the level represents the level of expression exhibited by samples having condition X. In this instance, if the expression of the markers in the individual's sample is substantially (i.e., statistically) different from that of the control, then the individual does not have condition X. Where, as here, the choice is bimodal (i.e. a sample is either X or Y), the individual can additionally be said to have condition Y. Of course, the comparison to a control representing condition Y can also be performed. Preferably, both are performed simultaneously, such that each control acts as both a positive and a negative control. The distinguishing result may thus either be a demonstrable difference from the expression levels (i.e. the amount of marker-derived RNA, or polynucleotides derived therefrom) represented by the control, or no significant difference.

Thus, in one embodiment, the method of determining a particular tumor-related status of an individual comprises the steps of (1) hybridizing labeled target polynucleotides from an individual to a microarray containing the above biomarker set or a subset of the biomarkers; (2) hybridizing standard or control polynucleotide molecules to the microarray, wherein the standard or control molecules are differentially labeled from the target molecules; and (3) determining the difference in transcript levels, or lack thereof, between the target and standard or control, wherein the difference, or lack thereof, determines the individual's tumor-related status. In a more specific embodiment, the standard or control molecules comprise biomarker-derived polynucleotides from a pool of samples from normal individuals, a pool of samples from normal adjacent tissue, or a pool of tumor samples from individuals with cancer. In a preferred embodiment, the standard or control is artificially-generated pool of biomarker-derived polynucleotides, which pool is designed to mimic the level of biomarker expression exhibited by clinical samples of normal or cancer tumor tissue having a particular clinical indication (i.e. cancerous or non-cancerous; Wnt/β-catenin pathway regulated or deregulated). In another specific embodiment, the control molecules comprise a pool derived from normal or cancer cell lines.

The present invention provides a set of biomarkers useful for distinguishing deregulated from regulated Wnt/β-catenin pathway tumor types. Thus, in one embodiment of the above method, the level of polynucleotides (i.e., mRNA or polynucleotides derived therefrom) in a sample from an individual, expressed from the biomarkers provided in Table 3 are compared to the level of expression of the same biomarkers from a control, wherein the control comprises biomarker-related polynucleotides derived from deregulated Wnt/β-catenin signaling pathway tumor samples, regulated Wnt/β-catenin signaling pathway tumor samples, or both. The comparison may be to both deregulated and regulated Wnt/β-catenin signaling pathway tumor samples, and the comparison may be to polynucleotide pools from a number of deregulated and regulated Wnt/β-catenin signaling pathway tumor samples, respectively. Where the individual's biomarker expression most closely resembles or correlates with the deregulated control, and does not resemble or correlate with the regulated control, the individual is classified as having a deregulated Wnt/β-catenin signaling pathway. Where the pool is not pure deregulated or regulated Wnt/β-catenin signaling pathway type tumors samples, for example, a sporadic pool is used, a set of experiments using individuals with known Wnt/β-catenin signaling pathway status may be hybridized against the pool in order to define the expression templates for the deregulated and regulated group. Each individual with unknown Wnt/β-catenin signaling pathway status is hybridized against the same pool and the expression profile is compared to the template(s) to determine the individual's Wnt/β-catenin signaling pathway status.

In another specific embodiment, the method comprises:

(i) calculating a measure of similarity between a first expression profile and a deregulated Wnt/β-catenin signaling pathway template, or calculating a first measure of similarity between said first expression profile and said deregulated Wnt/β-catenin signaling pathway template and a second measure of similarity between said first expression profile and a regulated Wnt/β-catenin signaling pathway template, said first expression profile comprising the expression levels of a first plurality of genes in the cell sample, said deregulated Wnt/β-catenin signaling pathway template comprising expression levels of said first plurality of genes that are average expression levels of the respective genes in a plurality of cell samples having at least one or more components of said Wnt/β-catenin signaling pathway with abnormal activity, and said regulated Wnt/β-catenin signaling pathway template comprising expression levels of said first plurality of genes that are average expression levels of the respective genes in a plurality of cell samples not having at least one or more components of said Wnt/β-catenin signaling pathway with abnormal activity, said first plurality of genes consisting of at least 5 of the genes for which biomarkers are listed in Table 3;

(ii) classifying said cell sample as having said deregulated Wnt/β-catenin signaling pathway if said first expression profile has a high similarity to said deregulated Wnt/β-catenin signaling pathway template or has a higher similarity to said deregulated Wnt/β-catenin signaling pathway template than to said regulated Wnt/β-catenin signaling pathway template, or classifying said cell sample as having said regulated Wnt/β-catenin signaling pathway if said first expression profile has a low similarity to said deregulated Wnt/β-catenin signaling pathway template or has a higher similarity to said regulated Wnt/β-catenin signaling pathway template than to said deregulated Wnt/β-catenin signaling pathway template; wherein said first expression profile has a high similarity to said deregulated Wnt/β-catenin signaling pathway template if the similarity to said deregulated Wnt/β-catenin signaling pathway template is above a predetermined threshold, or has a low similarity to said deregulated Wnt/β-catenin signaling pathway template if the similarity to said deregulated Wnt/β-catenin signaling pathway template is below said predetermined threshold; and

(iii) displaying; or outputting to a user interface device, a computer readable storage medium, or a local or remote computer system; the classification produced by said classifying step (ii).

In another specific embodiment, the set of biomarkers may be used to classify a sample from a subject by the Wnt/β-catenin signaling pathway regulation status. The sample may or may not be derived from a tumor. Thus, in one embodiment of the above method, the level of polynucleotides (i.e., mRNA or polynucleotides derived therefrom) in a sample from an individual, expressed from the biomarkers provided in Table 3 are compared to the level of expression of the same biomarkers from a control, wherein the control comprises biomarker-related polynucleotides derived from deregulated Wnt/β-catenin signaling pathway samples, regulated Wnt/β-catenin signaling pathway samples, or both. The comparison may be to both deregulated and regulated Wnt/β-catenin signaling pathway samples, and the comparison may be to polynucleotide pools from a number of deregulated and regulated Wnt/β-catenin signaling pathway samples, respectively. The comparison may also be made to a mixed pool of samples with deregulated and regulated Wnt/β-catenin signaling pathway or unknown samples.

For the above embodiments, the fullest of biomarkers may be used (i.e., the complete set of biomarkers from Table 3). In other embodiments, subsets of the 38 biomarkers may be used or subsets of the “up” (Table 4a) and “down” (Table 4b) arms of the biomarkers may be used.

In another embodiment, the expression profile is a differential expression profile comprising differential measurements of said plurality of genes in a sample derived from a subject versus measurements of said plurality of genes in a control sample. The differential measurements can be xdev, log(ratio), error-weighted log(ratio), or a mean subtracted log(intensity) (see, e.g., PCT publication WO00/39339, published on Jul. 6, 2000; PCT publication WO2004/065545, published Aug. 5, 2004, each of which is incorporated herein by reference in its entirety).

The similarity between the biomarker expression profile of a sample or an individual and that of a control can be assessed a number of ways using any method known in the art. For example, Dai et al. describe a number of different ways of calculating gene expression templates and corresponding biomarker genets useful in classifying breast cancer patients (U.S. Pat. No. 7,171,311; WO2002/103320; WO2005/086891; WO2006015312; WO2006/084272). Similarly, Linsley et al. (US2003/0104426) and Radish et al. (US20070154931) disclose gene biomarker genesets and methods of calculating gene expression templates useful in classifying chronic myelogenous leukemia patients. In the simplest case, the profiles can be compared visually in a printout of expression difference data. Alternatively, the similarity can be calculated mathematically.

In one embodiment, the similarity measure between two patients (or samples) x and y, or patient (or sample) x and a template y, can be calculated using the following equation:

$$S = 1 - [ \begin{matrix}{\sum }_{i = 1}^{{N}_{v}} \frac{( {x}_{i} - \overline{x} )}{{σ}_{{x}_{i}}} \frac{( {y}_{i} - \overline{y} )}{{σ}_{{y}_{i}}} / \\ \sqrt{{\sum }_{i = 1}^{{N}_{v}} {( \frac{( {x}_{i} - \overline{x} )}{{σ}_{{x}_{i}}} )}^{2} {\sum }_{i = 1}^{{N}_{v}} {( \frac{( {y}_{i} - \overline{y} )}{{σ}_{{y}_{i}}} )}^{2}}\end{matrix} ] \qquad ( 4 )$$

In this equation, χ and y are two patients with components of log ratio xᵢ and yᵢ, i=1, 2, . . . , N=4,986. Associated with every value xᵢ is error σₓ_{ᵢ}. The smaller the value σₓ_{ᵢ}, the more reliable the measurement xᵢ.

$$\overline{x} = {\sum }_{i = 1}^{{N}_{v}} \frac{{x}_{i}}{{σ}_{{x}_{i}}^{2}} / {\sum }_{i = 1}^{{N}_{v}} \frac{1}{{σ}_{{x}_{i}}^{2}}$$

is the error-weighted arithmetic mean.

In one embodiment, the similarity is represented by a correlation coefficient between the patient or sample profile and the template. In one embodiment, a correlation coefficient above a correlation threshold indicates high similarity, whereas a correlation coefficient below the threshold indicates low similarity. In some embodiments, the correlation threshold is set as 0.3, 0.4, 0.5, or 0.6. In another embodiment, similarity between a sample or patient profile and a template is represented by a distance between the sample profile and the template. In one embodiment, a distance below a given value indicates a high similarity, whereas a distance equal to or greater than the given value indicates low similarity.

In a preferred embodiment, templates are developed for sample comparison. The template may be defined as the error-weighted log ratio average of the expression difference for the group of biomarker genes able to differentiate the particular Wnt/β-catenin signaling pathway regulation status. For example, templates are defined for deregulated Wnt/β-catenin signaling pathway samples and for regulated Wnt/β-catenin signaling pathway samples. Next, a classifier parameter is calculated. This parameter may be calculated using either expression level differences between the sample and template, or by calculation of a correlation coefficient. Such a coefficient, Pᵢ, can be calculated using the following equation:

Pᵢ=(\vec{c}ᵢ·\vec{y})/(∥\vec{c}ᵢ∥·∥\vec{y}∥) (5)

where i=1 and 2.

As an illustration, in one embodiment, a template for a sample classification based upon one phenotypic endpoint, for example, Wnt/β-catenin signaling pathway deregulated status, is defined as \vec{c}₁ (e.g., a profile consisting of correlation values, C₁, associated with, for example, Wnt/β-catenin signaling pathway regulation status) and/or a template for second phenotypic endpoint, i.e., Wnt/β-catenin signaling pathway regulated status, is defined as \vec{c}₂ (e.g., a profile consisting of correlation values, C₂, associated with, for example, Wnt/β-catenin signaling pathway regulation status). Either one or both of the two classifier parameters (P₁ and P₂) can then be used to measure degrees of similarities between a sample's profile and the templates: P₁ measures the similarity between the sample's profile \vec{y} and the first expression template \vec{c}₁, and P₂ measures the similarity between \vec{y} and the second expression template \vec{c}₂.

Thus, in one embodiment, \vec{y} is classified, for example, as a deregulated Wnt/β-catenin signaling pathway profile if P₁ is greater than a selected correlation threshold or if P₂ is equal to or less than a selected correlation threshold. In another embodiment, \vec{y} is classified, for example, as a regulated Wnt/β-catenin signaling pathway profile if P₁ is less than a selected correlation threshold or if P₂ is above a selected correlation threshold. In still another embodiment, \vec{y} is classified, for example, as a deregulated Wnt/β-catenin signaling pathway profile if P₁ is greater than a first selected correlation threshold and \vec{y} is classified, for example, as a regulated Wnt/β-catenin signaling pathway profile if P₂ is greater than a second selected correlation threshold.

Thus, in a more specific embodiment, the above method of determining a particular tumor-related status of an individual comprises the steps of (1) hybridizing labeled target polynucleotides from an individual to a microarray containing one of the above marker sets; (2) hybridizing standard or control polynucleotides molecules to the microarray, wherein the standard or control molecules are differentially labeled from the target molecules; and (3) determining the ratio (or difference) of transcript levels between two channels (individual and control), or simply the transcript levels of the individual; and (4) comparing the results from (3) to the predefined templates, wherein said determining is accomplished by any means known in the art (see Section 3.4.6 on Methods for Classification of Expression Profiles), and wherein the difference, or lack thereof, determines the individual's tumor-related status.

The method can use the complete set of biomarkers listed in Table 3. However, subsets of the 38 biomarkers, or the “up” (Table 4a) or “down” (Table 4b) arms of the biomarkers may also be used.

In another embodiment, the above method of determining the Wnt/β-catenin pathway regulation status of an individual uses the two “arms” of the 38 biomarkers. The “up” arm comprises the 17 genes whose expression goes up with Wnt/β-catenin pathway activation (see Table 4a), and the “down” arm comprises the 21 genes whose expression goes down with Wnt/β-catenin pathway activation (see Table 4b). When comparing an individual sample with a standard or control, the expression value of gene X in the sample is compared to the expression value of gene X in the standard or control. For each gene in the set of biomarkers, log(10) ratio is created for the expression value in the individual sample relative to the standard or control (differential expression value). A signature “score” may be calculated by determining the mean log(10) ratio of the genes in the “up” and then subtracting the mean log(10) ratio of the genes in the “down” arm. To determine if this signature score is significant, an ANOVA calculation is performed (for example, a two tailed t-test, Wilcoxon rank-sum test, Kolmogorov-Smirnov test, etc.), in which the expression values of the genes in the two opposing arms are compared to one another. For example, if the two tailed t-test is used to determine whether the mean log(10) ratio of the genes in the “up” arm is significantly different than the mean log(10) ratio of the genes in the “down” arm, a p-value of <0.05 indicates that the signature in the individual sample is significantly different from the standard or control. If the signature score for a sample is above a pre-determined threshold, then the sample is considered to have deregulation of the Wnt/β-catenin signaling pathway. The pre-determined threshold may be 0, or may be the mean, median, or a percentile of signature scores of a collection of samples or a pooled sample used as a standard or control. In an alternative embodiment, a subset of at least 3, 5, 10, and 15 of the 17 “up” genes from Table 4a and a subset of at least 3, 5, 10, and 15 of the 21 “down” genes from Table 4b may be used for calculating this signature score. It will be recognized by those skilled in the art that other differential expression values, besides log(10) ratio may be used for calculating a signature score, as long as the value represents an objective measurement of transcript abundance of the biomarker gene. Examples include, but are not limited to: xdev, error-weighted log (ratio), and mean subtracted log(intensity).

In yet another embodiment, the signature score of a sample is defined as the average expression level (such as mean log(ratio)) of the complete set of 38 biomarkers or a subset of these biomarkers, regardless of “arm.” If the signature score for a sample is above a pre-determined threshold, then the sample is considered to have deregulation of the Wnt/β-catenin signaling pathway. The pre-determined threshold may be 0, or may be the mean, median, or a percentile of signature scores of a collection of samples or a pooled sample used as a standard or control.

The use of the biomarkers is not limited to distinguishing or classifying particular tumor types, such as colon cancer, as having deregulated or regulated Wnt/β-catenin signaling pathway. The biomarkers may be used to classify cell samples from any cancer type, where aberrant Wnt/β-catenin signaling may be implicated. Aberrant Wnt/β-catenin pathway signaling has been discovered in a wide variety of cancers, including melanoma, hepatocellular carconima, osteosarcoma, and many tumors (uterine, ovarian, lung, gastric, and renal) (Luu et al., 2004, Curr. Cancer Drug Targets 4:653-671; Reya and Clevers, 2005, Nature 434:843-850; Moon et al., 2004, Nat. Rev. Genet. 5:691-701).

The use of the biomarkers is also not restricted to distinguishing or classifying cell samples as having deregulated or regulated Wnt/β-catenin signaling pathway for cancer-related conditions, and may be applied in a variety of phenotypes or conditions, in which aberrant Wnt/β-catenin signaling plays a role, or the level of Wnt/β-catenin signaling activity is sought. For example, the biomarkers may be useful for classifying cell samples for bone and joint disorders, such as, but not limited to, osteoporosis, rheumatoid arthritis, sclerosteosis, van Buchem syndrome, osteoporosis pseudoglioma syndrome. The Wnt/β-catenin signaling pathway has previously been implicated in bone and joint formation and regeneration (Boyden et al, 2002, N. Engl. J. Med. 346:1513-1521; Gong et al., 2001, Cell 107:513-523; Little et al., 2002, Am. J. Hum. Genet. 70:11-19; Diana et al., 2007, Nat. Med. 13:156-163; Baron and Rawadi, 2007, Endocrin. 148:2635-2643; Kim et al., 2007, J. Bone Mineral Res_{—} 22:1913-1923). Wnt/β-catenin signaling has also been implicated in the development of diabetes (Jin, 2008, Diabetologia, e-publication ahead of print, Aug. 12, 2008); retinal development and disease (Lad et al., 2008, Stem Cells Dev. E-publication ahead of print Aug. 8, 2008); neurodegernative disorders (Caraci et al, 2008, Neurochem. Res., E-publication ahead of print, Apr. 22, 2008).

### 3.4.2 Methods of Predicting Response to Treatment and Assigning Treatment

The invention provides a set of biomarkers useful for distinguishing samples from those patients who are predicted to respond to treatment with an agent that modulates the Wnt/β-catenin signaling pathway from patients who are not predicted to respond to treatment an agent that modulates the Wnt/β-catenin signaling pathway. Thus, the invention further provides a method for using these biomarkers for determining whether an individual with cancer is a predicted responder to treatment with an agent that modulates the Wnt/β-catenin signaling pathway. In one embodiment, the invention provides for a method of predicting response of a cancer patient to an agent that modulates the Wnt/β-catenin signaling pathway comprising (1) comparing the level of expression of the biomarkers listed in Table 3 in a sample taken from the individual to the level of expression of the same biomarkers in a standard or control, where the standard or control levels represent those found in a sample having a deregulated Wnt/β-catenin signaling; and (2) determining whether the level of the biomarker-related polynucleotides in the sample from the individual is significantly different than that of the control, wherein if no substantial difference is found, the patient is predicted to respond to treatment with an agent that modulates the Wnt/β-catenin signaling pathway, and if a substantial difference is found, the patient is predicted not to respond to treatment with an agent that modulates the Wnt/β-catenin signaling pathway. Persons of skill in the art will readily see that the standard or control levels may be from a sample having a regulated Wnt/β-catenin signaling pathway. In a more specific embodiment, both controls are run. In case the pool is not pure “Wnt/β-catenin regulated” or “Wnt/β-catenin deregulated,” a set of experiments of individuals with known responder status may be hybridized against the pool to define the expression templates for the predicted responder and predicted non-responder group. Each individual with unknown outcome is hybridized against the same pool and the resulting expression profile is compared to the templates to predict its outcome.

Wnt/β-catenin signaling pathway deregulation status of a tumor may indicate a subject that is responsive to treatment with an agent that modulates the Wnt/β-catenin signaling pathway. Therefore, the invention provides for a method of determining or assigning a course of treatment of a cancer patient, comprising determining whether the level of expression of the 38 biomarkers of Table 3, or a subset thereof, correlates with the level of these biomarkers in a sample representing deregulated Wnt/β-catenin signaling pathway status or regulated Wnt/β-catenin signaling pathway status; and determining or assigning a course of treatment, wherein if the expression correlates with the deregulated Wnt/β-catenin signaling pathway status pattern, the tumor is treated with an agent that modulates the Wnt/β-catenin signaling pathway.

As with the diagnostic biomarkers, the method can use the complete set of biomarkers listed in Table 3. However, subsets of the 38 biomarkers may also be used. In another embodiment, a subset of at least 5, 10, 15, 20, 25, 30, and 35 biomarkers drawn from the set of 38, can be used to predict the response of a subject to an agent that modulates the Wnt/β-catenin signaling pathway or assign treatment to a subject.

Classification of a sample as “predicted responder” or “predicted non-responder” is accomplished substantially as for the diagnostic biomarkers described above, wherein a template is generated to which the biomarker expression levels in the sample are compared.

In another embodiment, the above method of using Wnt/β-catenin pathway regulation status of an individual to predict treatment response or assign treatment uses the two “arms” of the 39 biomarkers. The “up” arm comprises the genes whose expression goes up with Wnt/β-catenin pathway activation (see Table 4a), and the “down” arm comprises the genes whose expression goes down with Wnt/β-catenin pathway activation (see Table 4b). When comparing an individual sample with a standard or control, the expression value of gene X in the sample is compared to the expression value of gene X in the standard or control. For each gene in the set of biomarkers, log(10) ratio is created for the expression value in the individual sample relative to the standard or control. A signature “score” may be calculated by determining the mean log(10) ratio of the genes in the “up” and then subtracting the mean log(10) ratio of the genes in the “down” arm. If the signature score is above a pre-determined threshold, then the sample is considered to have deregulation of the Wnt/β-catenin signaling pathway. The pre-determined threshold may be 0, or may be the mean, median, or a percentile of signature scores of a collection of samples or a pooled sample used as a standard of control. To determine if this signature score is significant, an ANOVA calculation is perfoiiued (for example, a two tailed t-test, Wilcoxon rank-sum test, Kolmogorov-Smirnov test, etc.), in which the expression values of the genes in the two opposing arms are compared to one another. For example, if the two tailed t-test is used to determine whether the mean log(10) ratio of the genes in the “up” arm is significantly different than the mean log(10) ratio of the genes in the “down” arm, a p-value of <0.05 indicates that the signature in the individual sample is significantly different from the standard or control. In an alternative embodiment, a subset of at least 3, 5, 10, and 15 of the 17 “up” genes from Table 4a and a subset of at least 3, 5, 10, and 15 of the 21 “down” genes from Table 4b may be used for calculating this signature score. In yet another embodiment, the signature score of a sample is defined as the average expression level of the complete set of 38 biomarkers or a subset of these biomarkers, regardless of “arm.” It will be recognized by those skilled in the art that other differential expression values, besides log(10) ratio may be used for calculating a signature score, as long as the value represents an objective measurement of transcript abundance of the biomarker gene. Examples include, but are not limited to: xdev, error-weighted log(ratio), and mean subtracted log(intensity).

In yet another embodiment, the signature score of a sample is defined as the average expression level (such as mean log(ratio)) of the complete set of 38 biomarkers or a subset of these biomarkers, regardless of “arm.” If the signature score for a sample is above a pre-determined threshold, then the sample is considered to have deregulation of the Wnt/β-catenin signaling pathway. The pre-determined threshold may be 0, or may be the mean, median, or a percentile of signature scores of a collection of samples or a pooled sample used as a standard or control.

The use of the biomarkers is not restricted to predicting response to agents that modulate Wnt/β-catenin signaling pathway for cancer-related conditions, and may be applied in a variety of phenotypes or conditions, clinical or experimental, in which gene expression plays a role. Where a set of biomarkers has been identified that corresponds to two or more phenotypes, the biomarker sets can be used to distinguish these phenotypes. For example, the phenotypes may be the diagnosis and/or prognosis of clinical states or phenotypes associated with cancers and other disease conditions, or other physiological conditions, prediction of response to agents that modulate pathways other than the Wnt/β-catenin signaling pathway, wherein the expression level data is derived from a set of genes correlated with the particular physiological or disease condition.

The use of the biomarkers is not limited to predicting response to agents that modulate Wnt/β-catenin signaling pathway for a particular cancer type, such as colon cancer. The biomarkers may be used to predict response to agents in any cancer type, where aberrant Wnt/β-catenin signaling may be implicated. Aberrant Wnt/β-catenin pathway signaling has been discovered in a wide variety of cancers, including melanoma, hepatocellular carconima, osteosarcoma, and many tumors (uterine, ovarian, lung, gastric, and renal) (Luu et al., 2004, Curr. Cancer Drug Targets 4:653-671; Reya and Clevers, 2005, Nature 434:843-850; Moon et al., 2004, Nat. Rev. Genet. 5:691-701).

The use of the biomarkers is also not restricted to predicting response to agents that modulate Wnt/β-catenin signaling pathway for cancer-related conditions, and may be applied in a variety of phenotypes or conditions, in which aberrant Wnt/β-catenin signaling plays a role, or the level of Wnt/β-catenin signaling activity is sought. For example, the biomarkers may be useful for predicting response to agents that modulate the Wnt/β-catenin signaling pathway in subjects with bone or joint disorders, such as, but not limited to, osteoporosis, rheumatoid arthritis, sclerosteosis, van Buchem syndrome, osteoporosis pseudoglioma syndrome. The Wnt/β-catenin signaling pathway has previously been implicated in bone and joint formation and regeneration (Boyden et al, 2002, N. Engl. J. Med. 346:1513-1521; Gong et al., 2001, Cell 107:513-523; Little et al., 2002, Am. J. Hum. Genet. 70:11-19; Diarra et al., 2007, Nat. Med. 13:156-163; Baron and Rawadi, 2007, Endocrin. 148:2635-2643; Kim et al., 2007, J. Bone Mineral Res. 22:1913-1923). Wnt/β-catenin signaling has also been implicated in the development of diabetes (Jin, 2008, Diabetologia, e-publication ahead of print, Aug. 12, 2008); retinal development and disease (Lad et al., 2008, Stem Cells Dev. E-publication ahead of print Aug. 8, 2008); neurodegenerative disorders (Caraci et al, 2008, Neurochem. Res., E-publication ahead of print, Apr. 22, 2008).

### 3.4.3 Method of Determining Whether an Agent Modulates the Wnt/β-catenin Signaling Pathway

The invention provides a set of biomarkers useful for and methods of using the biomarkers for identifying or evaluating an agent that is predicted to modify or modulate the Wnt/β-catenin signaling pathway in a subject. “Wnt/β-catenin signaling pathway” is initiated by binding of the Writ ligands (including, but not limited to Wnt1, Wnt2, Wnt2B/13, Wnt3, Wnt3A, Wnt4, Wnt5A, Wnt5B, Wnt6, Wnt7A, Wnt8A, Wnt8B, Wnt9A, Wnt9B, Wnt10A, Wnt10B, Wnt11, and Wnt16) to the co-receptor Frizzled/LRP5/6 complex. Frizzled interacts with Dishevelled, a cytoplasmic protein that functions upstream of β-catenin and GSK3β, leading to the inactivation of the destruction complex. Upon destruction complex inactivation, stabilized β-catenin is transported to the nucleus where it regulates the activity of TCF/LEF family transcription factors. β-catenin induces expression of a large number of genes, including genes involved in proliferation (c-Myc and Cyclin D1) and feedback regulation of the pathway (Axin-2 and LEF1). In this application, unless otherwise specified, it will be understood that “Wnt/β-catenin signaling pathway” refers to signaling through canonical Wnt/β-catenin signaling pathway, which controls the intracellular level of the proto-oncoprotein β-catenin.

Agents affecting the Wnt/β-catenin signaling pathway include small molecule compounds; proteins or peptides (including antibodies); siRNA, shRNA, or microRNA molecules; or any other agents that modulate one or more genes or proteins that function within the Wnt/β-catenin signaling pathway or other signaling pathways that interact with the Wnt/β-catenin signaling pathway, such as the Notch pathway.

“Wnt/β-catenin pathway agent” refers to an agent which modulates the canonical Wnt/β-catenin pathway signaling. A Wnt/β-catenin pathway inhibitor inhibits the canonical Wnt/β-catenin pathway signaling. Molecular targets of such agents may include β-catenin, TCF4, APC, axin, GSK3β and any of the genes listed in Table 1. Such agents are known in the art and include, but are not limited to: thiazolidinediones (Wang et al., 2008, J. Surg. Res. Jun. 27, 2008 e-publication ahead of print); PKF115-584 (Doghman et al., 2008, J. Clin. Endocrinol. Metab. E-publication ahead of print, doi: 10.1210/jc.2008-0247); bis[2-(acylamino)phenyl]disulfide (Yamakawa et al., 2008, Biol Pharm. Bull. 31:916-920); FH535 (Handeli and Simon, 2008, Mol. Cancer Ther. 7:521-529); suldinac (Han et al., 2008, Fur. J. Pharmacol. 583:26-31); cyclooxygenase-2 inhibitor celecoxib (Tuynman et al., 2008, Cancer Res. 68:1213-1220); reverse-turn mimetic compounds (U.S. Pat. No. 7,232,822); β-catenin inhibitor compound 1 (WO2005021025); fusicoccin analog (WO2007062243); and FZD10 modulators (WO2008061020). The siRNA agents against target genes listed in the Examples that passed the tertiary validation screen are also exemplary Wnt/β-catenin pathway agents (see also Table 5).

In one embodiment, the method for measuring the effect or determining whether an agent modulates the Wnt/β-catenin signaling pathway comprises: (1) comparing the level of expression of the biomarkers listed in Table 3 in a sample treated with an agent to the level of expression of the same biomarkers in a standard or control, wherein the standard or control levels represent those found in a vehicle-treated sample; and (2) determining whether the level of the biomarker-related polynucleotides in the treated sample is significantly different than that of the vehicle-treated control, wherein if no substantial difference is found, the agent is predicted not to have an modulate the Wnt/β-catenin signaling pathway, and if a substantial difference is found, the agent is predicted to modulate the Wnt/β-catenin signaling pathway. In a more specific embodiment, the invention provides a subset of at least 5, 10, 15, 20, 25, 30, and 35 biomarkers, drawn from the set of 38, that can be used to measure or determine the effect of an agent on the Wnt/β-catenin signaling pathway.

In another embodiment, the above method of measuring the effect of an agent on the Wnt/β-catenin signaling pathway uses the two “arms” of the 38 biomarkers. The “up” arm comprises the genes whose expression goes up with Wnt/β-catenin pathway activation (see Table 4a), and the “down” arm comprises the genes whose expression goes down with Wnt/β-catenin pathway activation (see Table 4b). When comparing an individual sample with a standard or control, the expression value of gene X in the sample is compared to the expression value of gene X in the standard or control. For each gene in the set of biomarkers, a log(10) ratio is created for the expression value in the individual sample relative to the standard or control. A signature “score” is calculated by determining the mean log(10) ratio of the genes in the “up” arm and the subtracting the mean log(10) ratio of the genes in the “down” arm. If the signature score is above a pre-determined threshold, then the sample is considered to have deregulation of the Wnt/β-catenin signaling pathway (i.e., the agent modulates the Wnt/β-catenin signaling pathway). The pre-determined threshold may be 0, or may be the mean, median, or a percentile of signature scores of a collection of samples or a pooled sample used as a standard or control. To determine if this signature score is significant, an ANOVA calculation is performed (for example, a two tailed t-test, Wilcoxon rank-sum test, Kolmogorov-Smirnov test, etc.), in which the expression values of the genes in the two opposing arms are compared to one another. For example, if the two tailed t-test is used to determine whether the mean log(10) ratio of the genes in the “up” arm is significantly different than the mean log(10) ratio of the genes in the “down” arm, a p-value of <0.05 indicates that the signature in the individual sample is significantly different from the standard or control. Alternatively, a subset of at least 3, 5, 10, and 15 biomarkers, drawn from the “up” arm (see Table 4a) and a subset of at least 3, 5, 10, and 15 biomarkers from the “down” arm (see Table 4b) may be used for calculating this signature score. It will be recognized by those skilled in the art that other differential expression values, besides log(10) ratio may be used for calculating a signature score, as long as the value represents an objective measurement of transcript abundance of the biomarker gene. Examples include, but are not limited to: xdev, error-weighted log (ratio), and mean subtracted log(intensity).

In yet another embodiment, the signature score of a sample is defined as the average expression level (such as mean log(ratio)) of the complete set of 38 biomarkers or a subset of these biomarkers, regardless of “arm.” If the signature score for a sample is above a pre-determined threshold, then the sample is considered to have deregulation of the Wnt/3-catenin signaling pathway. The pre-determined threshold may be 0, or may be the mean, median, or a percentile of signature scores of a collection of samples or a pooled sample used as a standard or control.

The use of the biomarkers is not restricted to determining whether an agent modulates Wnt/β-catenin signaling pathway for cancer-related conditions, and may be applied in a variety of phenotypes or conditions, clinical or experimental, in which gene expression plays a role. Where a set of biomarkers has been identified that corresponds to two or more phenotypes, the biomarker sets can be used to distinguish these phenotypes. For example, the phenotypes may be the diagnosis and/or prognosis of clinical states or phenotypes associated with cancers and other disease conditions, or other physiological conditions, prediction of response to agents that modulate pathways other than the Wnt/β-catenin signaling pathway, wherein the expression level data is derived from a set of genes correlated with the particular physiological or disease condition.

The use of the biomarkers is not limited to determining whether an agent modulates the Wnt/β-catenin signaling pathway for a particular cancer type, such as colon cancer. The biomarkers may be used to determine whether an agent modulates the Wnt/β-catenin for any cancer type, where aberrant Wnt/β-catenin signaling may be implicated. Aberrant Wnt/β-catenin pathway signaling has been discovered in a wide variety of cancers, including melanoma, hepatocellular carconima, osteosarcoma, and many tumors (uterine, ovarian, lung, gastric, and renal) (Luu et al., 2004, Curr. Cancer Drug Targets 4:653-671; Reya and Clevers, 2005, Nature 434:843-850; Moon et al., 2004, Nat. Rev. Genet. 5:691-701).

The use of the biomarkers is also not restricted determining whether an agent modulates the Wnt/β-catenin signaling pathway for cancer-related conditions, and may be applied for agents for a variety of phenotypes or conditions, in which aberrant Wnt/β-catenin signaling plays a role, or the level of Wnt/β-catenin signaling activity is sought. For example, the biomarkers may be useful for determining whether an agent modulates the Wnt/β-catenin signaling pathway, for treatment of bone or joint disorders, such as, but not limited to, osteoporosis, rheumatoid arthritis, sclerosteosis, van Buchem syndrome, osteoporosis pseudoglioma syndrome. The Wnt/β-catenin signaling pathway has previously been implicated in bone and joint formation and regeneration (Boyden et al, 2002, N. Engl. J. Med. 346:1513-1521; Gong et al., 2001, Cell 107:513-523; Little et al., 2002, Am. J. Hum. Genet. 70:11-19; Diarra et al., 2007, Nat. Med. 13:156-163; Baron and Rawadi, 2007, Endocrin. 148:2635-2643; Kim et al., 2007, J. Bone Mineral Res. 22:1913-1923). Wnt/β-catenin signaling has also been implicated in the development of diabetes (Jin, 2008, Diabetologia, e-publication ahead of print, Aug. 12, 2008); retinal development and disease (Lad et al., 2008, Stem Cells Dev. E-publication ahead of print Aug. 8, 2008); neurodegernative disorders (Caraci et al, 2008, Neurochem. Res., E-publication ahead of print, Apr. 22, 2008).

### 3.4.4 Method of Measuring Pharmacodynamic Effect of an Agent

The invention provides a set of biomarkers useful for measuring the pharmacodynamic effect of an agent on the Wnt/β-catenin signaling pathway. The biomarkers provided may be used to monitor modulation of the Wnt/β-catenin signaling pathway at various time points following treatment with said agent in a patient or sample. Thus, the invention further provides a method for using these biomarkers as an early evaluation for efficacy of an agent which modulates the Wnt/β-catenin signaling pathway. In one embodiment, the invention provides for a method of measuring pharmacodynamic effect of an agent that modulates the Wnt/β-catenin signaling pathway in patient or sample comprising: (1) comparing the level of expression of the biomarkers listed in Table 3 in a sample treated with an agent to the level of expression of the same biomarkers in a standard or control, wherein the standard or control levels represent those found in a vehicle-treated sample; and (2) determining whether the level of the biomarker-related polynucleotides in the treated sample is significantly different than that of the vehicle-treated control, wherein if no substantial difference is found, the agent is predicted not to have an pharmacodynamic effect on the Wnt/β-catenin signaling pathway, and if a substantial difference is found, the agent is predicted to have an pharmacodynamic effect on the Wnt/β-catenin signaling pathway. In a more specific embodiment, the invention provides a subset of at least 5, 10, 15, 20, 25, 30, and 35 biomarkers, drawn from the set of 38 that can be used to monitor pharmacodynamic activity of an agent on the Wnt/β-catenin signaling pathway.

In another embodiment, the above method of measuring pharmacodynamic activity of an agent on the Wnt/β-catenin signaling pathway uses the two “arms” of the 38 biomarkers. The “up” arm comprises the genes whose expression goes up with Wnt/β-catenin pathway activation (see Table 4a), and the “down” arm comprises the genes whose expression goes down with Wnt/β-catenin pathway activation (see Table 4b). When comparing an individual sample with a standard or control, the expression value of gene X in the sample is compared to the expression value of gene X in the standard or control. For each gene in the set of biomarkers, a log(10) ratio is created for the expression value in the individual sample relative to the standard or control. A signature “score” is calculated by determining the mean log(10) ratio of the genes in the “up” arm and the subtracting the mean log(10) ratio of the genes in the “down” arm. If the signature score is above a pre-determined threshold, then the sample is considered to have deregulation of the Wnt/β-catenin signaling pathway. The pre-determined threshold may be 0, or may be the mean, median, or a percentile of signature scores of a collection of samples or a pooled sample used as a standard or control. To determine if this signature score is significant, an ANOVA calculation is performed (for example, a two tailed t-test, Wilcoxon rank-sum test, Kolmogorov-Smirnov test, etc.), in which the expression values of the genes in the two opposing arms are compared to one another. For example, if the two tailed t-test is used to determine whether the mean log(10) ratio of the genes in the “up” arm is significantly different than the mean log(10) ratio of the genes in the “down” arm, a p-value of <0.05 indicates that the signature in the individual sample is significantly different from the standard or control. Alternatively, a subset of at least 3, 5, 10, and 15 biomarkers, drawn from the “up” arm (see Table 4a) and a subset of at least 3, 5, 10, and 15 biomarkers from the “down” arm (see Table 4b) may be used for calculating this signature score. It will be recognized by those skilled in the art that other differential expression values, besides log(10) ratio may be used for calculating a signature score, as long as the value represents an objective measurement of transcript abundance of the biomarker gene. Examples include, but are not limited to: xdev, error-weighted log(ratio), and mean subtracted log(intensity).

In yet another embodiment, the signature score of a sample is defined as the average expression level (such as mean log(ratio)) of the complete set of 38 biomarkers or a subset of these biomarkers, regardless of “arm.” If the signature score for a sample is above a pre-determined threshold, then the sample is considered to have deregulation of the Wnt/β-catenin signaling pathway. The pre-determined threshold may be 0, or may be the mean, median, or a percentile of signature scores of a collection of samples or a pooled sample used as a standard or control.

### 3.4.5 Improving Sensitivity to Expression Level Differences

In using the biomarkers disclosed herein, and, indeed, using any sets of biomarkers to differentiate an individual or subject having one phenotype from another individual or subject having a second phenotype, one can compare the absolute expression of each of the biomarkers in a sample to a control; for example, the control can be the average level of expression of each of the biomarkers, respectively, in a pool of individuals or subjects. To increase the sensitivity of the comparison, however, the expression level values are preferably transformed in a number of ways.

For example, the expression level of each of the biomarkers can be normalized by the average expression level of all markers the expression level of which is determined, or by the average expression level of a set of control genes. Thus, in one embodiment, the biomarkers are represented by probes on a microarray, and the expression level of each of the biomarkers is normalized by the mean or median expression level across all of the genes represented on the microarray, including any non-biomarker genes. In a specific embodiment, the normalization is carried out by dividing the median or mean level of expression of all of the genes on the microarray. In another embodiment, the expression levels of the biomarkers is normalized by the mean or median level of expression of a set of control biomarkers. In a specific embodiment, the control biomarkers comprise a set of housekeeping genes. In another specific embodiment, the normalization is accomplished by dividing by the median or mean expression level of the control genes.

The sensitivity of a biomarker-based assay will also be increased if the expression levels of individual biomarkers are compared to the expression of the same biomarkers in a pool of samples. Preferably, the comparison is to the mean or median expression level of each the biomarker genes in the pool of samples. Such a comparison may be accomplished, for example, by dividing by the mean or median expression level of the pool for each of the biomarkers from the expression level each of the biomarkers in the sample. This has the effect of accentuating the relative differences in expression between biomarkers in the sample and markers in the pool as a whole, making comparisons more sensitive and more likely to produce meaningful results that the use of absolute expression levels alone. The expression level data may be transformed in any convenient way; preferably, the expression level data for all is log transformed before means or medians are taken.

In performing comparisons to a pool, two approaches may be used. First, the expression levels of the markers in the sample may be compared to the expression level of those markers in the pool, where nucleic acid derived from the sample and nucleic acid derived from the pool are hybridized during the course of a single experiment. Such an approach requires that new pool nucleic acid be generated for each comparison or limited numbers of comparisons, and is therefore limited by the amount of nucleic acid available. Alternatively, and preferably, the expression levels in a pool, whether normalized and/or transformed or not, are stored on a computer, or on computer-readable media, to be used in comparisons to the individual expression level data from the sample (i.e., single-channel data).

Thus, the current invention provides the following method of classifying a first cell or organism as having one of at least two different phenotypes, where the different phenotypes comprise a first phenotype and a second phenotype. The level of expression of each of a plurality of genes in a first sample from the first cell or organism is compared to the level of expression of each of said genes, respectively, in a pooled sample from a plurality of cells or organisms, the plurality of cells or organisms comprising different cells or organisms exhibiting said at least two different phenotypes, respectively, to produce a first compared value. The first compared value is then compared to a second compared value, wherein said second compared value is the product of a method comprising comparing the level of expression of each of said genes in a sample from a cell or organism characterized as having said first phenotype to the level of expression of each of said genes, respectively, in the pooled sample. The first compared value is then compared to a third compared value, wherein said third compared value is the product of a method comprising comparing the level of expression of each of the genes in a sample from a cell or organism characterized as having the second phenotype to the level of expression of each of the genes, respectively, in the pooled sample. Optionally, the first compared value can be compared to additional compared values, respectively, where each additional compared value is the product of a method comprising comparing the level of expression of each of said genes in a sample from a cell or organism characterized as having a phenotype different from said first and second phenotypes but included among the at least two different phenotypes, to the level of expression of each of said genes, respectively, in said pooled sample. Finally, a determination is made as to which of said second, third, and, if present, one or more additional compared values, said first compared value is most similar, wherein the first cell or organism is determined to have the phenotype of the cell or organism used to produce said compared value most similar to said first compared value.

In a specific embodiment of this method, the compared values are each ratios of the levels of expression of each of said genes. In another specific embodiment, each of the levels of expression of each of the genes in the pooled sample is normalized prior to any of the comparing steps. In a more specific embodiment, the normalization of the levels of expression is carried out by dividing by the median or mean level of the expression of each of the genes or dividing by the mean or median level of expression of one or more housekeeping genes in the pooled sample from said cell or organism. In another specific embodiment, the normalized levels of expression are subjected to a log transform, and the comparing steps comprise subtracting the log transform from the log of the levels of expression of each of the genes in the sample. In another specific embodiment, the two or more different phenotypes are different regulation status of the Wnt/β-catenin signaling pathway. In still another specific embodiment, the two or more different phenotypes are different predicted responses to treatment with an agent that modulates the Wnt/β-catenin signaling pathway. In yet another specific embodiment, the levels of expression of each of the genes, respectively, in the pooled sample or said levels of expression of each of said genes in a sample from the cell or organism characterized as having the first phenotype, second phenotype, or said phenotype different from said first and second phenotypes, respectively, are stored on a computer or on a computer-readable medium.

In another specific embodiment, the two phenotypes are deregulated or Wnt/β-catenin signaling pathway status. In another specific embodiment, the two phenotypes are predicted Wnt/β-catenin signaling pathway-agent responder status. In yet another specific embodiment, the two phenotypes are pharmacodynamic effect and no pharmcodynamic effect of an agent on the Wnt/β-catenin signaling pathway.

In another specific embodiment, the comparison is made between the expression of each of the genes in the sample and the expression of the same genes in a pool representing only one of two or more phenotypes. In the context of Wnt/β-catenin signaling pathway status-correlated genes, for example, one can compare the expression levels of Wnt/β-catenin signaling pathway regulation status-related genes in a sample to the average level of the expression of the same genes in a “deregulated” pool of samples (as opposed to a pool of samples that include samples from patients having regulated and deregulated Wnt/β-catenin signaling pathway status). Thus, in this method, a sample is classified as having a deregulated Wnt/β-catenin signaling pathway status if the level of expression of prognosis-correlated genes exceeds a chosen coefficient of correlation to the average “deregulated Wnt/β-catenin signaling pathway” expression profile (i.e., the level of expression of Wnt/β-catenin signaling pathway status-correlated genes in a pool of samples from patients having a “deregulated Wnt/β-catenin signaling pathway status.” Patients or subjects whose expression levels correlate more poorly with the “deregulated Wnt/β-catenin signaling pathway” expression profile (i.e., whose correlation coefficient fails to exceed the chosen coefficient) are classified as having a regulated Wnt/β-catenin signaling pathway status.

Of course, single-channel data may also be used without specific comparison to a mathematical sample pool. For example, a sample may be classified as having a first or a second phenotype, wherein the first and second phenotypes are related, by calculating the similarity between the expression of at least 5 markers in the sample, where the markers are correlated with the first or second phenotype, to the expression of the same markers in a first phenotype template and a second phenotype template, by (a) labeling nucleic acids derived from a sample with a fluorophore to obtain a pool of fluorophore-labeled nucleic acids; (b) contacting said fluorophore-labeled nucleic acid with a microarray under conditions such that hybridization can occur, detecting at each of a plurality of discrete loci on the microarray a flourescent emission signal from said fluorophore-labeled nucleic acid that is bound to said microarray under said conditions; and (c) determining the similarity of marker gene expression in the individual sample to the first and second templates, wherein if said expression is more similar to the first template, the sample is classified as having the first phenotype, and if said expression is more similar to the second template, the sample is classified as having the second phenotype.

### 3.4.6 Methods for Classification of Expression Profiles

In preferred embodiments, the methods of the invention use a classifier for predicting Wnt/β-catenin signaling pathway regulation status of a sample, predicting response to agents that modulate the Wnt/β-catenin signaling pathway, assigning treatment to a subject, and/or measuring pharmacodynamic effect of an agent. The classifier can be based on any appropriate pattern recognition method that receives an input comprising a biomarker profile and provides an output comprising data indicating which patient subset the patient belongs. The classifier can be trained with training data from a training population of subjects. Typically, the training data comprise for each of the subjects in the training population a training marker profile comprising measurements of respective gene products of a plurality of genes in a suitable sample taken from the patient and outcome information, i.e., deregulated or regulated Wnt/β-catenin signaling pathway status.

In preferred embodiments, the classifier can be based on a classification (pattern recognition) method described below, e.g., profile similarity; artificial neural network); support vector machine (SVM); logic regression, linear or quadratic discriminant analysis, decision trees, clustering, principal component analysis, nearest neighbor classifier analysis (described infra). Such classifiers can be trained with the training population using methods described in the relevant sections, infra.

The biomarker profile can be obtained by measuring the plurality of gene products in a cell sample from the subject using a method known in the art, e.g., a method described infra.

Various known statistical pattern recognition methods can be used in conjunction with the present invention. A classifier based on any of such methods can be constructed using the biomarker profiles and Wnt/β-catenin pathway signalling status data of training patients. Such a classifier can then be used to evaluate the Wnt/β-catenin pathway signalling status of a patient based on the patient's biomarker profile. The methods can also be used to identify biomarkers that discriminate between different Wnt/β-catenin signalling pathway regulation status using a biomarker profile and Wnt/β-catenin signalling pathway regulation data of training patients.

A. Profile Matching

A subject can be classified by comparing a biomarker profile obtained in a suitable sample from the subject with a biomarker profile that is representative of a particular phenotypic state. Such a marker profile is also termed a “template profile” or a “template.” The degree of similarity to such a template profile provides an evaluation of the subject's phenotype. If the degree of similarity of the subject marker profile and a template profile is above a predetermined threshold, the subject is assigned the classification represented by the template. For example, a subject's outcome prediction can be evaluated by comparing a biomarker profile of the subject to a predetermined template profile corresponding to a given phenotype or outcome, e.g., a Wnt/β-catenin signalling pathway template comprising measurements of the plurality of biomarkers which are representative of levels of the biomarkers in a plurality of subjects that have tumors with deregulated Wnt/β-catenin signalling pathway status.

In one embodiment, the similarity is represented by a correlation coefficient between the subject's profile and the template. In one embodiment, a correlation coefficient above a correlation threshold indicates a high similarity, whereas a correlation coefficient below the threshold indicates a low similarity.

In a specific embodiment, Pᵢ measures the similarity between the subject's profile \vec{y} and a template profile comprising measurements of marker gene products representative of measurements of marker gene products in subjects having a particular outcome or phenotype, e.g., deregulated Wnt/β-catenin signalling pathway status \vec{z}₁ or a regulated Wnt/β-catenin signalling pathway status \vec{z}₂. Such a coefficient, Pᵢ, can be calculated using the following equation:

Pᵢ=(\vec{z}ᵢ·\vec{y})/(∥\vec{z}ᵢ∥·∥\vec{y}∥)

where i designates the ith template. Thus, in one embodiment, \vec{y} is classified as a deregulated Wnt/β-catenin signalling pathway profile if P₁ is greater than a selected correlation threshold. In another embodiment, \vec{y} is classified as a regulated Wnt/β-catenin signalling pathway profile if P₂ is greater than a selected correlation threshold. In preferred embodiments, the correlation threshold is set as 0.3, 0.4, 0.5 or 0.6. In another embodiment, \vec{y} is classified as a deregulated Wnt/β-catenin signalling pathway profile if P₁ is greater than P₂, whereas \vec{y} is classified as a regulated Wnt/β-catenin signalling pathway profile if P₁ is less than P₂.

In another embodiment, the correlation coefficient is a weighted dot product of the patient's profile \vec{y} and a template profile, in which measurements of each different marker is assigned a weight.

In another embodiment, similarity between a patient's profile and a template is represented by a distance between the patient's profile and the template. In one embodiment, a distance below a given value indicates high similarity, whereas a distance equal to or greater than the given value indicates low similarity.

In one embodiment, the Euclidian distance according to the formula

Dᵢ=∥\vec{y}−\vec{z}ᵢ∥

is used, where Dᵢ measures the distance between the subject's profile \vec{y} and a template profile comprising measurements of marker gene products representative of measurements of marker gene products in subjects having a particular Wnt/β-catenin signaling pathway regulation status, e.g., the deregulated Wnt/β-catenin signaling pathway \vec{z}₁ or the regulated Wnt/β-catenin signaling pathway template \vec{z}₂. In other embodiments, the Euclidian distance is squared to place progressively greater weight on cellular constituents that are further apart. In alternative embodiments, the distance measure Dᵢ is the Manhattan distance provide by

$${D}_{i} = {\sum }_{n} \vert y ( n ) - {z}_{i} ( n ) \vert$$

where y(n) and zᵢ(n) are respectively measurements of the nth marker gene product in the subject's profile \vec{y} and a template profile.

In another embodiment, the distance is defined as Dᵢ=1−Pᵢ, where Pᵢ is the correlation coefficient or normalized dot product as described above.

In still other embodiments, the distance measure may be the Chebychev distance, the power distance, and percent disagreement, all of which are well known in the art.

B. Artificial Neural Network

In some embodiments, a neural network is used. A neural network can be constructed for a selected set of molecular markers of the invention. A neural network is a two-stage regression or classification model. A neural network has a layered structure that includes a layer of input units (and the bias) connected by a layer of weights to a layer of output units. For regression, the layer of output units typically includes just one output unit. However, neural networks can handle multiple quantitative responses in a seamless fashion.

In multilayer neural networks, there are input units (input layer), hidden units (hidden layer), and output units (output layer). There is, furthermore, a single bias unit that is connected to each unit other than the input units. Neural networks are described in Duda et al., 2001, Pattern Classification, Second Edition, John Wiley & Sons, Inc., New York; and Hastie et al., 2001, The Elements of Statistical Learning, Springer-Verlag, New York.

The basic approach to the use of neural networks is to start with an untrained network, present a training pattern, e.g., biomarker profiles from training patients, to the input layer, and to pass signals through the net and determine the output, e.g., the Wnt/β-catenin signaling pathway regulation status in the training patients, at the output layer. These outputs are then compared to the target values; any difference corresponds to an error. This error or criterion function is some scalar function of the weights and is minimized when the network outputs match the desired outputs. Thus, the weights are adjusted to reduce this measure of error. For regression, this error can be sum-of-squared errors. For classification, this error can be either squared error or cross-entropy (deviation). See, e.g., Hastie et al., 2001, The Elements of Statistical Learning, Springer-Verlag, New York.

Three commonly used training protocols are stochastic, batch, and on-line. In stochastic training, patterns are chosen randomly from the training set and the network weights are updated for each pattern presentation. Multilayer nonlinear networks trained by gradient descent methods such as stochastic back-propagation perform a maximum-likelihood estimation of the weight values in the model defined by the network topology. In batch training, all patterns are presented to the network before learning takes place. Typically, in batch training, several passes are made through the training data. In online training, each pattern is presented once and only once to the net.

In some embodiments, consideration is given to starting values for weights. If the weights are near zero, then the operative part of the sigmoid commonly used in the hidden layer of a neural network (see, e.g., Hastie et al., 2001, The Elements of Statistical Learning, Springer-Verlag, New York) is roughly linear, and hence the neural network collapses into an approximately linear model. In some embodiments, starting values for weights are chosen to be random values near zero. Hence the model starts out nearly linear, and becomes nonlinear as the weights increase. Individual units localize to directions and introduce nonlinearities where needed. Use of exact zero weights leads to zero derivatives and perfect symmetry, and the algorithm never moves. Alternatively, starting with large weights often leads to poor solutions.

Since the scaling of inputs determines the effective scaling of weights in the bottom layer, it can have a large effect on the quality of the final solution. Thus, in some embodiments, at the outset all expression values are standardized to have mean zero and a standard deviation of one. This ensures all inputs are treated equally in the regularization process, and allows one to choose a meaningful range for the random starting weights. With standardization inputs, it is typical to take random uniform weights over the range [−0.7, +0.7].

A recurrent problem in the use of networks having a hidden layer is the optimal number of hidden units to use in the network. The number of inputs and outputs of a network are determined by the problem to be solved. In the present invention, the number of inputs for a given neural network can be the number of molecular markers in the selected set of molecular markers of the invention. The number of output for the neural network will typically be just one. However, in some embodiment more than one output is used so that more than just two states can be defined by the network. If too many hidden units are used in a neural network, the network will have too many degrees of freedom and is trained too long, there is a danger that the network will overfit the data. If there are too few hidden units, the training set cannot be learned. Generally speaking, however, it is better to have too many hidden units than too few. With too few hidden units, the model might not have enough flexibility to capture the nonlinearities in the data; with too many hidden units, the extra weight can be shrunk towards zero if appropriate regularization or pruning, as described below, is used. In typical embodiments, the number of hidden units is somewhere in the range of 5 to 100, with the number increasing with the number of inputs and number of training cases.

One general approach to determining the number of hidden units to use is to apply a regularization approach. In the regularization approach, a new criterion function is constructed that depends not only on the classical training error, but also on classifier complexity. Specifically, the new criterion function penalizes highly complex models; searching for the minimum in this criterion is to balance error on the training set with error on the training set plus a regularization term, which expresses constraints or desirable properties of solutions:

J=Jₚₐₜ+λJ_{reg}.

The parameter λ is adjusted to impose the regularization more or less strongly. In other words, larger values for λ will tend to shrink weights towards zero: typically cross-validation with a validation set is used to estimate λ. This validation set can be obtained by setting aside a random subset of the training population. Other forms of penalty can also be used, for example the weight elimination penalty (see, e.g., Hastie et al., 2001, The Elements of Statistical Learning, Springer-Verlag, New York).

Another approach to determine the number of hidden units to use is to eliminate-prune-weights that are least needed. In one approach, the weights with the smallest magnitude are eliminated (set to zero). Such magnitude-based pruning can work, but is nonoptimal; sometimes weights with small magnitudes are important for learning and training data. In some embodiments, rather than using a magnitude-based pruning approach, Wald statistics are computed. The fundamental idea in Wald Statistics is that they can be used to estimate the importance of a hidden unit (weight) in a model. Then, hidden units having the least importance are eliminated (by setting their input and output weights to zero). Two algorithms in this regard are the Optimal Brain Damage (OBD) and the Optimal Brain Surgeon (OBS) algorithms that use second-order approximation to predict how the training error depends upon a weight, and eliminate the weight that leads to the smallest increase in training error.

Optimal Brain Damage and Optimal Brain Surgeon share the same basic approach of training a network to local minimum error at weight w, and then pruning a weight that leads to the smallest increase in the training error. The predicted functional increase in the error for a change in full weight vector δw is:

$$δ J = {( \frac{\partial J}{\partial w} )}^{t} \cdot δ w + \frac{1}{2} δ {w}^{t} \cdot \frac{{\partial }^{2} J}{\partial {w}^{2}} \cdot δ w + O ( {‖ δ w ‖}^{3} )$$

where

$$\frac{{\partial }^{2} J}{\partial {w}^{2}}$$

is the Hessian matrix. The first term vanishes because we are at a local minimum in error; third and higher order terms are ignored. The general solution for minimizing this function given the constraint of deleting one weight is:

$$δ w = - \frac{{w}_{q}}{{[ {H}^{- 1} ]}_{qq}} {H}^{- 1} \cdot {u}_{q} and {L}_{q} = \frac{1}{2} - \frac{{w}_{q}^{2}}{{[ {H}^{- 1} ]}_{qq}}$$

Here, u_{q} is the unit vector along the qth direction in weight space and L_{q} is approximation to the saliency of the weight q—the increase in training error if weight q is pruned and the other weights updated δw. These equations require the inverse of H. One method to calculate this inverse matrix is to start with a small value, H₀⁻¹=α⁻¹I, where α is a small parameter—effectively a weight constant. Next the matrix is updated with each pattern according to

$${H}_{m + 1}^{- 1} = {H}_{m}^{- 1} - \frac{{H}_{m}^{- 1} {X}_{m + 1} {X}_{m + 1}^{T} {H}_{m}^{- 1}}{\frac{n}{{a}_{m}} + {X}_{m + 1}^{T} {H}_{m}^{- 1} {X}_{M + 1}}$$

where the subscripts correspond to the pattern being presented and αₘ decreases with m. After the full training set has been presented, the inverse Hessian matrix is given by H⁻¹=Hₙ⁻¹. In algorithmic form, the Optimal Brain Surgeon method is:

begin initialize n_{H}, w, θ

train a reasonably large network to minimum error
do compute H⁻¹ by Eqn. 1

${q}^{*} ← arg {min}_{q} {w}_{q}^{2} / ( {2 [ {H}^{- 1} ]}_{qq} ) ( saliency {L}_{q} )$
$w ← w - \frac{{w}_{{q}^{*}}}{{[ {H}^{- 1} ]}_{{q}^{*} {q}^{*}}} {H}^{- 1} {e}_{{q}^{* }} ( saliency {L}_{q} )$

until J(w) > θ

return w

end

The Optimal Brain Damage method is computationally simpler because the calculation of the inverse Hessian matrix in line 3 is particularly simple for a diagonal matrix. The above algorithm terminates when the error is greater than a criterion initialized to be θ. Another approach is to change line 6 to terminate when the change in J(w) due to elimination of a weight is greater than some criterion value.

In some embodiments, a back-propagation neural network (see, for example Abdi, 1994, “A neural network primer”, J. Biol System. 2, 247-283) containing a single hidden layer of ten neurons (ten hidden units) found in EasyNN-Plus version 4.0 g software package (Neural Planner Software Inc.) is used. In a specific example, parameter values within the EasyNN-Plus program are set as follows: a learning rate of 0.05, and a momentum of 0.2. In some embodiments in which the EasyNN-Plus version 4.0 g software package is used, “outlier” samples are identified by performing twenty independently-seeded trials involving 20,000 learning cycles each.

C. Support Vector Machine

In some embodiments of the present invention, support vector machines (SVMs) are used to classify subjects using expression profiles of marker genes described in the present invention. General description of SVM can be found in, for example, Cristianini and Shawe-Taylor, 2000, An Introduction to Support Vector Machines, Cambridge University Press, Cambridge, Baser et al., 1992, “A training algorithm for optimal margin classifiers, in Proceedings of the 5^{th} Annual ACM Workshop on Computational Learning Theory, ACM Press, Pittsburgh, Pa., pp. 142-152; Vapnik, 1998, Statistical Learning Theory, Wiley, New York; Duda, Pattern Classification, Second Edition, 2001, John Wiley & Sons, Inc.; Hastie, 2001, The Elements of Statistical Learning, Springer, N.Y.; and Furey et al, 2000, Bioinformatics 16, 906-914. Applications of SVM in biological applications are described in Jaakkola et al., Proceedings of the 7^{th} International Conference on Intelligent Systems for Molecular Biology, AAAI Press, Menlo Park, Calif. (1999); Brown et al., Proc. Natl. Acad. Sci. 97(1):262-67 (2000); Zien et al., Bioinformatics, 16(9):799-807 (2000); Furey et al., Bioinformatics, 16(10):906-914 (2000)

In one approach, when a SVM is used, the gene expression data is standardized to have mean zero and unit variance and the members of a training population are randomly divided into a training set and a test set. For example, in one embodiment, two thirds of the members of the training population are placed in the training set and one third of the members of the training population are placed in the test set. The expression values for a selected set of genes of the present invention is used to train the SVM. Then the ability for the trained SVM to correctly classify members in the test set is determined. In some embodiments, this computation is performed several times for a given selected set of molecular markers. In each iteration of the computation, the members of the training population are randomly assigned to the training set and the test set. Then, the quality of the combination of molecular markers is taken as the average of each such iteration of the SVM computation.

Support vector machines map a given set of binary labeled training data to a high-dimensional feature space and separate the two classes of data with a maximum margin hyperplane. In general, this hyperplane corresponds to a nonlinear decision boundary in the input space. Let XεR₀ [symbol] be the input vectors, yε{−1,+1} be the labels, and φ: R₀→F be the mapping from input space to feature space. Then the SVM learning algorithm finds a hyperplane (w,b) such that the quantity

$$γ = {min}_{i} {γ}_{i} \{ 〈 w , φ ( {X}_{i} ) 〉 - b \}$$

is maximized, where the vector w has the same dimensionality as F, b is a real number, and γ is called the margin. The corresponding decision function is then

f(X)=sign( [symbol] (w,φ(X) [symbol] −b)

This minimum occurs when

$$w = {\sum }_{i} {α}_{i} {y}_{i} φ ( {X}_{i} )$$

where {αᵢ} are positive real numbers that maximize

$${\sum }_{i} {α}_{i} - {\sum }_{ij} {α}_{i} {α}_{j} {y}_{i} {y}_{j} 〈 φ ( {X}_{i} ) , φ ( {X}_{j} ) 〉$$

$$subject to$$

$${\sum }_{i} {α}_{i} {y}_{i} = 0 , {α}_{i} > 0$$

The decision function can equivalently be expressed as

$$f ( X ) = sign ( {\sum }_{i} {α}_{i} {y}_{i} 〈 φ ( {X}_{i} , φ ( X ) 〉 - b )$$

From this equation it can be seen that the αᵢ associated with the training point Xᵢ expresses the strength with which that point is embedded in the final decision function. A remarkable property of this alternative representation is that only a subset of the points will be associated with a non-zero αᵢ. These points are called support vectors and are the points that lie closest to the separating hyperplane. The sparseness of the a vector has several computational and learning theoretic consequences. It is important to note that neither the learning algorithm nor the decision function needs to represent explicitly the image of points in the feature space, φ(Xᵢ), since both use only the dot products between such images, [symbol] φ(Xᵢ),φ(Xⱼ) [symbol] . Hence, if one were given a function K(X,Y)= [symbol] φ(X),φ(X) [symbol] , one could learn and use the maximum margin hyperplane in the feature space without ever explicitly performing the mapping. For each continuous positive definite function K(X,Y) there exists a mapping φ such that K(X,Y)= [symbol] φ(X),φ(X) [symbol] for all X,YεR₀ (Mercer's Theorem). The function K(X,Y) is called the kernel function. The use of a kernel function allows the support vector machine to operate efficiently in a nonlinear high-dimensional feature spaces without being adversely affected by the dimensionality of that space. Indeed, it is possible to work with feature spaces of infinite dimension. Moreover, Mercer's theorem makes it possible to learn in the feature space without even knowing φ and F. The matrix Kᵢⱼ= [symbol] φ(Xᵢ),φ(Xⱼ) [symbol] is called the kernel matrix. Finally, note that the learning algorithm is a quadratic optimization problem that has only a global optimum. The absence of local minima is a significant difference from standard pattern recognition techniques such as neural networks. For moderate sample sizes, the optimization problem can be solved with simple gradient descent techniques. In the presence of noise, the standard maximum margin algorithm described above can be subject to overfitting, and more sophisticated techniques should be used. This problem arises because the maximum margin algorithm always finds a perfectly consistent hypothesis and does not tolerate training error. Sometimes, however, it is necessary to trade some training accuracy for better predictive power. The need for tolerating training error has led to the development the soft-margin and the margin-distribution classifiers. One of these techniques replaces the kernel matrix in the training phase as follows:

K←K+λI

while still using the standard kernel function in the decision phase. By tuning λ, one can control the training error, and it is possible to prove that the risk of misclassifying unseen points can be decreased with a suitable choice of λ.

If instead of controlling the overall training error one wants to control the trade-off between false positives and false negatives, it is possible to modify K as follows:

K←K+λD

where D is a diagonal matrix whose entries are either d⁺ or d⁻, in locations corresponding to positive and negative examples. It is possible to prove that this technique is equivalent to controlling the size of the αᵢ in a way that depends on the size of the class, introducing a bias for larger αᵢ in the class with smaller d. This in turn corresponds to an asymmetric margin; i.e., the class with smaller d will be kept further away from the decision boundary. In some cases, the extreme imbalance of the two classes, along with the presence of noise, creates a situation in which points from the minority class can be easily mistaken for mislabeled points. Enforcing a strong bias against training errors in the minority class provides protection against such errors and forces the SVM to make the positive examples support vectors. Thus, choosing

$${d}^{+} = \frac{1}{{n}^{+}} and {d}^{-} = \frac{1}{{n}^{-}}$$

provides a heuristic way to automatically adjust the relative importance of the two classes, based on their respective cardinalities. This technique effectively controls the trade-off between sensitivity and specificity.

In the present invention, a linear kernel can be used. The similarity between two marker profiles X and Y can be the dot product X·Y. In one embodiment, the kernel is

K(X,Y)=X·Y+1

In another embodiment, a kernel of degree d is used

K(X,Y)=(X·Y+1)^{d}, where d can be either 2, 3, . . .

In still another embodiment, a Gaussian kernel is used

$$K ( X , Y ) = exp \frac{- {\vert X - Y \vert}^{2}}{2 {σ}^{2}}$$

where σ is the width of the Gaussian.

D. Logistic Regression

In some embodiments, the classifier is based on a regression model, preferably a logistic regression model. Such a regression model includes a coefficient for each of the molecular markers in a selected set of molecular biomarkers of the invention. In such embodiments, the coefficients for the regression model are computed using, for example, a maximum likelihood approach. In particular embodiments, molecular biomarker data from two different classification or phenotype groups, e.g., deregulated or regulated Wnt/β-catenin signaling pathway, response or non-response to treatment to an agent that modulates the Wnt/β-catenin signaling pathway, is used and the dependent variable is the phenotypic status of the patient for which molecular marker characteristic data are from.

Some embodiments of the present invention provide generalizations of the logistic regression model that handle multicategory (polychotomous) responses. Such embodiments can be used to discriminate an organism into one or three or more classification groups, e.g., good, intermediate, and poor therapeutic response to treatment with Wnt/β-catenin signaling pathway agents. Such regression models use multicategory logit models that simultaneously refer to all pairs of categories, and describe the odds of response in one category instead of another. Once the model specifies logits for a certain (J−1) pairs of categories, the rest are redundant. See, for example, Agresti, An Introduction to Categorical Data Analysis, John Wiley & Sons, Inc., 1996, New York, Chapter 8, which is hereby incorporated by reference.

E. Discriminant Analysis

Linear discriminant analysis (LDA) attempts to classify a subject into one of two categories based on certain object properties. In other words, LDA tests whether object attributes measured in an experiment predict categorization of the objects. LDA typically requires continuous independent variables and a dichotomous categorical dependent variable. In the present invention, the expression values for the selected set of molecular markers of the invention across a subset of the training population serve as the requisite continuous independent variables. The clinical group classification of each of the members of the training population serves as the dichotomous categorical dependent variable.

LDA seeks the linear combination of variables that maximizes the ratio of between-group variance and within-group variance by using the grouping information. Implicitly, the linear weights used by LDA depend on how the expression of a molecular biomarker across the training set separates in the two groups (e.g., a group that has deregulated Wnt/β-catenin signaling pathway and a group that have regulated Wnt/β-catenin signaling pathway status) and how this gene expression correlates with the expression of other genes. In some embodiments, LDA is applied to the data matrix of the N members in the training sample by K genes in a combination of genes described in the present invention. Then, the linear discriminant of each member of the training population is plotted. Ideally, those members of the training population representing a first subgroup (e.g. those subjects that have deregulated Wnt/β-catenin signaling pathway status) will cluster into one range of linear discriminant values (e.g., negative) and those member of the training population representing a second subgroup (e.g. those subjects that have regulated Wnt/β-catenin signaling pathway status) will cluster into a second range of linear discriminant values (e.g., positive). The LDA is considered more successful when the separation between the clusters of discriminant values is larger. For more information on linear discriminant analysis, see Duda, Pattern Classification, Second Edition, 2001, John Wiley & Sons, Inc; and Hastie, 2001, The Elements of Statistical Learning, Springer, N.Y.; Venables & Ripley, 1997, Modern Applied Statistics with s-plus, Springer, N.Y.

Quadratic discriminant analysis (QDA) takes the same input parameters and returns the same results as LDA. QDA uses quadratic equations, rather than linear equations, to produce results. LDA and QDA are interchangeable, and which to use is a matter of preference and/or availability of software to support the analysis. Logistic regression takes the same input parameters and returns the same results as LDA and QDA.

F. Decision Trees

In some embodiments of the present invention, decision trees are used to classify subjects using expression data for a selected set of molecular biomarkers of the invention. Decision tree algorithms belong to the class of supervised learning algorithms. The aim of a decision tree is to induce a classifier (a tree) from real-world example data. This tree can be used to classify unseen examples which have not been used to derive the decision tree.

A decision tree is derived from training data. An example contains values for the different attributes and what class the example belongs. In one embodiment, the training data is expression data for a combination of genes described in the present invention across the training population.

The following algorithm describes a decision tree derivation:

```
Tree (Examples, Class, Attributes)
    Create a root node
    If all Examples have the same Class value, give the root this label
    Else if Attributes is empty label the root according to the most common value
    Else begin
        Calculate the information gain for each attribute
        Select the attribute A with highest information gain and make this the root attribute
        For each possible value, v, of this attribute
            Add a new branch below the root, corresponding to A = v
            Let Examples(v) be those examples with A = v
            If Examples(v) is empty, make the new branch a leaf node labeled
with the  most common value among Examples
            Else let the new branch be the tree created by
                Tree(Examples(v),Class,Attributes - {A})
    end
```

A more detailed description of the calculation of information gain is shown in the following. If the possible classes vᵢ of the examples have probabilities P(vᵢ) then the information content I of the actual answer is given by:

$$I ( P ( {v}_{1} ) , … , P ( {v}_{n} ) ) = {\sum }_{i = 1}^{n} - P ( {v}_{i} ) {log}_{2} P ( {v}_{i} )$$

The I-value shows how much information we need in order to be able to describe the outcome of a classification for the specific dataset used. Supposing that the dataset contains p positive and n negative (examples (e.g. individuals), the information contained in a correct answer is:

$$I ( \frac{p}{p + n} , \frac{n}{p + n} ) = - \frac{p}{p + n} {log}_{2} \frac{p}{p + n} - \frac{n}{p + n} {log}_{2} \frac{n}{p + n}$$

where log₂ is the logarithm using base two. By testing single attributes the amount of information needed to make a correct classification can be reduced. The remainder for a specific attribute A (e.g. a gene biomarker) shows how much the information that is needed can be reduced.

$$Remainder ( A ) = {\sum }_{i = 1}^{v} \frac{{p}_{i} + {n}_{i}}{p + n} I ( \frac{{p}_{i}}{{p}_{i} + {n}_{i}} , \frac{{n}_{i}}{{p}_{i} + {n}_{i}} )$$

“v” is the number of unique attribute values for attribute A in a certain dataset, “i” is a certain attribute value, “pᵢ” is the number of examples for attribute A where the classification is positive, “nᵢ” is the number of examples for attribute A where the classification is negative.

The information gain of a specific attribute A is calculated as the difference between the information content for the classes and the remainder of attribute A:

$$Gain ( A ) = I ( \frac{p}{p + n} , \frac{n}{p + n} ) - Remainder ( A )$$

The information gain is used to evaluate how important the different attributes are for the classification (how well they split up the examples), and the attribute with the highest information.

In general there are a number of different decision tree algorithms, many of which are described in Duda, Pattern Classification, Second Edition, 2001, John Wiley & Sons, Inc. Decision tree algorithms often require consideration of feature processing, impurity measure, stopping criterion, and pruning. Specific decision tree algorithms include, cut are not limited to classification and regression trees (CART), multivariate decision trees, ID3, and C4.5.

In one approach, when an exemplary embodiment of a decision tree is used, the gene expression data for a selected set of molecular markers of the invention across a training population is standardized to have mean zero and unit variance. The members of the training population are randomly divided into a training set and a test set. For example, in one embodiment, two thirds of the members of the training population are placed in the training set and one third of the members of the training population are placed in the test set. The expression values for a select combination of genes described in the present invention is used to construct the decision tree. Then, the ability for the decision tree to correctly classify members in the test set is determined. In some embodiments, this computation is performed several times for a given combination of molecular markers. In each iteration of the computation, the members of the training population are randomly assigned to the training set and the test set. Then, the quality of the combination of molecular markers is taken as the average of each such iteration of the decision tree computation.

G. Clustering

In some embodiments, the expression values for a selected set of molecular markers of the invention are used to cluster a training set. For example, consider the case in which ten gene biomarkers described in one of the geneses of the present invention are used. Each member m of the training population will have expression values for each of the ten biomarkers. Such values from a member m in the training population define the vector:

| | | | | | | | | | |
|---|---|---|---|---|---|---|---|---|---|
| X₁ₘ | X₂ₘ | X₃ₘ | X₄ₘ | X₅ₘ | X₆ₘ | X₇ₘ | X₈ₘ | X₉ₘ | X₁₀ₘ |

where Xᵢₘ is the expression level of the i^{th} gene in organism m. If there are m organisms in the training set, selection of i genes will define m vectors. Note that the methods of the present invention do not require that each the expression value of every single gene used in the vectors be represented in every single vector m. In other words, data from a subject in which one of the i^{th} genes is not found can still be used for clustering. In such instances, the missing expression value is assigned either a “zero” or some other normalized value. In some embodiments, prior to clustering, the gene expression values are normalized to have a mean value of zero and unit variance.

Those members of the training population that exhibit similar expression patterns across the training group will tend to cluster together. A particular combination of genes of the present invention is considered to be a good classifier in this aspect of the invention when the vectors cluster into the trait groups found in the training population. For instance, if the training population includes patients with good or poor prognosis, a clustering classifier will cluster the population into two groups, with each group uniquely representing either a deregulated Wnt/β-catenin signalling pathway status or a regulated Wnt/β-catenin signalling pathway status.

Clustering is described on pages 211-256 of Duda and Hart, Pattern Classification and Scene Analysis, 1973, John Wiley & Sons, Inc., New York. As described in Section 6.7 of Duda, the clustering problem is described as one of finding natural groupings in a dataset. To identify natural groupings, two issues are addressed. First, a way to measure similarity (or dissimilarity) between two samples is determined. This metric (similarity measure) is used to ensure that the samples in one cluster are more like one another than they are to samples in other clusters. Second, a mechanism for partitioning the data into clusters using the similarity measure is determined.

Similarity measures are discussed in Section 6.7 of Duda, where it is stated that one way to begin a clustering investigation is to define a distance function and to compute the matrix of distances between all pairs of samples in a dataset. If distance is a good measure of similarity, then the distance between samples in the same cluster will be significantly less than the distance between samples in different clusters. However, as stated on page 215 of Duda, clustering does not require the use of a distance metric. For example, a nonmetric similarity function s(x, x′) can be used to compare two vectors x and x′. Conventionally, s(x, x′) is a symmetric function whose value is large when x and x′ are somehow “similar”. An example of a nonmetric similarity function s(x, x′) is provided on page 216 of Duda.

Once a method for measuring “similarity” or “dissimilarity” between points in a dataset has been selected, clustering requires a criterion function that measures the clustering quality of any partition of the data. Partitions of the data set that extremize the criterion function are used to cluster the data. See page 217 of Duda. Criterion functions are discussed in Section 6.8 of Duda.

More recently, Duda et al., Pattern Classification, 2^{nd} edition, John Wiley & Sons, Inc. New York, has been published. Pages 537-563 describe clustering in detail. More information on clustering techniques can be found in Kaufman and Rousseeuw, 1990, Finding Groups in Data: An Introduction to Cluster Analysis, Wiley, New York, N.Y.; Everitt, 1993, Cluster analysis (3d ed.), Wiley, New York, N.Y.; and Backer, 1995, Computer-Assisted Reasoning in Cluster Analysis, Prentice Hall, Upper Saddle River, N.J. Particular exemplary clustering techniques that can be used in the present invention include, but are not limited to, hierarchical clustering (agglomerative clustering using nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering.

H. Principal Component Analysis

Principal component analysis (PCA) has been proposed to analyze gene expression data. Principal component analysis is a classical technique to reduce the dimensionality of a data set by transforming the data to a new set of variable (principal components) that summarize the features of the data. See, for example, Jolliffe, 1986, Principal Component Analysis, Springer, N.Y. Principal components (PCs) are uncorrelate and are ordered such that the k^{th} PC has the kth largest variance among PCs. The k^{th} PC can be interpreted as the direction that maximizes the variation of the projections of the data points such that it is orthogonal to the first k−1 PCs. The first few PCs capture most of the variation in the data set. In contrast, the last few PCs are often assumed to capture only the residual ‘noise’ in the data.

PCA can also be used to create a classifier in accordance with the present invention. In such an approach, vectors for a selected set of molecular biomarkers of the invention can be constructed in the same manner described for clustering above. In fact, the set of vectors, where each vector represents the expression values for the select genes from a particular member of the training population, can be considered a matrix. In some embodiments, this matrix is represented in a Free-Wilson method of qualitative binary description of monomers (Kubinyi, 1990, 3D QSAR in drug design theory methods and applications, Pergamon Press, Oxford, pp 589-638), and distributed in a maximally compressed space using PCA so that the first principal component (PC) captures the largest amount of variance information possible, the second principal component (PC) captures the second largest amount of all variance information, and so forth until all variance information in the matrix has been accounted for.

Then, each of the vectors (where each vector represents a member of the training population) is plotted. Many different types of plots are possible. In some embodiments, a one-dimensional plot is made. In this one-dimensional plot, the value for the first principal component from each of the members of the training population is plotted. In this form of plot, the expectation is that members of a first group will cluster in one range of first principal component values and members of a second group will cluster in a second range of first principal component values.

In one example, the training population comprises two classification groups. The first principal component is computed using the molecular biomarker expression values for the select genes of the present invention across the entire training population data set where the classification outcomes are known. Then, each member of the training set is plotted as a function of the value for the first principal component. In this example, those members of the training population in which the first principal component is positive represent one classification outcome and those members of the training population in which the first principal component is negative represent the other classification outcome.

In some embodiments, the members of the training population are plotted against more than one principal component. For example, in some embodiments, the members of the training population are plotted on a two-dimensional plot in which the first dimension is the first principal component and the second dimension is the second principal component. In such a two-dimensional plot, the expectation is that members of each subgroup represented in the training population will cluster into discrete groups. For example, a first cluster of members in the two-dimensional plot will represent subjects in the first classification group, a second cluster of members in the two-dimensional plot will represent subjects in the second classification group, and so forth.

In some embodiments, the members of the training population are plotted against more than two principal components and a determination is made as to whether the members of the training population are clustering into groups that each uniquely represents a subgroup found in the training population. In some embodiments, principal component analysis is performed by using the R mva package (Anderson, 1973, Cluster Analysis for applications, Academic Press, New York 1973; Gordon, Classification, Second Edition, Chapman and Hall, CRC, 1999.). Principal component analysis is further described in Duda, Pattern Classification, Second Edition, 2001, John Wiley & Sons, Inc.

I. Nearest Neighbor Classifier Analysis

Nearest neighbor classifiers are memory-based and require no model to be fit. Given a query point x₀, the k training points x₍ᵣ₎, r, . . . , k closest in distance to x₀ are identified and then the point x₀ is classified using the k nearest neighbors. Ties can be broken at random. In some embodiments, Euclidean distance in feature space is used to determine distance as:

d₍ᵢ₎=∥x₍ᵢ₎−xₒ∥.

Typically, when the nearest neighbor algorithm is used, the expression data used to compute the linear discriminant is standardized to have mean zero and variance 1. In the present invention, the members of the training population are randomly divided into a training set and a test set. For example, in one embodiment, two thirds of the members of the training population are placed in the training set and one third of the members of the training population are placed in the test set. Profiles of a selected set of molecular biomarkers of the invention represents the feature space into which members of the test set are plotted. Next, the ability of the training set to correctly characterize the members of the test set is computed. In some embodiments, nearest neighbor computation is performed several times for a given combination of genes of the present invention. In each iteration of the computation, the members of the training population are randomly assigned to the training set and the test set. Then, the quality of the combination of genes is taken as the average of each such iteration of the nearest neighbor computation.

The nearest neighbor rule can be refined to deal with issues of unequal class priors, differential misclassification costs, and feature selection. Many of these refinements involve some form of weighted voting for the neighbors. For more information on nearest neighbor analysis, see Duda, Pattern Classification, Second Edition, 2001, John Wiley & Sons, Inc; and Hastie, 2001, The Elements of Statistical Learning, Springer, N.Y.

J. Evolutionary Methods

Inspired by the process of biological evolution, evolutionary methods of classifier design employ a stochastic search for an optimal classifier. In broad overview, such methods create several classifiers—a population—from measurements of gene products of the present invention. Each classifier varies somewhat from the other. Next, the classifiers are scored on expression data across the training population. In keeping with the analogy with biological evolution, the resulting (scalar) score is sometimes called the fitness. The classifiers are ranked according to their score and the best classifiers are retained (some portion of the total population of classifiers). Again, in keeping with biological terminology, this is called survival of the fittest. The classifiers are stochastically altered in the next generation—the children or offspring. Some offspring classifiers will have higher scores than their parent in the previous generation, some will have lower scores. The overall process is then repeated for the subsequent generation: The classifiers are scored and the best ones are retained, randomly altered to give yet another generation, and so on. In part, because of the ranking, each generation has, on average, a slightly higher score than the previous one. The process is halted when the single best classifier in a generation has a score that exceeds a desired criterion value. More information on evolutionary methods is found in, for example, Duda, Pattern Classification, Second Edition, 2001, John Wiley & Sons, Inc.

K. Bagging, Boosting and the Random Subspace Method

Bagging, boosting and the random subspace method are combining techniques that can be used to improve weak classifiers. These techniques are designed for, and usually applied to, decision trees. In addition, Skurichina and Duin provide evidence to suggest that such techniques can also be useful in linear discriminant analysis.

In bagging, one samples the training set, generating random independent bootstrap replicates, constructs the classifier on each of these, and aggregates them by a simple majority vote in the final decision rule. See, for example, Breiman, 1996, Machine Learning 24, 123-140; and Efron & Tibshirani, An Introduction to Bootstrap, Chapman & Hall, New York, 1993.

In boosting, classifiers are constructed on weighted versions of the training set, which are dependent on previous classification results. Initially, all objects have equal weights, and the first classifier is constructed on this data set. Then, weights are changed according to the performance of the classifier. Erroneously classified objects (molecular biomarkers in the data set) get larger weights, and the next classifier is boosted on the reweighted training set. In this way, a sequence of training sets and classifiers is obtained, which is then combined by simple majority voting or by weighted majority voting in the final decision. See, for example, Freund & Schapire, “Experiments with a new boosting algorithm,” Proceedings 13^{th} International Conference on Machine Learning, 1996, 148-156.

To illustrate boosting, consider the case where there are two phenotypic groups exhibited by the population under study, phenotype 1, and phenotype 2. Given a vector of molecular markers X, a classifier G(X) produces a prediction taking one of the type values in the two value set: {phenotype 1, phenotype 2}. The error rate on the training sample is

$$\overline{err} = \frac{1}{N} {\sum }_{i = 1}^{N} I ( {y}_{i} \neq G ( {x}_{i} ) )$$

where N is the number of subjects in the training set (the sum total of the subjects that have either phenotype 1 or phenotype 2).

A weak classifier is one whose error rate is only slightly better than random guessing. In the boosting algorithm, the weak classification algorithm is repeatedly applied to modified versions of the data, thereby producing a sequence of weak classifiers Gₘ(x), m, =1, 2, . . . , M. The predictions from all of the classifiers in this sequence are then combined through a weighted majority vote to produce the final prediction:

$$G ( x ) = sign ( {\sum }_{m = 1}^{M} {α}_{m} {G}_{m} ( x ) )$$

Here α₁, α₂, αₘ are computed by the boosting algorithm and their purpose is to weigh the contribution of each respective Gₘ(x). Their effect is to give higher influence to the more accurate classifiers in the sequence.

The data modifications at each boosting step consist of applying weights w₁, w₂, . . . , wₙ to each of the training observations (xᵢ, yᵢ), i=1, 2, . . . , N. Initially all the weights are set to wᵢ=1/N, so that the first step simply trains the classifier on the data in the usual manner. For each successive iteration m=2, 3, . . . , M the observation weights are individually modified and the classification algorithm is reapplied to the weighted observations. At stem in, those observations that were misclassified by the classifier Gₘ₋₁(x) induced at the previous step have their weights increased, whereas the weights are decreased for those that were classified correctly. Thus as iterations proceed, observations that are difficult to correctly classify receive ever-increasing influence. Each successive classifier is thereby forced to concentrate on those training observations that are missed by previous ones in the sequence.

The exemplary boosting algorithm is summarized as follows:

1. Initialize the observation weights wᵢ = 1/N, i = 1, 2, . . ., N.
2. For m = 1 to M:

(a) Fit a classifier Gₘ(x) to the training set using weights wᵢ.
(b) Compute

${err}_{m} = \frac{{\sum }_{i = 1}^{N} {w}_{i} I ( {y}_{i} \neq {G}_{m} ( {x}_{i} ) )}{{\sum }_{i = 1}^{N} {w}_{i}}$

(c) Compute αₘ = log((1 − errₘ)/errₘ).
(d) Set wᵢ ← wᵢ · exp[αₘ · I(yᵢ ≠ Gₘ(xᵢ))], i = 1, 2, . . ., N.

$3. Output G ( x ) = sign ⌊ {\sum }_{m = 1}^{M} {α}_{m} {G}_{m} ( x ) ⌋$

In the algorithm, the current classifier Gₘ(x) is induced on the weighted observations at line 2a. The resulting weighted error rate is computed at line 2b. Line 2c calculates the weight αₘ given to Gₘ(x) in producing the final classifier G(x) (line 3). The individual weights of each of the observations are updated for the next iteration at line 2d. Observations misclassified by Gₘ(x) have their weights scaled by a factor exp(αₘ), increasing their relative influence for inducing the next classifier Gₘ₊₁(x) in the sequence. In some embodiments, modifications of the Freund and Schapire, 1997, Journal of Computer and System Sciences 55, pp. 119-139, boosting method are used. See, for example, Hasti et al., The Elements of Statistical Learning, 2001, Springer, N.Y., Chapter 10. In some embodiments, boosting or adaptive boosting methods are used.

In some embodiments, modifications of Freund and Schapire, 1997, Journal of Computer and System Sciences 55, pp. 119-139, are used. For example, in some embodiments, feature pre-selection is performed using a technique such as the nonparametric scoring methods of Park et al., 2002, Pac. Symp. Biocomput. 6, 52-63. Feature pre-selection is a form of dimensionality reduction in which the genes that discriminate between classifications the best are selected for use in the classifier. Then, the LogitBoost procedure introduced by Friedman et al., 2000, Ann Stat 28, 337-407 is used rather than the boosting procedure of Freund and Schapire. In some embodiments, the boosting and other classification methods of Ben-Dor et al., 2000, Journal of Computational Biology 7, 559-583 are used in the present invention. In some embodiments, the boosting and other classification methods of Freund and Schapire, 1997, Journal of Computer and System Sciences 55, 119-139, are used.

In the random subspace method, classifiers are constructed in random subspaces of the data feature space. These classifiers are usually combined by simple majority voting in the final decision rule. See, for example, Ho, “The Random subspace method for constructing decision forests,” IEEE Trans Pattern Analysis and Machine Intelligence, 1998; 20(8): 832-844.

L. Other Algorithms

The pattern classification and statistical techniques described above are merely examples of the types of models that can be used to construct a model for classification. Moreover, combinations of the techniques described above can be used. Some combinations, such as the use of the combination of decision trees and boosting, have been described. However, many other combinations are possible. In addition, in other techniques in the art such as Projection Pursuit and Weighted Voting can be used to construct a classifier.

### 3.5 DETERMINATION OF BIOMARKER GENE EXPRESSION LEVELS

### 3.5.1 Methods

The expression levels of the biomarker genes in a sample may be determined by any means known in the art. The expression level may be determined by isolating and determining the level (i.e., amount) of nucleic acid transcribed from each biomarker gene. Alternatively, or additionally, the level of specific proteins translated from mRNA transcribed from a biomarker gene may be determined.

The level of expression of specific biomarker genes can be accomplished by determining the amount of mRNA, or polynucleotides derived therefrom, present in a sample. Any method for determining RNA levels can be used. For example, RNA is isolated from a sample and separated on an agarose gel. The separated RNA is then transferred to a solid support, such as a filter. Nucleic acid probes representing one or more biomarkers are then hybridized to the filter by northern hybridization, and the amount of biomarker-derived RNA is determined. Such determination can be visual, or machine-aided, for example, by use of a densitometer. Another method of determining RNA levels is by use of a dot-blot or a slot-blot. In this method, RNA, or nucleic acid derived therefrom, from a sample is labeled. The RNA or nucleic acid derived therefrom is then hybridized to a filter containing oligonucleotides derived from one or more biomarker genes, wherein the oligonucleotides are placed upon the filter at discrete, easily-identifiable locations. Hybridization, or lack thereof; of the labeled RNA to the filter-bound oligonucleotides is determined visually or by densitometer. Polynucleotides can be labeled using a radiolabel or a fluorescent (i.e., visible) label.

These examples are not intended to be limiting. Other methods of determining RNA abundance are known in the art, including, but not limited to quantitative PCR methods, such as TAQMAN®, and Nanostring's NCOUNTER™ Digital Gene Expression System (Seattle, Wash.) (See also WO2007076128; WO2007076129).

The level of expression of particular biomarker genes may also be assessed by determining the level of the specific protein expressed from the biomarker genes. This can be accomplished, for example, by separation of proteins from a sample on a polyacrylamide gel, followed by identification of specific biomarker-derived proteins using antibodies in a western blot. Alternatively, proteins can be separated by two-dimensional gel electrophoresis systems. Two-dimensional gel electrophoresis is well-known in the art and typically involves isoelectric focusing along a first dimension followed by SDS-PAGE electrophoresis along a second dimension. See, e.g., Flames et al, 1990, GEL ELECTROPHORESIS OF PROTEINS: A PRACTICAL APPROACH, IRL Press, New York; Shevchenko et al., Proc. Nat'l Acad. Sci. USA 93:1440-1445 (1996); Sagliocco et al., Yeast 12:1519-1533 (1996); Lander, Science 274:536-539 (1996). The resulting electropherograms can be analyzed by numerous techniques, including mass spectrometric techniques, western blotting and immunoblot analysis using polyclonal and monoclonal antibodies.

Alternatively, biomarker-derived protein levels can be determined by constructing an antibody microarray in which binding sites comprise immobilized, preferably monoclonal, antibodies specific to a plurality of protein species encoded by the cell genome. Preferably, antibodies are present for a substantial fraction of the biomarker-derived proteins of interest. Methods for making monoclonal antibodies are well known (see, e.g., Harlow and Lane, 1988, ANTIBODIES: A LABORATORY MANUAL, Cold Spring Harbor, N.Y., which is incorporated in its entirety for all purposes). In one embodiment, monoclonal antibodies are raised against synthetic peptide fragments designed based on genomic sequence of the cell. With such an antibody array, proteins from the cell are contacted to the array, and their binding is assayed with assays known in the art. Generally, the expression, and the level of expression, of proteins of diagnostic or prognostic interest can be detected through immunohistochemical staining of tissue slices or sections.

Finally, expression of biomarker genes in a number of tissue specimens may be characterized using a “tissue array” (Kononen et al., Nat. Med. 4(7):844-7 (1998)). In a tissue array, multiple tissue samples are assessed on the same microarray. The arrays allow in situ detection of RNA and protein levels; consecutive sections allow the analysis of multiple samples simultaneously.

### 3.5.2 Microarrays

In preferred embodiments, polynucleotide microarrays are used to measure expression so that the expression status of each of the biomarkers above is assessed simultaneously. In a specific embodiment, the invention provides for oligonucleotide or cDNA arrays comprising probes hybridizable to the genes corresponding to each of the biomarker sets described above (i.e., biomarkers to determine the molecular type or subtype of a tumor; biomarkers to classify the Wnt/β-catenin pathway signaling status of a tumor; biomarkers to predict response of a subject to an agent that modulates the Wnt/β-catenin signaling pathway; biomarkers to measure pharmacodynamic effect of a therapeutic agent on the Wnt/β-catenin signaling pathway).

The microarrays provided by the present invention may comprise probes hybridizable to the genes corresponding to biomarkers able to distinguish the status of one, two, or all three of the clinical conditions noted above. In particular, the invention provides polynucleotide arrays comprising probes to a subset or subsets of at least 5, 10, 20, 30, 40, 50, 100 genetic biomarkers, up to the full set of 38 biomarkers, which distinguish Wnt/β-catenin signaling pathway deregulated and regulated subjects or tumors.

In yet another specific embodiment, microarrays that are used in the methods disclosed herein optionally comprise biomarkers additional to at least some of the biomarkers listed in Table 5. For example, in a specific embodiment, the microarray is a screening or scanning array as described in Altschuler et al., International Publication WO 02/18646, published Mar. 7, 2002 and Scherer et al., International Publication WO 02/16650, published Feb. 28, 2002. The scanning and screening arrays comprise regularly-spaced, positionally-addressable probes derived from genomic nucleic acid sequence, both expressed and unexpressed. Such arrays may comprise probes corresponding to a subset of, or all of, the biomarkers listed in Table 3, or a subset thereof as described above, and can be used to monitor biomarker expression in the same way as a microarray containing only biomarkers listed in Table 3.

In yet another specific embodiment, the microarray is a commercially-available cDNA microarray that comprises at least five of the biomarkers listed in Table 5. Preferably, a commercially-available cDNA microarray comprises all of the biomarkers listed in Table 5. However, such a microarray may comprise 5, 10, 15, 25, 50, 100 or more of the biomarkers in any of Table 5, up to the maximum number of biomarkers in a Table 5, and may comprise all of the biomarkers in any one of Table 5 and a subset of another of Table 5, or subsets of each as described above. In a specific embodiment of the microarrays used in the methods disclosed herein, the biomarkers that are all or a portion of Table 5 make up at least 50%, 60%, 70%, 80%, 90%, 95% or 98% of the probes on the microarray.

General methods pertaining to the construction of microarrays comprising the biomarker sets and/or subsets above are described in the following sections.

### 3.5.2.1 Construction of Microarrays

Microarrays are prepared by selecting probes which comprise a polynucleotide sequence, and then immobilizing such probes to a solid support or surface. For example, the probes may comprise DNA sequences, RNA sequences, or copolymer sequences of DNA and RNA. The polynucleotide sequences of the probes may also comprise DNA and/or RNA analogues, or combinations thereof. For example, the polynucleotide sequences of the probes may be full or partial fragments of genomic DNA. The polynucleotide sequences of the probes may also be synthesized nucleotide sequences, such as synthetic oligonucleotide sequences. The probe sequences can be synthesized either enzymatically in vivo, enzymatically in vitro (e.g., by PCR), or non-enzymatically in vitro.

The probe or probes used in the methods of the invention are preferably immobilized to a solid support which may be either porous or non-porous. For example, the probes of the invention may be polynucleotide sequences which are attached to a nitrocellulose or nylon membrane or filter covalently at either the 3′ or the 5′ end of the polynucleotide. Such hybridization probes are well known in the art (see, e.g., Sambrook et al., MOLECULAR CLONING—A LABORATORY MANUAL (2ND ED.), Vols. 1-3, Cold Spring Harbor Laboratory, Cold Spring Harbor, N.Y. (1989). Alternatively, the solid support or surface may be a glass or plastic surface. In a particularly preferred embodiment, hybridization levels are measured to microarrays of probes consisting of a solid phase on the surface of which are immobilized a population of polynucleotides, such as a population of DNA or DNA mimics, or, alternatively, a population of RNA or RNA mimics. The solid phase may be a nonporous or, optionally, a porous material such as a gel.

In preferred embodiments, a microarray comprises a support or surface with an ordered array of binding (e.g., hybridization) sites or “probes” each representing one of the biomarkers described herein. Preferably the microarrays are addressable arrays, and more preferably positionally addressable arrays. More specifically, each probe of the array is preferably located at a known, predetermined position on the solid support such that the identity (i.e., the sequence) of each probe can be determined from its position in the array (i.e., on the support or surface). In preferred embodiments, each probe is covalently attached to the solid support at a single site.

Microarrays can be made in a number of ways, of which several are described below. However produced, microarrays share certain characteristics. The arrays are reproducible, allowing multiple copies of a given array to be produced and easily compared with each other. Preferably, microarrays are made from materials that are stable under binding (e.g., nucleic acid hybridization) conditions. The microarrays are preferably small, e.g., between 1 cm² and 25 cm², between 12 cm² and 13 cm², or 3 cm². However, larger arrays are also contemplated and may be preferable, e.g., for use in screening arrays. Preferably, a given binding site or unique set of binding sites in the microarray will specifically bind (e.g., hybridize) to the product of a single gene in a cell (e.g., to a specific mRNA, or to a specific cDNA derived therefrom). However, in general, other related or similar sequences will cross hybridize to a given binding site.

The microarrays of the present invention include one or more test probes, each of which has a polynucleotide sequence that is complementary to a subsequence of RNA or DNA to be detected. Preferably, the position of each probe on the solid surface is known. Indeed, the microarrays are preferably positionally addressable arrays. Specifically, each probe of the array is preferably located at a known, predetermined position on the solid support such that the identity (i.e., the sequence) of each probe can be determined from its position on the array (i.e., on the support or surface).

According to the invention, the microarray is an array (i.e., a matrix) in which each position represents one of the biomarkers described herein. For example, each position can contain a DNA or DNA analogue based on genomic DNA to which a particular RNA or cDNA transcribed from that genetic biomarker can specifically hybridize. The DNA or DNA analogue can be, e.g., a synthetic oligomer or a gene fragment. In one embodiment, probes representing each of the biomarkers is present on the array.

### 3.5.2.2 Preparing Probes for Microarrays

As noted above, the “probe” to which a particular polynucleotide molecule specifically hybridizes according to the invention contains a complementary genomic polynucleotide sequence. The probes of the microarray preferably consist of nucleotide sequences of no more than 1,000 nucleotides. In some embodiments, the probes of the array consist of nucleotide sequences of 10 to 1,000 nucleotides. In a preferred embodiment, the nucleotide sequences of the probes are in the range of 10-200 nucleotides in length and are genomic sequences of a species of organism, such that a plurality of different probes is present, with sequences complementary and thus capable of hybridizing to the genome of such a species of organism, sequentially tiled across all or a portion of such genome. In other specific embodiments, the probes are in the range of 10-30 nucleotides in length, in the range of 10-40 nucleotides in length, in the range of 20-50 nucleotides in length, in the range of 40-80 nucleotides in length, in the range of 50-150 nucleotides in length, in the range of 80-120 nucleotides in length, and most preferably are 60 nucleotides in length.

The probes may comprise DNA or DNA “mimics” (e.g., derivatives and analogues) corresponding to a portion of an organism's genome. In another embodiment, the probes of the microarray are complementary RNA or RNA mimics. DNA mimics are polymers composed of subunits capable of specific, Watson-Crick-like hybridization with DNA, or of specific hybridization with RNA. The nucleic acids can be modified at the base moiety, at the sugar moiety, or at the phosphate backbone. Exemplary DNA mimics include, e.g., phosphorothioates.

DNA can be obtained, e.g., by polymerase chain reaction (PCR) amplification of genomic DNA or cloned sequences. PCR primers are preferably chosen based on a known sequence of the genome that will result in amplification of specific fragments of genomic DNA. Computer programs that are well known in the art are useful in the design of primers with the required specificity and optimal amplification properties, such as Oligo version 5.0 (National Biosciences). Typically each probe on the microarray will be between 10 bases and 50,000 bases, usually between 300 bases and 1,000 bases in length. PCR methods are well known in the art, and are described, for example, in Innis et al., eds., PCR PROTOCOLS: A GUIDE TO METHODS AND APPLICATIONS, Academic Press Inc., San Diego, Calif. (1990). It will be apparent to one skilled in the art that controlled robotic systems are useful for isolating and amplifying nucleic acids.

An alternative, preferred means for generating the polynucleotide probes of the microarray is by synthesis of synthetic polynucleotides or oligonucleotides, e.g., using N-phosphonate or phosphoramidite chemistries (Froehler et al., Nucleic Acid Res. 14:5399-5407 (1986); McBride et al., Tetrahedron Lett. 24:246-248 (1983)). Synthetic sequences are typically between about 10 and about 500 bases in length, more typically between about 20 and about 100 bases, and most preferably between about 40 and about 70 bases in length. In some embodiments, synthetic nucleic acids include non-natural bases, such as, but by no means limited to, inosine. As noted above, nucleic acid analogues may be used as binding sites for hybridization. An example of a suitable nucleic acid analogue is peptide nucleic acid (see, e.g., Egholm et al., Nature 363:566-568 (1993); U.S. Pat. No. 5,539,083). Probes are preferably selected using an algorithm that takes into account binding energies, base composition, sequence complexity, cross-hybridization binding energies, and secondary structure (see Friend et al., International Patent Publication WO 01/05935, published Jan. 25, 2001; Hughes et al., Nat. Biotech. 19:342-7 (2001)).

A skilled artisan will also appreciate that positive control probes, e.g., probes known to be complementary and hybridizable to sequences in the target polynucleotide molecules, and negative control probes, e.g., probes known to not be complementary and hybridizable to sequences in the target polynucleotide molecules, should be included on the array. In one embodiment, positive controls are synthesized along the perimeter of the array. In another embodiment, positive controls are synthesized in diagonal stripes across the array. In still another embodiment, the reverse complement for each probe is synthesized next to the position of the probe to serve as a negative control. In yet another embodiment, sequences from other species of organism are used as negative controls or as “spike-in” controls.

### 3.5.2.3 Attaching Probes to the Solid Surface

The probes are attached to a solid support or surface, which may be made, e.g., from glass, plastic (e.g., polypropylene, nylon), polyacrylamide, nitrocellulose, gel, or other porous or nonporous material. A preferred method for attaching the nucleic acids to a surface is by printing on glass plates, as is described generally by Schena et al, Science 270:467-470 (1995). This method is especially useful for preparing microarrays of cDNA (See also, DeRisi et al, Nature Genetics 14:457-460 (1996); Shalon et al., Genome Res. 6:639-645 (1996); and Schena et al., Proc. Natl. Acad. Sci. U.S.A. 93:10539-11286 (1995)).

A second preferred method for making microarrays is by making high-density oligonucleotide arrays. Techniques are known for producing arrays containing thousands of oligonucleotides complementary to defined sequences, at defined locations on a surface using photolithographic techniques for synthesis in situ (see, Fodor et al., 1991, Science 251:767-773; Pease et al, 1994, Proc. Natl. Acad. Sci. U.S.A. 91:5022-5026; Lockhart et al., 1996, Nature Biotechnology 14:1675; U.S. Pat. Nos. 5,578,832; 5,556,752; and 5,510,270) or other methods for rapid synthesis and deposition of defined oligonucleotides (Blanchard et al., Biosensors & Bioelectronics 11:687-690). When these methods are used, oligonucleotides (e.g., 60-mers) of known sequence are synthesized directly on a surface such as a derivatized glass slide. Usually, the array produced is redundant, with several oligonucleotide molecules per RNA.

Other methods for making microarrays, e.g., by masking (Maskos and Southern, 1992, Nuc. Acids. Res. 20:1679-1684), may also be used. In principle, and as noted supra, any type of array, for example, dot blots on a nylon hybridization membrane (see Sambrook et al., MOLECULAR CLONING—A LABORATORY MANUAL (2ND ED.), Vols. 1-3, Cold Spring Harbor Laboratory, Cold Spring Harbor, N.Y. (1989)) could be used. However, as will be recognized by those skilled in the art, very small arrays will frequently be preferred because hybridization volumes will be smaller.

In one embodiment, the arrays of the present invention are prepared by synthesizing polynucleotide probes on a support. In such an embodiment, polynucleotide probes are attached to the support covalently at either the 3′ or the 5′ end of the polynucleotide.

In a particularly preferred embodiment, microarrays of the invention are manufactured by means of an ink jet printing device for oligonucleotide synthesis, e.g., using the methods and systems described by Blanchard in U.S. Pat. No. 6,028,189; Blanchard et al., 1996, Biosensors and Bioelectronics 11:687-690; Blanchard, 1998, in SYNTHETIC DNA ARRAYS IN GENETIC ENGINEERING, Vol. 20, J. K. Setlow, Ed., Plenum Press, New York at pages 111-123. Specifically, the oligonucleotide probes in such microarrays are preferably synthesized in arrays, e.g., on a glass slide, by serially depositing individual nucleotide bases in “microdroplets” of a high surface tension solvent such as propylene carbonate. The microdroplets have small volumes (e.g., 100 pL or less, more preferably 50 pL or less) and are separated from each other on the microarray (e.g., by hydrophobic domains) to form circular surface tension wells which define the locations of the array elements (i.e., the different probes). Microarrays manufactured by this ink-jet method are typically of high density, preferably having a density of at least about 2,500 different probes per 1 cm². The polynucleotide probes are attached to the support covalently at either the 3′ or the 5′ end of the polynucleotide.

### 3.5.2.4 Target Polynucleotide Molecules

The polynucleotide molecules which may be analyzed by the present invention (the “target polynucleotide molecules”) may be from any clinically relevant source, but are expressed RNA or a nucleic acid derived therefrom (e.g., cDNA or amplified RNA derived from cDNA that incorporates an RNA polymerase promoter), including naturally occurring nucleic acid molecules, as well as synthetic nucleic acid molecules. In one embodiment, the target polynucleotide molecules comprise RNA, including, but by no means limited to, total cellular RNA, poly(A)+ messenger RNA (mRNA) or fraction thereof, cytoplasmic mRNA, or RNA transcribed from cDNA (i.e., cRNA; see, e.g., Linsley & Schelter, U.S. patent application Ser. No. 09/411,074, filed Oct. 4, 1999, or U.S. Pat. Nos. 5,545,522, 5,891,636, or 5,716,785). Methods for preparing total and poly(A)+ RNA are well known in the art, and are described generally, e.g., in Sambrook et al., MOLECULAR CLONING—A LABORATORY MANUAL (2ND ED.), Vols. 1-3, Cold Spring Harbor Laboratory, Cold Spring Harbor, N.Y. (1989). In one embodiment, RNA is extracted from cells of the various types of interest in this invention using guanidinium thiocyanate lysis followed by CsCl centrifugation (Chirgwin et al., 1979, Biochemistry 18:5294-5299). In another embodiment, total RNA is extracted using a silica gel-based column, commercially available examples of which include RNeasy (Qiagen, Valencia, Calif.) and StrataPrep (Stratagene, La Jolla, Calif.). In an alternative embodiment, which is preferred for S. cerevisiae, RNA is extracted from cells using phenol and chloroform, as described in Ausubel et al., eds., 1989, CURRENT PROTOCOLS IN MOLECULAR BIOLOGY, Vol. III, Green Publishing Associates, Inc., John Wiley & Sons, Inc., New York, at pp. 13.12.1-13.12.5). Poly(A)+ RNA can be selected, e.g., by selection with oligo-dT cellulose or, alternatively, by oligo-dT primed reverse transcription of total cellular RNA. In one embodiment, RNA can be fragmented by methods known in the art, e.g., by incubation with ZnCl₂, to generate fragments of RNA. In another embodiment, the polynucleotide molecules analyzed by the invention comprise cDNA, or PCR products of amplified RNA or cDNA.

In one embodiment, total RNA, mRNA, or nucleic acids derived therefrom, is isolated from a sample taken from a person afflicted with cancer. Target polynucleotide molecules that are poorly expressed in particular cells may be enriched using normalization techniques (Bonaldo et al., 1996, Genome Res. 6:791-806).

As described above, the target polynucleotides are detectably labeled at one or more nucleotides. Any method known in the art may be used to detectably label the target polynucleotides. Preferably, this labeling incorporates the label uniformly along the length of the RNA, and more preferably, the labeling is carried out at a high degree of efficiency. One embodiment for this labeling uses oligo-dT primed reverse transcription to incorporate the label; however, conventional methods of this method are biased toward generating 3′ end fragments. Thus, in a preferred embodiment, random primers (e.g., 9-mers) are used in reverse transcription to uniformly incorporate labeled nucleotides over the fill length of the target polynucleotides. Alternatively, random primers may be used in conjunction with PCR methods or T7 promoter-based in vitro transcription methods in order to amplify the target polynucleotides.

In a preferred embodiment, the detectable label is a luminescent label. For example, fluorescent labels, bio-luminescent labels, chemi-luminescent labels, and colorimetric labels may be used in the present invention. In a highly preferred embodiment, the label is a fluorescent label, such as a fluorescein, a phosphor, a rhodamine, or a polymethine dye derivative. Examples of commercially available fluorescent labels include, for example, fluorescent phosphoramidites such as FluorePrime (Amersham Pharmacia, Piscataway, N.J.), Fluoredite (Millipore, Bedford, Mass.), FAM (ABI, Foster City, Calif.), and Cy3 or Cy5 (Amersham Pharmacia, Piscataway, N.J.). In another embodiment, the detectable label is a radiolabeled nucleotide.

In a further preferred embodiment, target polynucleotide molecules from a patient sample are labeled differentially from target polynucleotide molecules of a standard. The standard can comprise target polynucleotide molecules from normal individuals (i.e., those not afflicted with cancer). In a highly preferred embodiment, the standard comprises target polynucleotide molecules pooled from samples from normal individuals or tumor samples from individuals having cancer. In another embodiment, the target polynucleotide molecules are derived from the same individual, but are taken at different time points, and thus indicate the efficacy of a treatment by a change in expression of the biomarkers, or lack thereof during and after the course of treatment (i.e., Wnt/β-catenin pathway therapeutic agent), wherein a change in the expression of the biomarkers from a Wnt/β-catenin pathway deregulation pattern to a Wnt/β-eatenin pathway regulation pattern indicates that the treatment is efficacious. In this embodiment, different timepoints are differentially labeled.

### 3.5.2.5 Hybridization to Microarrays

Nucleic acid hybridization and wash conditions are chosen so that the target polynucleotide molecules specifically bind or specifically hybridize to the complementary polynucleotide sequences of the array, preferably to a specific array site, wherein its complementary DNA is located.

Arrays containing double-stranded probe DNA situated thereon are preferably subjected to denaturing conditions to render the DNA single-stranded prior to contacting with the target polynucleotide molecules. Arrays containing single-stranded probe DNA (e.g., synthetic oligodeoxyribonucleic acids) may need to be denatured prior to contacting with the target polynucleotide molecules, e.g., to remove hairpins or dimers which form due to self complementary sequences.

Optimal hybridization conditions will depend on the length (e.g., oligomer versus polynucleotide greater than 200 bases) and type (e.g., RNA, or DNA) of probe and target nucleic acids. One of skill in the art will appreciate that as the oligonucleotides become shorter, it may become necessary to adjust their length to achieve a relatively uniform melting temperature for satisfactory hybridization results. General parameters for specific (i.e., stringent) hybridization conditions for nucleic acids are described in Sambrook et al., MOLECULAR CLONING—A LABORATORY MANUAL (2ND ED.), Vols. 1-3, Cold Spring Harbor Laboratory, Cold Spring Harbor, N.Y. (1989), and in Ausubel et al., CURRENT PROTOCOLS IN MOLECULAR BIOLOGY, vol. 2, Current Protocols Publishing, New York (1994). Typical hybridization conditions for the cDNA microarrays of Schena et al. are hybridization in 5×SSC plus 0.2% SDS at 65° C. for four hours, followed by washes at 25° C. in low stringency wash buffer (1×SSC plus 0.2% SDS), followed by 10 minutes at 25° C. in higher stringency wash buffer (0.1×SSC plus 0.2% SDS) (Schena et al., Proc. Natl. Acad. Sci. U.S.A. 93:10614 (1993)). Useful hybridization conditions are also provided in, e.g., Tijessen, 1993, HYBRIDIZATION WITH NUCLEIC ACID PROBES, Elsevier Science Publishers B. V.; and Kricka, 1992, NONISOTOPIC DNA PROBE TECHNIQUES, Academic Press, San Diego, Calif.

Particularly preferred hybridization conditions include hybridization at a temperature at or near the mean melting temperature of the probes (e.g., within 5° C., more preferably within 2° C.) in 1 M NaCl, 50 mM MES buffer (pH 6.5), 0.5% sodium sarcosine and 30% formamide.

### 3.5.2.6 Signal Detection and Data Analysis

When fluorescently labeled probes are used, the fluorescence emissions at each site of a microarray may be, preferably, detected by scanning confocal laser microscopy. In one embodiment, a separate scan, using the appropriate excitation line, is carried out for each of the two fluorophores used. Alternatively, a laser may be used that allows simultaneous specimen illumination at wavelengths specific to the two fluorophores and emissions from the two fluorophores can be analyzed simultaneously (see Shalon et al., 1996, “A DNA microarray system for analyzing complex DNA samples using two-color fluorescent probe hybridization,” Genome Research 6:639-645, which is incorporated by reference in its entirety for all purposes). In a preferred embodiment, the arrays are scanned with a laser fluorescent scanner with a computer controlled X-Y stage and a microscope objective. Sequential excitation of the two fluorophores is achieved with a multi-line, mixed gas laser and the emitted light is split by wavelength and detected with two photomultiplier tubes. Fluorescence laser scanning devices are described in Schena et al., Genome Res. 6:639-645 (1996), and in other references cited herein. Alternatively, the fiber-optic bundle described by Ferguson et al., Nature Biotech. 14:1681-1684 (1996), may be used to monitor mRNA abundance levels at a large number of sites simultaneously.

Signals are recorded and, in a preferred embodiment, analyzed by computer, e.g., using a 12 or 16 bit analog to digital board. In one embodiment the scanned image is despeckled using a graphics program (e.g., Hijaak Graphics Suite) and then analyzed using an image gridding program that creates a spreadsheet of the average hybridization at each wavelength at each site. If necessary, an experimentally determined correction for “cross talk” (or overlap) between the channels for the two fluors may be made. For any particular hybridization site on the transcript array, a ratio of the emission of the two fluorophores can be calculated. The ratio is independent of the absolute expression level of the cognate gene, but is useful for genes whose expression is significantly modulated in association with the different cancer-related condition.

### 3.6 COMPUTER-FACILITATED ANALYSIS

The present invention further provides for kits comprising the biomarker sets above. In a preferred embodiment, the kit contains a microarray ready for hybridization to target polynucleotide molecules, plus software for the data analyses described above.

The analytic methods described in the previous sections can be implemented by use of the following computer systems and according to the following programs and methods. A Computer system comprises internal components linked to external components. The internal components of a typical computer system include a processor element interconnected with a main memory. For example, the computer system can be an Intel 8086-, 80386-, 80486-, Pentium®, or Pentium®-based processor with preferably 32 MB or more of main memory.

The external components may include mass storage. This mass storage can be one or more hard disks (which are typically packaged together with the processor and memory). Such hard disks are preferably of 1 GB or greater storage capacity. Other external components include a user interface device, which can be a monitor, together with an inputting device, which can be a “mouse”, or other graphic input devices, and/or a keyboard. A printing device can also be attached to the computer.

Typically, a computer system is also linked to network link, which can be part of an Ethernet link to other local computer systems, remote computer systems, or wide area communication networks, such as the Internet. This network link allows the computer system to share data and processing tasks with other computer systems.

Loaded into memory during operation of this system are several software components, which are both standard in the art and special to the instant invention. These software components collectively cause the computer system to function according to the methods of this invention. These software components are typically stored on the mass storage device. A software component comprises the operating system, which is responsible for managing computer system and its network interconnections. This operating system can be, for example, of the Microsoft Windows® family, such as Windows 3.1, Windows 95, Windows 98, Windows 2000, or Windows NT. The software component represents common languages and functions conveniently present on this system to assist programs implementing the methods specific to this invention. Many high or low level computer languages can be used to program the analytic methods of this invention. Instructions can be interpreted during run-time or compiled. Preferred languages include C/C++, FORTRAN and JAVA. Most preferably, the methods of this invention are programmed in mathematical software packages that allow symbolic entry of equations and high-level specification of processing, including some or all of the algorithms to be used, thereby freeing a user of the need to procedurally program individual equations or algorithms. Such packages include Mathlab from Mathworks (Natick, Mass.), Mathematica® from Wolfram Research (Champaign, Ill.), or S-Plus®D from Math Soft (Cambridge, Mass.). Specifically, the software component includes the analytic methods of the invention as programmed in a procedural language or symbolic package.

The software to be included with the kit comprises the data analysis methods of the invention as disclosed herein. In particular, the software may include mathematical routines for biomarker discovery, including the calculation of correlation coefficients between clinical categories (i.e., Wnt/β-catenin signaling pathway regulation status) and biomarker expression. The software may also include mathematical routines for calculating the correlation between sample biomarker expression and control biomarker expression, using array-generated fluorescence data, to determine the clinical classification of a sample.

In an exemplary implementation, to practice the methods of the present invention, a user first loads experimental data into the computer system. These data can be directly entered by the user from a monitor, keyboard, or from other computer systems linked by a network connection, or on removable storage media such as a CD-ROM, floppy disk (not illustrated), tape drive (not illustrated), ZIP® drive (not illustrated) or through the network. Next the user causes execution of expression profile analysis software which performs the methods of the present invention.

In another exemplary implementation, a user first loads experimental data and/or databases into the computer system. This data is loaded into the memory from the storage media or from a remote computer, preferably from a dynamic geneset database system, through the network. Next the user causes execution of software that performs the steps of the present invention.

Alternative computer systems and software for implementing the analytic methods of this invention will be apparent to one of skill in the art and are intended to be comprehended within the accompanying claims. In particular, the accompanying claims are intended to include the alternative program structures for implementing the methods of this invention that will be readily apparent to one of skill in the art.

### EXAMPLES

Examples are provided below to further illustrate different features and advantages of the present invention. The examples also illustrate useful methodology for practicing the invention. These examples do not limit the claimed invention.

### Materials and Methods:

Reagents

DLD1, SW620, and SW480 cells were obtained from ATCC and cultured in DMEM supplemented with 10% fetal bovine serum and 5% penicillin/streptomycin. All siRNA transfections, irrespective of cell type, employed RNAiMAX (Invitrogen, Carlsbad, Calif.).

High Throughput siRNA Screens

The genome-wide siRNA screens were performed as previously described, with minor modifications (Bartz et al., 2006, Mol. Cell. Biol. 26:9377-9386). Briefly, cells were reverse-transfected in 1536-well plates, with a final concentration of pooled siRNA at 25 nM. 72 hours post-transfection, firefly luciferase and Renilla luciferase were quantitated. The pilot-scale screens were completed essentially as described above, except that cells were reverse-transfected in 384-well plates and cell viability was controlled by alamarBlue staining.

Gene Expression Studies

For determination of the Wnt/β-catenin signature, the cell lines DLD1, SW480, and SW620 (derived from colorectal tumor or metastases) were transfected with the indicated siRNAs, or water (mock), with RNAiMAX (Invitrogen, Carlsbad, Calif.). The cell lines were plated at 1.5E5 cells per well in tissue culture coated 6-well plates in 2 ml of media. The cells were cultured for 24 hours at 37° C. in 5% CO₂. Lipofectamine RNAiMAX was added to OptiMEM media (Gibco) to a final concentration of 31 μl/ml. The mixture was incubated at room temperature for approximately 5 minutes. Next, 475 μl of the OptiMEM/Lipfectamine RNAiMAX mixture was combined with 25 μl of 10 μM siRNA in water and gently mixed. For “mock” transfections, 25 μl of water was substituted for the siRNA. The siRNA and transfection reagent medium were incubated at room temperature for 20 minutes. Next, 400 μl of the mixture was added to the cell line previously plated in 2 ml.

Following the 72-hour incubation, RNA was extracted from the cells using RNEasy kits (Qiagen, Valencia, Calif.) following manufacturer's protocols, including the on-column DNAse digestion step. Samples were frozen at −80° C. and then submitted for microarray gene expression profiling on the Affymetrix platform.

For the microfluidic QPCR tertiary screen data, pools of three siRNAs to each “hit” target were transfected into DLD1 cells. RNA was isolated after 72 hours using the RNEasy Mini 96-well plate kit (Qiagen, Valencia, Calif.). This RNA was run in a RT reaction using the ABI Archive Kit (Applied Biosystems, Foster City, Calif.). The resulting cDNA was run in a pre-amp reaction using the ABI Pre-Amp Master Mix (Applied Biosystems, Foster City, Calif.). On-Demand TagMan Assays (Applied Biosystems, Foster City, Calif.) for the Wnt/β-catenin pathway signature transcripts to be assayed were mixed with Biomark Assay Loading Buffer (Fluidigm, San Francisco, Calif.) in preparation for loading. The BioMark 46.46 chip creates all possible combinations of 46 assay wells and 46 sample wells for a total of 2116 QPCR reactions. In this experiment, duplicate assays were loaded so that duplicate C_{T} values for each sample could be obtained. The chip was run in the Biomark System (Fluidigm, San Francisco, Calif.) instrument for 40 cycles. The entire process from transfections to QPCR was run three times.

Tertiary Screen Data Analysis

C_{T} values from the three Biomark runs were converted to fold changes using standard calculations, using GUSB (NM_{—}000181, SEQ ID NO:379) as the input control and duplicate mock transfected samples as the negative control. Additionally, GAPDH (NM_{—}002046, SEQ ID NO: 380) and HPRT1 (NM_{—}000194, SEQ ID NO: 381) were run as input controls. Since no siRNA pools tested target these genes, the regulation of these genes by all siRNAs can be used as the negative control sample in a t-test. The six replicate regulations of a given siRNA pool on a given pathway signature gene are used as the positive control sample. In this way, p-values were calculated for each siRNA pool/signature gene combination. The Bonferroni correction was applied. Only gene regulations whose p-value was less than 0.05 were considered significant.

For a given siRNA pool, the overlap with an Wnt/β-catenin pathway 18-gene signature subset tested in the Biomark qPCR platform was evaluated by counting the number of genes that were both significantly regulated and were regulated in the proper direction. For siRNA pools that negatively regulate BAR, the proper direction was regulating the gene in the same direction as CTNNB1. For a gene that positively regulates BAR, the proper direction was regulating the gene in the opposite direction as CTNNB1. A p-value was calculated from this count using the binomial distribution assuming the odds of a gene being regulated at random by a given siRNA to be 1 out of 20. These p-values were Bonferroni corrected. Only those siRNA pools with a p-value less than 0.01 were considered to significantly overlap with the tested 18-gene Wnt/β-catenin pathway signature set.

For cDNA microarray expression analyses, p-values were placed on the regulation of a gene using a prioprietary error-model. A p-value less than 0.01 was considered significant. The p-value of a siRNA's signature overlap with the Wnt/β-catenin pathway signature was calculated using the hypergeometric distribution and Bonferroni corrected. A p-value less than 0.01 was considered a significant signature overlap.

### Example 1

### Genome Scale siRNA Screen to Identify Potential Regulators of the Wnt/β-Catenin Pathway

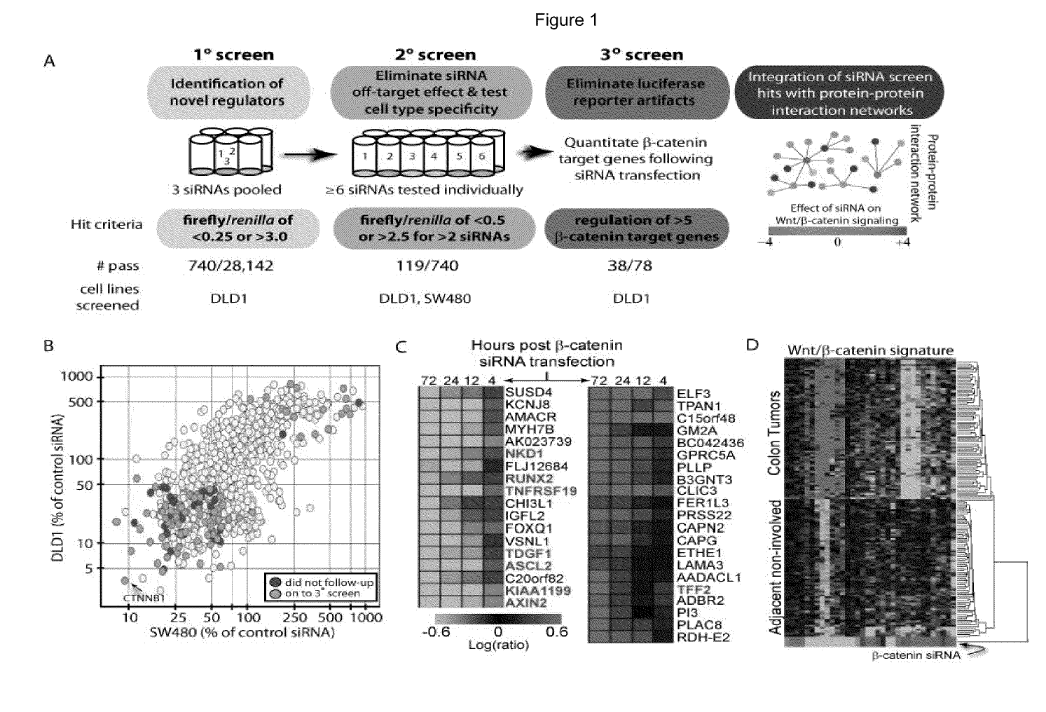

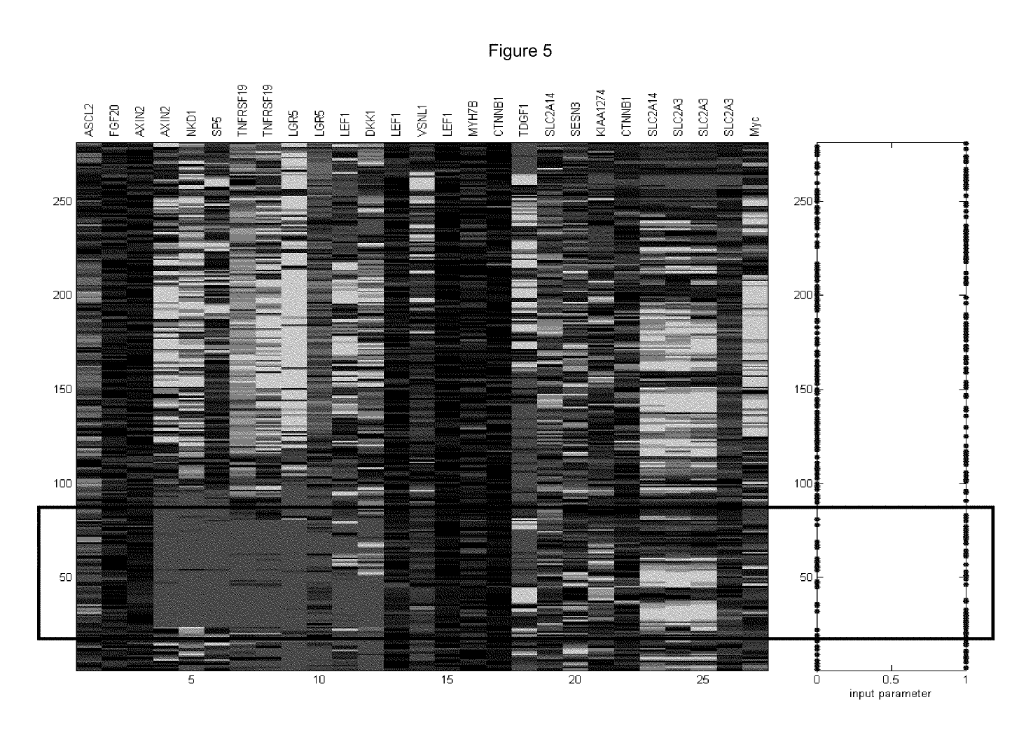

A genome-wide small interfering RNA (siRNA) screen on the Wnt/β-catenin pathway was performed in human DLD1 colon adenocarcinoma cells (FIG. 1A). These cells harbor inactivating mutations in APC, and consequently display constitutively active Wnt/β-catenin signal transduction (Morin et al., 1997, Science 275:1787-1790). To enable high-throughput quantitation of Wnt/β-catenin signal transduction, DLD1 cells were engineered to co-express a β-catenin-responsive firefly luciferase reporter (BAR) and an EF1α-driven Renilla luciferase reporter for normalization purposes (Major et al., 2007, Science 316:1043-1046). Using these cells, 28,124 siRNA pools targeting 20,042 mRNAs were screened in triplicate (FIG. 5). siRNAs targeting 3% of these mRNAs fulfilled the criteria of a normalized fold change of greater than 3 or less than 0.25 with a t-test p-value less than 0.01. Therefore, with stringent data restriction, this primary screen yielded 740 genes that regulate Wnt/β-catenin pathway signal transduction.

### Example 2

### Secondary Validation Screens

As the off-target silencing effects inherent to siRNA screens can produce high false-positive discovery rates (Echeverri et al., 2006, Nat. Methods 3:777-779; Jackson et al., 2006, RNA 12:1179-1187), three validation screens were implemented, the first to increase the number of siRNAs tested, the second to eliminate cell-type specific hits, and a rigorous third screen to insure that the hits were indeed regulating endogenous β-catenin target genes. In the first of these validation screens, we individually tested at least three, and on average six, non-overlapping gene-specific siRNAs (FIG. 1B). Of the 740 genes that passed our hit criteria in the primary screen, 268 were confirmed by a minimum of 2-independent siRNAs (data not shown).

In the second validation screen, the general applicability of our discoveries was broadened by eliminating cell line-specific siRNA hits. Specifically, the secondary screen was repeated by individually testing three to six independent siRNAs in SW480 cells, an APC mutant colorectal adenocarcinoma cell line (Goyette et al., 1992, Mal. Cell. Biol. 12:1387-1395). 119 genes were identified at the intersection of secondary screen datasets for DLD1 cells and SW480 cells (FIG. 1B). Therefore, of the 28,124 siRNAs tested against the Wnt/β-catenin pathway, 119 genes were identified by the secondary screens that by definition validate with multiple siRNAs in multiple cell lines.

### Example 3

### Definition of the Wnt/β-Catenin Pathway Signature

For the final validation screen, endogenous β-catenin regulated genes were used to monitor Wnt/β-catenin pathway activity (FIG. 1A). The rationale for this added layer of validation is that the primary and secondary screens employed an artificial β-catenin reporter system. Thus, to test whether the candidate mRNAs were legitimate modulators of endogenous β-catenin target genes, a Wnt/β-catenin pathway signature in colon cancer cells had to be defined.

Using genome-wide cDNA microarray expression analyses, five non-overlapping β-catenin-specific siRNAs in DLD1 cells were profiled (see Table 2). All of the siRNAs targeting β-catenin (CTNNB1, NM_{—}001098209, SEQ ID NO: 83) were shown to down-regulate transcript levels to below 10% remaining. For DLD1, a Wnt/β-catenin pathway signature was created by finding those transcripts which are regulated at least two-fold (either direction) with a p-value less than 0.01 by all five siRNAs used and which are not significantly regulated by the luciferase negative control siRNA. Of the 43,675 transcripts measured, 329 were regulated by all five β-catenin siRNAs (FIG. 6). SW480 and SW620 cells treated with the two β-catenin-specific siRNAs were profiled and compared with the DLD1 cells (see Table 2). These samples were hybridized against luciferase control siRNA rather than the “mock” transfection sample, so the Wnt/β-catenin pathway signature in these two cell lines were created by identifying those transcripts regulated at least two-fold (either direction) with a p-value less than 0.01 by both of the two CTNNB1-targeting siRNAs. 38 genes were identified in common among the sets of experiments that define the colonic Wnt/β-catenin pathway gene signature (FIG. 1C; Table 3).

TABLE 2
siRNAs and cell lines used for identifying
Wnt/β-catenin pathway signature

| | | SEQ | | | |
|---|---|---|---|---|---|
| Target | | ID | | | |
| Gene | Guide Sequence | NO: | DLD | SW480 | SW620 |
| Luci- | UCGAAGUAUUCCGCGUACGTT | 382 | Yes | Yes | Yes |
| ferase | | | | | |
| CTNNB1 | CUAUCUGUCUGCUCUAGUATT | 383 | Yes | Yes | Yes |
| CTNNB1 | GAGGACCUAUACUUACGAATT | 384 | Yes | Yes | Yes |
| CTNNB1 | CUCAGAUGGUGUCUGCUAUTT | 385 | Yes | No | No |
| CTNNB1 | CCACUAAUGUCCAGCGUUUTT | 386 | Yes | No | No |
| CTNNB1 | GAAUGAAGGUGUGGCGACATT | 387 | Yes | No | No |

TABLE 3
Wnt/β-catenin pathway gene signature and microarray
probe sequences.

| | | | Transcript | | Probe |
|---|---|---|---|---|---|
| | Representative | | SEQ ID | | SEQ ID |
| Gene Symbol | Transcript | Arm | NO: | Probe Sequence | NO: |
| None | AK023739 | Up | 388 | ATGGTCATACGATTCTTATGGACCC | 426 |
| None | BC042436 | Down | 389 | AGAAATCCTCTAGTTGGTGCCTCCA | 427 |
| RDHE2 | NM_138969 | Down | 390 | TGAGGCATTTTCCTGCAGAATGGGC | 428 |
| AADACL1 | NM_020792 | Down | 391 | CAGGGTTCCTTCAATTGGCATTTTC | 429 |
| ADRB2 | NM_000024 | Down | 392 | AAGGAAGGGCATCCTTCTGCCTTTT | 430 |
| AMACR | NM_014324 | Up | 393 | GATGTATGCACCTATTGGACACTGT | 431 |
| ASCL2 | NM_005170 | Up | 394 | AAACGGGCTTGGAGCTGGCCCCATA | 432 |
| AXIN2 | NM_004655 | Up | 395 | GGTTCTGGCTATGTCTTTGCACCAG | 433 |
| B3GNT3 | NM_014256 | Down | 396 | TGTCTGCCAGTCAAGCTTCACAGGC | 434 |
| C15orf48 | NM_032413 | Down | 397 | GAGCCTCATCTTTCGCTGTGTATTC | 435 |
| C20orf82 | NM_080826 | Up | 398 | TTATTTGGTCATGAGGCACCTTGCT | 436 |
| CAPG | NM_001747 | Down | 399 | AGGCCGCAGCTCTGTATAAGGTCTC | 437 |
| CAPN2 | NM_001748 | Down | 400 | CACCTCTGTCGCTTGGGTTAAACAA | 438 |
| CHI3L1 | NM_001276 | Up | 401 | GTAAGACTCGGGATTAGTACACACT | 439 |
| CLIC3 | NM_004669 | Down | 402 | CTGCTCTATGACAGCGACGCCAAGA | 440 |
| ELF3 | NM_004433 | Down | 403 | GAGGTCGTGCGCAGGTTTGTTACAT | 441 |
| ETHE1 | NM_014297 | Down | 404 | ACCTTGTACCACTCGGTCCATGAAA | 442 |
| FER1L3 | NM_013451 | Down | 405 | ATCAGGTCGTCAATCTTCAGATCAA | 443 |
| FOXQ1 | NM_033260 | Up | 406 | TTTAGTTTCTTTGCGAAGCCTGCTC | 444 |
| GM2A | NM_000405 | Down | 407 | GCTGTGCAGTGTGATGTGTCCCAAA | 445 |
| GPRC5A | NM_003979 | Down | 408 | TGCTCCTCTAACTCACAGTGGGTTT | 446 |
| IGFL2 | NM_001002915 | Up | 409 | CAGCGCCAGGAAATCATGAGGTTCA | 447 |
| KCNJ8 | NM_004982 | Up | 410 | GATTGACAAGCGCAGTCCCCTGTAT | 448 |
| KIAA1199 | NM_018689 | Up | 411 | ATGTCCTTCTTGTCCACGGTTTTGT | 449 |
| LAMA3 | NM_198129 | Down | 412 | AGGTGCTCCAGCCAATTTGACGACA | 450 |
| MYH7B | NM_020884 | Up | 413 | AGCATGAGCGCCGTGTCAAGGAGCT | 451 |
| NKD1 | NM_033119 | Up | 414 | AGAGACCCTCGAAATCTCCGAGAAG | 452 |
| PI3 | NM_002638 | Down | 415 | GATTGGTATGGCCTTAGCTCTTAGC | 453 |
| PLAC8 | NM_016619 | Down | 416 | GTGACTGTTTCAGCGACTGCGGAGT | 454 |
| PLLP | NM_015993 | Down | 417 | GTCTCTGCCTTGTCTTTAGAGGACT | 455 |
| PRSS22 | NM_022119 | Down | 418 | GAGCGGGATGCTTGTCTGGGCGACT | 456 |
| RUNX2 | NM_001015051 | Up | 419 | TTTAAATGGTTAATCTCCGCAGGTC | 457 |
| SUSD4 | NM_017982 | Up | 420 | AACAAAGCTCTGATCCTTAAAATTG | 458 |
| TDGF1 | NM_003212 | Up | 421 | GTGGACCTTAGAATACAGTTTTGAG | 459 |
| TFF2 | NM_005423 | Down | 422 | ATAACAGGACGAACTGCGGCTTCCC | 460 |
| TNFRSF19 | NM_148957 | Up | 423 | CATTTTACCCTACCTACTCTAGAAA | 461 |
| TSPAN1 | NM_005727 | Down | 424 | GAGGGTTGCTTCAATCAGCTTTTGT | 462 |
| VSNL1 | NM_003385 | Up | 425 | GACACAGAGGGACCCTTGGCTCCTG | 463 |

TABLE 4a
Wnt/β-catenin pathway gene signature - up arm

| Gene Symbol | Representative Transcript | Arm | SEQ ID NO: |
|---|---|---|---|
| None | AK023739 | Up | 388 |
| AMACR | NM_014324 | Up | 393 |
| ASCL2 | NM_005170 | Up | 394 |
| AXIN2 | NM_004655 | Up | 395 |
| C20orf82 | NM_080826 | Up | 398 |
| CHI3L1 | NM_001276 | Up | 401 |
| FOXQ1 | NM_033260 | Up | 406 |
| IGFL2 | NM_001002915 | Up | 409 |
| KCNJ8 | NM_004982 | Up | 410 |
| KIAA1199 | NM_018689 | Up | 411 |
| MYH7B | NM_020884 | Up | 413 |
| NKD1 | NM_033119 | Up | 414 |
| RUNX2 | NM_001015051 | Up | 419 |
| SUSD4 | NM_017982 | Up | 420 |
| TDGF1 | NM_003212 | Up | 421 |
| TNFRSF19 | NM_148957 | Up | 423 |
| VSNL1 | NM_003385 | Up | 425 |

TABLE 4b
Wnt/β-catenin pathway gene signature - down arm

| Gene Symbol | Representative Transcript | Arm | SEQ ID NO: |
|---|---|---|---|
| None | BC042436 | Down | 389 |
| RDHE2 | NM_138969 | Down | 390 |
| AADACL1 | NM_020792 | Down | 391 |
| ADRB2 | NM_000024 | Down | 392 |
| B3GNT3 | NM_014256 | Down | 396 |
| C15orf48 | NM_032413 | Down | 397 |
| CAPG | NM_001747 | Down | 399 |
| CAPN2 | NM_001748 | Down | 400 |
| CLIC3 | NM_004669 | Down | 402 |
| ELF3 | NM_004433 | Down | 403 |
| ETHE1 | NM_014297 | Down | 404 |
| FER1L3 | NM_013451 | Down | 405 |
| GM2A | NM_000405 | Down | 407 |
| GPRC5A | NM_003979 | Down | 408 |
| LAMA3 | NM_198129 | Down | 412 |
| PI3 | NM_002638 | Down | 415 |
| PLAC8 | NM_016619 | Down | 416 |
| PLLP | NM_015993 | Down | 417 |
| PRSS22 | NM_022119 | Down | 418 |
| TFF2 | NM_005423 | Down | 422 |
| TSPAN1 | NM_005727 | Down | 424 |

To validate this signature set, a time course analysis was conducted. 31 of the 38 signature genes were regulated within 24 hours of β-catenin silencing, suggesting that the majority of the transcripts comprising this gene signature were directly regulated by β-catenin (FIG. 1C). As further validation, we hypothesized that a Wnt/β-catenin gene signature would discriminate normal colon from colon cancer, given that ˜90% of colon cancers have activated Wnt/β-catenin signaling (Segditsas and Tomlinson, 2006, 25:7531-7537; Polakis, 2000, Genes Dev. 14:1837-1851). Unsupervised hierarchical clustering of 69 matched colon tumors and adjacent uninvolved tissues using this gene signature correctly assigned all of the tumors and 66 of the 69 matched normal samples (FIG. 1D). Samples were classified by comparing the expression of biomarkers in the tumor samples to their expression in CTNNB1 knockdown/DLD-1 cells. Since WNT is constitutively activated in DLD-1 cells, and it is suggested to be also constitutively active in as many as 90% of colon tumors, the expression of biomarkers in tumors when compared to normal adjacent tissue should be opposite of their expression in DLD cells following CTNNB1 knockdown. For example, AXIN2 transcript levels are mostly higher in colon tumor samples compared to normal adjacent tissue samples, and AXIN2 transcript levels decrease in DLD-1 cells following CTNNB1 knockdown. Comparative examination of the DLD1 Wnt/β-catenin pathway profile revealed up-regulation of β-catenin target genes in cancer as compared to normal.

### Example 4

### Tertiary Validation Screen

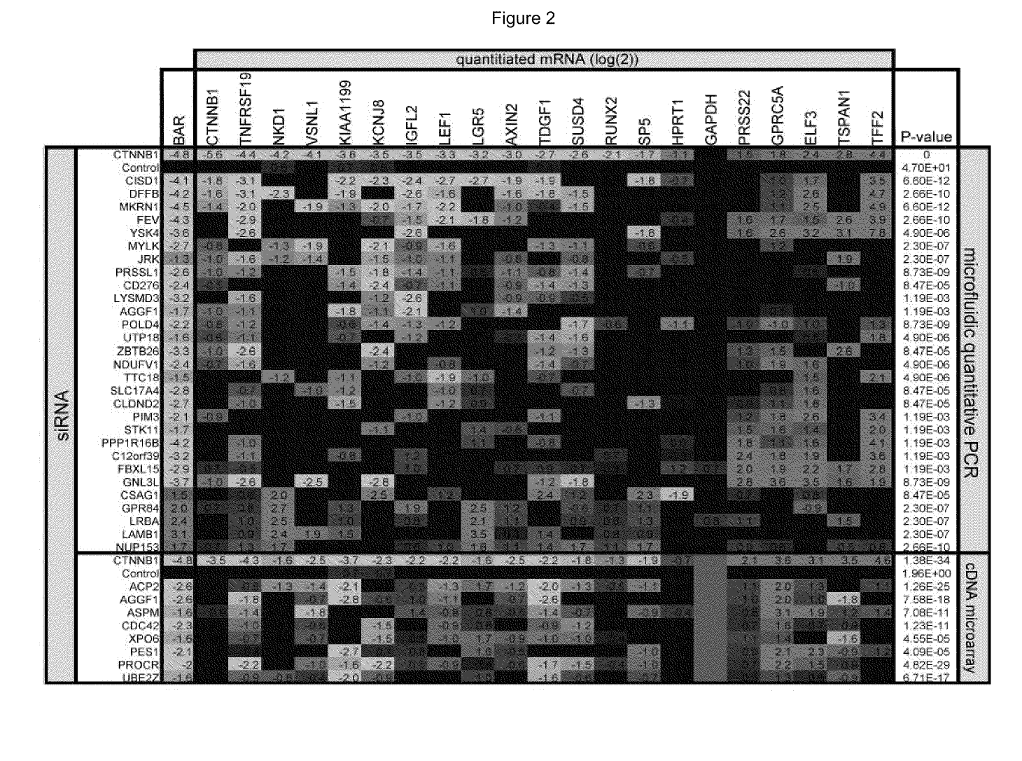

Using a subset of the Wnt/β-catenin pathway signature as an endogenous readout for signal transduction, we next asked how genes identified in the secondary screen regulate Wnt/β-catenin pathway signaling. A microfluidic real time PCR platform was employed to simultaneously quantitate the expression of 18 β-catenin target genes in 77 different samples, each of which represents a siRNA secondary screen hit (FIG. 2). 29 of the 77 siRNAs tested regulated at least 6 endogenous Wnt/β-catenin target genes (p-value<0.01). Additionally, cDNA genome-wide microarray expression analysis for 10 of the 13 screen hits examined confirmed against an 18-gene subset of the Wnt/β-catenin pathway gene signature (FIG. 2). When this 18-gene subset of the Wnt/β-catenin pathway signature was applied was applied as a third filter for hits passing the first two validation screens, our siRNA screens reduced 740 hits from the primary screen of over 20,000 mRNAs to 38 triply validated regulators of Wnt/β-catenin pathway signal transduction (Table 5).

TABLE 5
38 Triply validated regulators of Wnt/β-catenin pathway

| | | SEQ | | Tertiary Screen | DLD-1-BAR |
|---|---|---|---|---|---|
| | | ID | | Corr Overlap P- | Regulation |
| Gene Symbol | Transcript | NO: | Screen | value | (LOG2(Fold)) |
| CISD1 | NM_018464 | 464 | Genome | 6.60E−12 | −4.13 |
| MKRN1 | NM_013446 | 465 | Genome | 6.60E−12 | −4.50 |
| DFFB | NM_004402 | 466 | Genome | 2.66E−10 | −4.17 |
| FEV | NM_017521 | 467 | Genome | 2.66E−10 | −4.32 |
| NUP153 | NM_005124 | 468 | Genome | 2.66E−10 | 1.73 |
| PRSSL1 | NM_214710 | 469 | Genome | 8.73E−09 | −2.55 |
| POLD4 | NM_021173 | 470 | Genome | 8.73E−09 | −2.23 |
| GNL3L | NM_019067 | 471 | Genome | 8.73E−09 | −3.65 |
| MYLK | NM_053025 | 472 | Genome | 2.30E−07 | −2.68 |
| JRK | NM_003724 | 473 | Genome | 2.30E−07 | −1.27 |
| GPR84 | NM_020370 | 474 | Genome | 2.30E−07 | 2.04 |
| LRBA | NM_006726 | 475 | Genome | 2.30E−07 | 2.42 |
| LAMB1 | NM_002291 | 476 | Genome | 2.30E−07 | 3.07 |
| YSK4 | NM_001018046 | 477 | Genome | 4.90E−06 | −3.63 |
| UTP18 | NM_016001 | 478 | Genome | 4.90E−06 | −1.62 |
| NDUFV1 | NM_007103 | 479 | Genome | 4.90E−06 | −2.40 |
| TTC18 | NM_145170 | 480 | Genome | 4.90E−06 | −1.51 |
| CD276 | NM_001024736 | 481 | Genome | 8.47E−05 | −2.42 |
| ZBTB26 | NM_020924 | 482 | Genome | 8.47E−05 | −3.26 |
| SLC17A4 | NM_005495 | 483 | Genome | 8.47E−05 | −2.76 |
| CLDND2 | NM_152353 | 484 | Genome | 8.47E−05 | −2.70 |
| CSAG1 | NM_001102576 | 485 | Genome | 8.47E−05 | 1.51 |
| LYSMD3 | NM_198273 | 486 | Genome | 1.19E−03 | −3.20 |
| AGGF1 | NM_018046 | 487 | Genome | 1.19E−03 | −1.67 |
| PIM3 | NM_001001852 | 488 | Genome | 1.19E−03 | −2.13 |
| STK11 | NM_000455 | 489 | Genome | 1.19E−03 | −1.73 |
| PPP1R16B | NM_015568 | 490 | Genome | 1.19E−03 | −4.21 |
| C12orf39 | NM_030572 | 491 | Genome | 1.19E−03 | −3.24 |
| FBXL15 | NM_024326 | 492 | Genome | 1.19E−03 | −2.93 |
| AGGF1 | NM_018046 | 487 | Pilot | 2.33E−15 | −2.63 |
| SLC25A39 | NM_016016 | 493 | Pilot | 3.02E−14 | −0.43 |
| AKT1 | NM_001014431 | 494 | Pilot | 4.55E−12 | −1.25 |
| UBE2Z | NM_023079 | 495 | Pilot | 4.98E−12 | −1.57 |
| PROCR | NM_006404 | 496 | Pilot | 1.34E−06 | −2.01 |
| ASPM | NM_018136 | 497 | Pilot | 3.84E−04 | −1.60 |
| PES1 | NM_014303 | 498 | Pilot | 1.76E−03 | −2.09 |
| XPO6 | NM_015171 | 499 | Pilot | 1.10E−02 | −1.56 |
| ACP2 | NM_001610 | 500 | Pilot | 2.16E−02 | −2.64 |
| CDC42 | NM_001039802 | 501 | Pilot | 4.50E−02 | −2.25 |

### Example 5

### Wnt/β-Catenin Signaling Pathway is Deregulated in a Subpopulation of HCC Patients

The Wnt/β-catenin pathway biomarkers derived from the colon cancer cells lines were assayed in hepatocellular carcinoma (HCC) tumor samples. More than 200 matched HCC tumors and the corresponding adjacent non-tumor tissues were profiled on the Affymetrix oligo arrays. The expression profiling data from each individual sample was ratioed to the mean of all the expression profiles including both the tumor and adjacent non-tumor tissues. Wnt/β-catenin pathway biomarkers derived from the colon cell lines were analyzed in HCC tumors, by 2-dimesional clustering based on the gene expression levels. Multiple genes from the reporter list showed coherent co-regulation patterns. Approximately 30% of the HCC patents have deregulation Wnt/β-catenin pathway signaling (see FIG. 5) and patients with pathway deregulation seem to be associated with high recurrence (boxed in FIG. 5, where there are fewer patients in the “no-recurrence” category).

What is claimed is:

1. A method for classifying an isolated cell sample as having a deregulated or regulated Wnt/β-catenin signaling pathway, comprising
(i) calculating a signature score by a method comprising: a) calculating a differential expression value of a first expression level of each of a first plurality of genes and each of a second plurality of genes in the cell sample relative to a second expression level of each of said first plurality of genes and each of said second plurality of genes in a control cell sample, said first plurality of genes consisting of at least 3 or more of the genes for which biomarkers are listed in Table 4a and said second plurality of genes consisting of at least 3 or more of the genes for which biomarkers are listed in Table 4b; b) calculating the mean differential expression values of the expression levels of said first plurality of genes and said second plurality of genes; and c) subtracting said mean differential expression value of said second plurality of genes from said mean differential expression value of said first plurality of genes to obtain said signature score;
(ii) classifying said cell sample as having a deregulated Wnt/β-catenin signaling pathway a) if said obtained signature score is above a predetermined threshold, and b) if said signature score is statistically significant; and
(iii) displaying; or outputting to a user interface device, a computer readable storage medium, or a local or remote computer system; the classification produced by said classifying step (ii).

2. The method of claim 1, wherein said differential expression value is log(10) ratio.

3. The method of claim 1, wherein said threshold is 0.

4. The method of claim 1, wherein said signature scores is statistically significant if it has a p-value less than 0.05.

5. The method of claim 1, wherein the isolated cell sample is from a human subject.

6. The method of claim 1, wherein said first plurality consists of at least 5 of the genes for which biomarkers are listed in Table 4a and said second plurality consists of at least 5 of the genes for which biomarkers are listed in Table 4b.

7. A method for predicting response of a subject to an agent that modulates the Wnt/β-catenin signaling pathway, said method comprising:
(a) classifying said subject as having a deregulated or regulated Wnt/β-catenin signaling pathway, wherein said classifying comprises:
(i) calculating a signature score by a method comprising: a) calculating a differential expression value of a first expression level of each of a first plurality of genes and each of a second plurality of genes in an isolated cell sample derived from said subject relative to a second expression level of each of said first plurality of genes and each of said second plurality of genes in a control cell sample, said first plurality of genes consisting of at least 3 or more of the genes for which biomarkers are listed in Table 4a and said second plurality of genes consisting of at least 3 or more of the genes for which biomarkers are listed in Table 4b; b) calculating the mean differential expression values of the expression levels of said first plurality of genes and said second plurality of genes; and c) subtracting said mean differential expression value of said second plurality of genes from said mean differential expression value of said first plurality of genes to obtain said signature score;
(ii) classifying said subject as having a deregulated Wntβ-catenin signaling pathway a) if said obtained signature score is above a predetermined threshold, and b) if said signature score is statistically significant; and
(iii) displaying; or outputting to a user interface device, a computer readable storage medium, or a local or remote computer system; the classification produced by said classifying step (ii);
wherein a subject classified as having deregulated Wnt/β-catenin signaling pathway is indicative of a subject that is predicted to respond to the agent.

8. The method of claim 7, wherein said differential expression value is log(10) ratio.

9. The method of claim 7, wherein said threshold is 0.

10. The method of claim 7, wherein said signature scores is statistically significant if it has a p-value less than 0.05.

11. The method of claim 7, wherein the isolated cell sample is from a human subject.

12. The method of claim 7, wherein said first plurality consists of at least 5 of the genes for which biomarkers are listed in Table 4a and said second plurality consists of at least 5 of the genes for which biomarkers are listed in Table 4b.

13. A method of determining whether an agent modulates the Wnt/β-catenin signaling pathway in a subject, comprising:
(a) contacting a subject with an agent;
(b) classifying said subject as having a deregulated or regulated Wnt/β-catenin signaling pathway, wherein said classifying comprises:
(i) calculating a signature score by a method comprising: a) calculating a differential expression value of a first expression level of each of a first plurality of genes and each of a second plurality of genes in an isolated cell sample derived from said subject relative to a second expression level of each of said first plurality of genes and each of said second plurality of genes in a control cell sample not contacted with said agent, said first plurality of genes consisting of at least 3 or more of the genes for which biomarkers are listed in Table 4a and said second plurality of genes consisting of at least 3 or more of the genes for which biomarkers are listed in Table 4b; b) calculating the mean differential expression values of the expression levels of said first plurality of genes and said second plurality of genes; and c) subtracting said mean differential expression value of said second plurality of genes from said mean differential expression value of said first plurality of genes to obtain said signature score;
(ii) classifying said subject as having a regulated Wnt/β-catenin signaling pathway a) if said obtained signature score is below a predetermined threshold, and b) if said signature score is statistically significant; and
(iii) displaying; or outputting to a user interface device, a computer readable storage medium, or a local or remote computer system; the classification produced by said classifying step (ii);
wherein a subject treated with said agent and classified as having regulated Wnt/β-catenin signaling pathway is indicative of an agent that modulates the Wnt/β-catenin signaling pathway.

14. The method of claim 13, wherein said differential expression value is log(10) ratio.

15. The method of claim 13, wherein said threshold is 0.

16. The method of claim 13, wherein said signature scores is statistically significant if it has a p-value less than 0.05.

17. The method of claim 13, wherein the isolated cell sample is from a human subject.

18. The method of claim 13, wherein said first plurality consists of at least 5 of the genes for which biomarkers are listed in Table 4a and said second plurality consists of at least 5 of the genes for which biomarkers are listed in Table 4b.
